(19) United States
(12) Patent Application Publication
(10) Pub. No.: US 2024/0239793 A1
(43) Pub. Date: Jul. 18, 2024

(54) PROCESS FOR PREPARING PEXIDARTINIB

(71) Applicant: JOHNSON MATTHEY PUBLIC LIMITED COMPANY, London (GB)

(72) Inventors: Johannes SCHRANCK, Paulsboro, NJ (US); Eric SLACK, Paulsboro, NJ (US)

(21) Appl. No.: 18/557,217

(22) PCT Filed: Jun. 13, 2022
(86) PCT No.: PCT/GB2022/051484

§ 371 Date: Oct. 25, 2023

Related U.S. Application Data
(60) Provisional application No. 63/210,718, filed on Jun. 15, 2021.

(51) Int. Cl. C07D 471/04; A61K 31/444

(52) U.S. Cl. CPC C07D 471/04; A61K 31/444

(57) ABSTRACT

The present invention relates to a process for the preparation of a compound of formula (VI), or a salt or derivative thereof, comprising reacting a compound of formula (V) with a compound of formula (IX) wherein R², R⁴, R⁵ and Z are as described in the specification; in the presence of a Group 10 transition metal catalyst, a base, and water to give the compound of formula (VI).

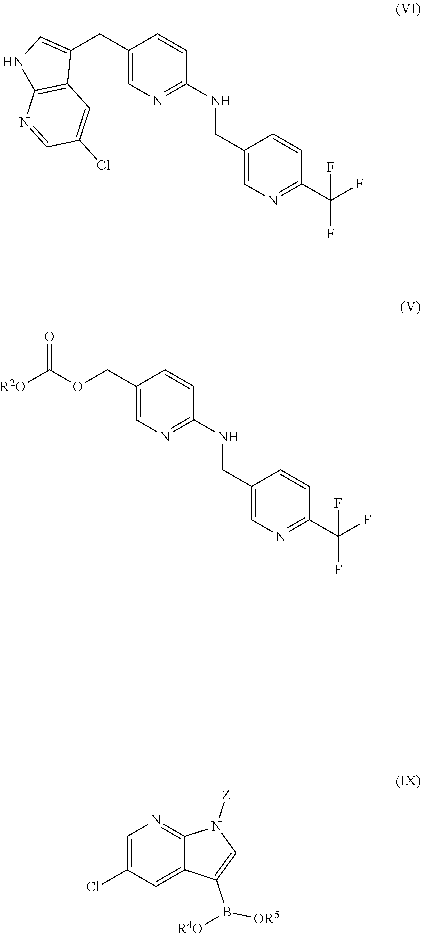

### FIELD OF THE INVENTION

The present invention relates to a new process for preparing pexidartinib, or a salt or derivative thereof, as well as to intermediates made during its synthesis.

### BACKGROUND TO THE INVENTION

Pexidartinib (shown below) is a kinase inhibitor drug used to treat tenosynovial giant cell tumor (TGCT).

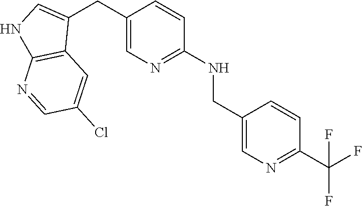 [SMILES: FC(F)(F)c1ccc(CNc2ccc(Cc3c[nH]c4ncc(Cl)cc34)cn2)cn1]

Synthetic routes to pexidartinib have been described, for example in WO2016/179412, WO2008/063888, and CN110156775. WO2016/179412 describes a multi-step process for the preparation of pexidartinib. The process involves the preparation of a 2-[(di-tert-butoxycarbonyl)amino]-5-formylpyridine intermediate, the steps to which are poor-yielding and/or require the use of toxic and/or expensive reagents. Later steps in the synthesis also involve the use of toxic and corrosive reagents, such as trifluoroacetic acid and triethyl silane, in superstoichiometic quantities. Additionally, triethyl silane is highly pyrophoric. Similar drawbacks occur in the processes described in WO2008/063888 and CN110156775.

Thus, there exists a need to provide new processes for the preparation of pexidartinib, which avoid the need for expensive and/or toxic and corrosive reagents and allow the use of milder conditions. In line with this, there is also a desire to achieve a more environmentally friendly route to this important pharmaceutical compound.

### SUMMARY OF THE INVENTION

Viewed from a first aspect, the present invention provides a process for the preparation of a compound of formula (VI), or a salt or derivative thereof,

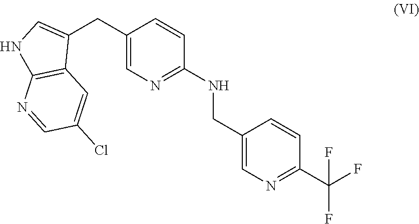 [SMILES: FC(F)(F)c1ccc(CNc2ccc(Cc3c[nH]c4ncc(Cl)cc34)cn2)cn1]

comprising reacting a compound of formula (V)

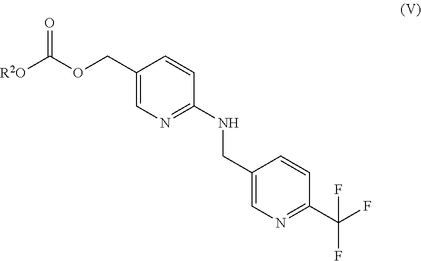

wherein R² is a substituted or unsubstituted C₁₋₂₀ straight-chain or C₃₋₂₀ branched-chain alkyl group,
with a compound of formula (IX)

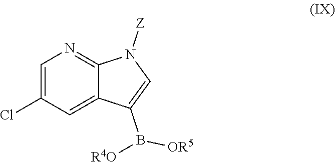

wherein
Z is —SO₂R″ or —CO₂R³;
R″ is a substituted or unsubstituted C₁₋₂₀ straight-chain or C₃₋₂₀ branched-chain alkyl group or a substituted or unsubstituted C₆₋₂₀ aryl group;
R³ is a substituted or unsubstituted C₁₋₂₀ straight-chain or C₃₋₂₀ branched-chain alkyl group or a substituted or unsubstituted C₆₋₂₀ aryl group; and
R⁴ and R⁵ are, independently, H or a substituted or unsubstituted organic group comprising 1-20 carbon atoms; or, together with the atoms to which they are attached, R⁴ and R⁵ form a ring;
in the presence of a Group 10 transition metal catalyst, a base, and water to give said compound of formula (VI).

In a preferred process of the present invention, the compound of formula (V) is prepared in situ in the presence of said compound of formula (IX), said Group 10 transition metal catalyst, said base and said water by reacting a compound of formula (IV), or a salt thereof,

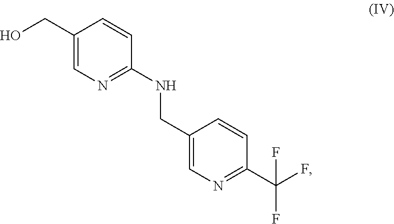 [SMILES: OCc1ccc(NCc2ccc(C(F)(F)F)nc2)nc1]

with a dialkyl carbonate of formula R²O(CO)OR², wherein each R² is a substituted or unsubstituted C₁₋₂₀ straight-chain or C₃₋₂₀ branched-chain alkyl group.

In an alternative preferred process of the present invention, the compound of formula (V) is prepared by reacting a compound of formula (IV), or a salt thereof,

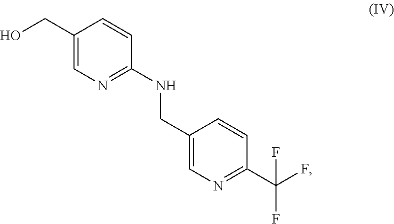 [SMILES: OCc1ccc(NCc2ccc(C(F)(F)F)nc2)nc1]

with a dialkyl carbonate of formula R²O(CO)OR², wherein each R² is a substituted or unsubstituted C₁₋₂₀ straight-chain or C₃₋₂₀ branched-chain alkyl group, in the presence of a base.

In a preferred process of the present invention, the compound of formula (IV) is prepared by reducing a compound of formula (III)

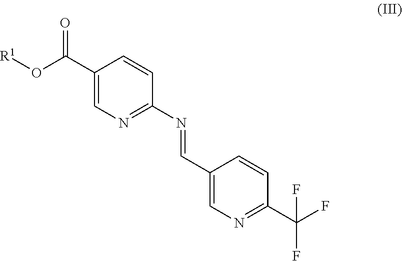

wherein R¹ is a substituted or unsubstituted C₁₋₂₀ straight-chain or C₃₋₂₀ branched-chain alkyl group or a substituted or unsubstituted C₆₋₂₀ aryl group;
with hydrogen in the presence of a Group 8 transition metal catalyst and a base.

In a preferred process of the present invention, the compound of formula (III) is prepared by reacting a compound of formula (I)

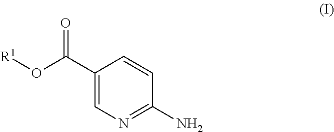

wherein R¹ is as hereinbefore defined,
with a compound of formula (II)

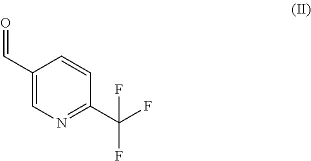 [SMILES: O=Cc1ccc(C(F)(F)F)nc1]

in the presence of an acid catalyst.

In a preferred process of the present invention, the compound of formula (IX) is prepared by reacting a compound of formula (VIII)

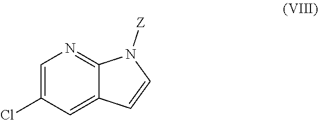

wherein Z is —SO₂R″ or —CO₂R³;
R″ is a substituted or unsubstituted C₁₋₂₀ straight-chain or C₃₋₂₀ branched-chain alkyl group or a substituted or unsubstituted C₆₋₂₀ aryl group; and
R³ is a substituted or unsubstituted C₁₋₂₀ straight-chain or C₃₋₂₀ branched-chain alkyl group or a substituted or unsubstituted C₆₋₂₀ aryl group;
with a borylation agent in the presence of a Group 9 transition metal catalyst.

In a preferred process of the present invention, the compound of formula (VIII) is prepared by reacting a compound of formula (VII)

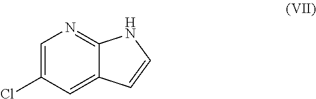

with a sulfonyl halide of formula R″SO₂X^{a} wherein X^{a} is a halide and R″ is as hereinbefore defined, or a compound of formula (R³O(CO))₂O, wherein R³ is as hereinbefore defined.

Thus, viewed from a further aspect, the present invention provides a process for the preparation of a compound of formula (VI), or a salt or derivative thereof,

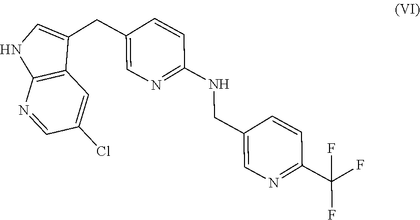 [SMILES: FC(F)(F)c1ccc(CNc2ccc(Cc3c[nH]c4ncc(Cl)cc34)cn2)cn1]

comprising:
reacting a compound of formula (I)

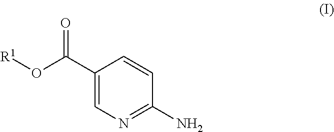

wherein R¹ is a substituted or unsubstituted C₁₋₂₀ straight-chain or C₃₋₂₀ branched-chain alkyl group or a substituted or unsubstituted C₆₋₂₀ aryl group,
with a compound of formula (II),

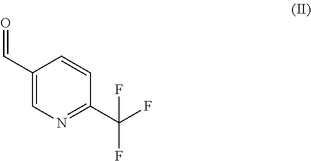 [SMILES: O=Cc1ccc(C(F)(F)F)nc1]

in the presence of an acid catalyst, to give a compound of formula (III),

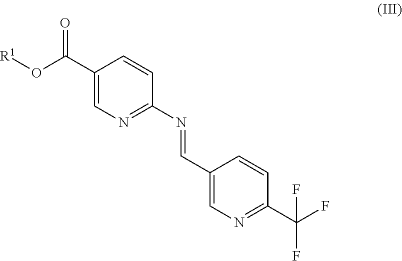

wherein R¹ is as hereinbefore defined;
reducing said compound of formula (III) with hydrogen in the presence of a Group 8 transition metal catalyst and a first base to give a compound of formula (IV)

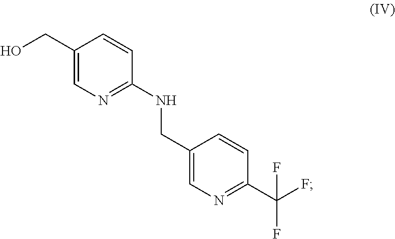 [SMILES: OCc1ccc(NCc2ccc(C(F)(F)F)nc2)nc1]

reacting said compound of formula (IV), or a salt thereof, with a dialkyl carbonate of formula R²O(CO)OR², wherein each R² is a substituted or unsubstituted C₁₋₂₀ straight-chain or C₃₋₂₀ branched-chain alkyl group, in the presence of a second base to give a compound of formula (v)

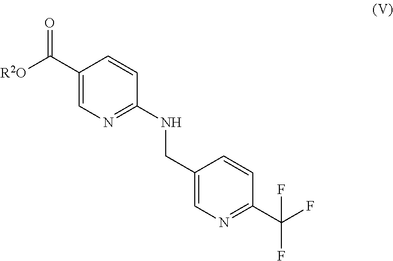

wherein R² is as hereinbefore defined;
reacting a compound of formula (VII)

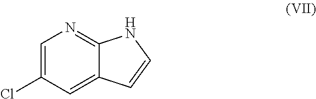

with a sulfonyl halide of formula R″SO₂X^{a}, wherein X^{a} is a halide and R″ is a substituted or unsubstituted C₁₋₂₀ straight-chain or C₃₋₂₀ branched-chain alkyl group or a substituted or unsubstituted C₆₋₂₀ aryl group, or a compound of formula (R³O(CO))₂O, wherein R³ is a substituted or unsubstituted C₁₋₂₀ straight-chain or C₃₋₂₀ branched-chain alkyl group or a substituted or unsubstituted C₆₋₂₀ aryl group, to give a compound of formula (VIII)

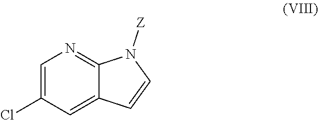

wherein Z is —SO₂R″ or —CO₂R³, and
R″ and R³ are as hereinbefore defined;
reacting said compound of formula (VIII) with a borylation agent in the presence of a Group 9 transition metal catalyst to give a compound of formula (IX)

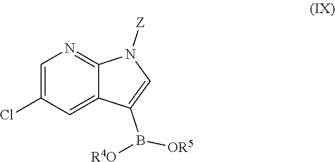

wherein Z is as hereinbefore defined,
R⁴ and R⁵ are, independently, H or a substituted or unsubstituted organic group comprising 1-20 carbon atoms; or, together with the atoms to which they are attached, R⁴ and R⁵ form a ring; and
reacting said compound of formula (V) with said compound of formula (IX) in the presence of a Group 10 transition metal catalyst, a third base, and water to give said compound of formula (VI).

Viewed from a further aspect, the present invention provides a process for the preparation of a compound of formula (VI), or a salt or derivative thereof,

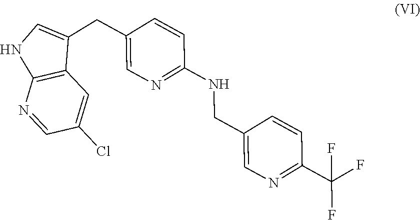 [SMILES: FC(F)(F)c1ccc(CNc2ccc(Cc3c[nH]c4ncc(Cl)cc34)cn2)cn1]

comprising:
reacting a compound of formula (I)

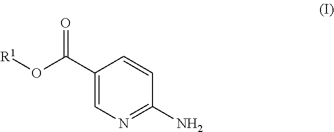

wherein R¹ is a substituted or unsubstituted C₁₋₂₀ straight-chain or C₃₋₂₀ branched-chain alkyl group or a substituted or unsubstituted C₆₋₂₀ aryl group,
with a compound of formula (II),

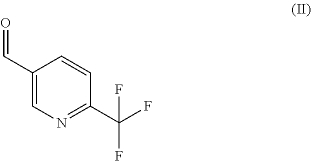 [SMILES: O=Cc1ccc(C(F)(F)F)nc1]

in the presence of an acid catalyst, to give a compound of formula (III),

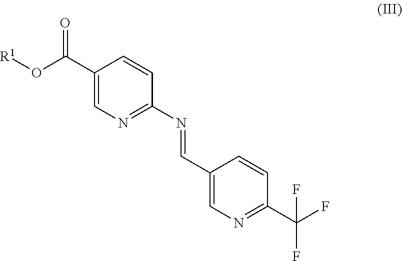

wherein R¹ is as hereinbefore defined;
reducing said compound of formula (III) with hydrogen in the presence of a Group 8 transition metal catalyst and a first base to give a compound of formula (IV)

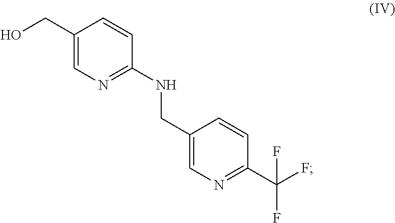 [SMILES: OCc1ccc(NCc2ccc(C(F)(F)F)nc2)nc1]

reacting a compound of formula (VII)

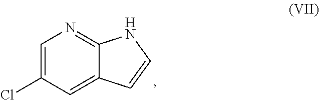

with a sulfonyl halide of formula R″SO₂X^{a}, wherein X^{a} is a halide and R″ is a substituted or unsubstituted C₁₋₂₀ straight-chain or C₃₋₂₀ branched-chain alkyl group or a substituted or unsubstituted C₆₋₂₀ aryl group, or a compound of formula (R³O(CO))₂O, wherein R³ is a substituted or unsubstituted C₁₋₂₀ straight-chain or C₃₋₂₀ branched-chain alkyl group or a substituted or unsubstituted C₆₋₂₀ aryl group, to give a compound of formula (VIII)

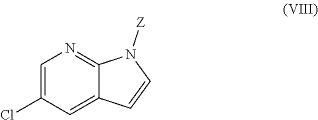

wherein Z is —SO₂R″ or —CO₂R³, and
R″ and R³ are as hereinbefore defined;
reacting said compound of formula (VIII) with a borylation agent in the presence of a Group 9 transition metal catalyst to give a compound of formula (IX)

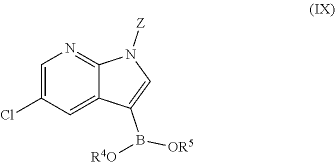

wherein Z is as hereinbefore defined,
R⁴ and R⁵ are, independently, H or a substituted or unsubstituted organic group comprising 1-20 carbon atoms; or, together with the atoms to which they are attached, R⁴ and R⁵ form a ring; and
reacting a compound of formula (V)

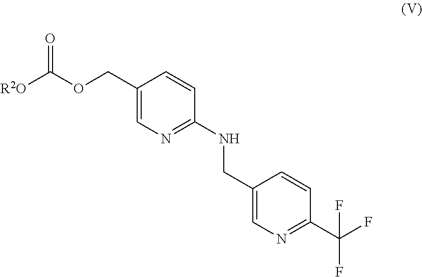

wherein R² is a substituted or unsubstituted C₁₋₂₀ straight-chain or C₃₋₂₀ branched-chain alkyl group,
with said compound of formula (IX) in the presence of a Group 10 transition metal catalyst, a third base, and water to give said compound of formula (VI),
wherein said compound of formula (V) is prepared in situ in the presence of the compound of formula (IX), the Group 10 transition metal catalyst, the third base and water by reacting said compound of formula (IV), or a salt thereof, with a dialkyl carbonate of formula R²O(CO)OR², wherein each R² is a substituted or unsubstituted C₁₋₂₀ straight-chain or C₃₋₂₀ branched-chain alkyl group.

Viewed from a further aspect, the present invention provides a compound of formula (III), or a salt or derivative thereof,

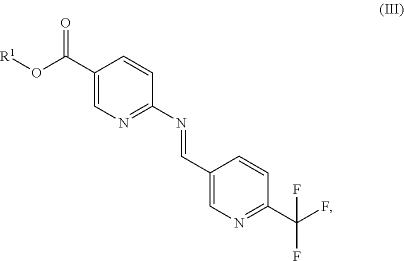

wherein R¹ is a substituted or unsubstituted C₁₋₂₀ straight-chain or C₃₋₂₀ branched-chain alkyl group or a substituted or unsubstituted C₆₋₂₀ aryl group.

Viewed from a further aspect, the present invention provides a compound of formula (V), or a salt or derivative thereof,

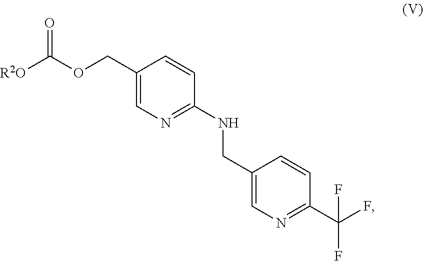

wherein R² is a substituted or unsubstituted C₁₋₂₀ straight-chain or C₃₋₂₀ branched-chain alkyl group.

Viewed from a further aspect, the present invention provides a compound of formula (IX), or a salt or derivative thereof,

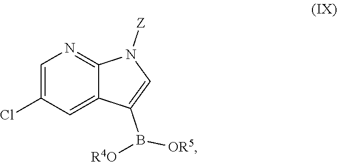

Z is —SO₂R″ or —CO₂R³;
R″ is a substituted or unsubstituted C₁₋₂₀ straight-chain or C₃₋₂₀ branched-chain alkyl group or a substituted or unsubstituted C₆₋₂₀ aryl group;
R³ is a substituted or unsubstituted C₁₋₂₀ straight-chain or C₃₋₂₀ branched-chain alkyl group or a substituted or unsubstituted C₆₋₂₀ aryl group; and
R⁴ and R⁵ are, independently, H or a substituted or unsubstituted organic group comprising 1-20 carbon atoms; or, together with the atoms to which they are attached, R⁴ and R⁵ form a ring;
provided that Z is not tosyl.

Viewed from a further aspect, the present invention provides a pharmaceutical composition comprising a compound as hereinbefore described.

Viewed from a further aspect, the present invention also provides compounds and compositions as hereinbefore described for use as a medicament.

Viewed from a further aspect, the present invention also provides compounds and compositions as hereinbefore described for use in the treatment of cancer, preferably wherein the cancer is tenosynovial giant cell tumor (TGCT).

Viewed from a further aspect, the present invention also provides the use of compounds and compositions as hereinbefore described for the manufacture of a medicament for the treatment of cancer, preferably wherein the cancer is tenosynovial giant cell tumor (TGCT).

### DEFINITIONS

The point of attachment of a moiety or substituent is represented by “—”. For example, —OH is attached through the oxygen atom.

As used herein, the term “alkyl” refers to a straight-chain or branched saturated hydrocarbon group. In certain embodiments, the alkyl group may have from 1-20 carbon atoms, in certain embodiments from 1-15 carbon atoms, in certain embodiments, 1-8 carbon atoms. The alkyl group may be unsubstituted. Alternatively, the alkyl group may be substituted. Unless otherwise specified, the alkyl group may be attached at any suitable carbon atom and, if substituted, may be substituted at any suitable atom. Typical alkyl groups include but are not limited to methyl, ethyl, n-propyl, iso-propyl, n-butyl, iso-butyl, sec-butyl, tert-butyl, n-pentyl, n-hexyl and the like.

As used herein, the term “cycloalkyl” refers to a saturated carbocyclic hydrocarbon radical. The cycloalkyl group may have a single ring or multiple condensed rings. In certain embodiments, the cycloalkyl group may have from 3-15 carbon atoms, in certain embodiments, from 3-10 carbon atoms, in certain embodiments, from 3-8 carbon atoms. The cycloalkyl group may be unsubstituted. Alternatively, the cycloalkyl group may be substituted. Unless other specified, the cycloalkyl group may be attached at any suitable carbon atom and, if substituted, may be substituted at any suitable atom. Typical cycloalkyl groups include but are not limited to cyclopropyl, cyclobutyl, cyclopentyl, cyclohexyl and the like.

As used herein, the term “alkenyl” refers to a straight-chain or branched unsaturated hydrocarbon group comprising at least one carbon-carbon double bond.

As used herein, the term “aryl” refers to an aromatic carbocyclic group. The aryl group may have a single ring or multiple condensed rings. In certain embodiments, the aryl group can have from 6-20 carbon atoms, in certain embodiments from 6-15 carbon atoms, in certain embodiments, 6-12 carbon atoms. The aryl group may be unsubstituted. Alternatively, the aryl group may be substituted. Unless otherwise specified, the aryl group may be attached at any suitable carbon atom and, if substituted, may be substituted at any suitable atom. Examples of aryl groups include, but are not limited to, phenyl, naphthyl, anthracenyl and the like.

As used herein, the term “alkoxy” refers to an optionally substituted group of the formula alkyl-O— or cycloalkyl-O—, wherein alkyl and cycloalkyl are as defined above.

As used herein, the term “arylalkyl” refers to an optionally substituted group of the formula aryl-alkyl, where aryl and alkyl are as defined above.

As used herein, the term “heteroalkyl” refers to a straight-chain or branched saturated hydrocarbon group wherein one or more carbon atoms are independently replaced with one or more heteroatoms (e.g. nitrogen, oxygen, phosphorus and/or sulfur atoms). The heteroalkyl group may be unsubstituted. Alternatively, the heteroalkyl group may be substituted. Unless otherwise specified, the heteroalkyl group may be attached at any suitable atom and, if substituted, may be substituted at any suitable atom. Examples of heteroalkyl groups include but are not limited to ethers, thioethers, primary amines, secondary amines, tertiary amines and the like.

As used herein, the term “heterocycloalkyl” refers to a saturated cyclic hydrocarbon group wherein one or more carbon atoms are independently replaced with one or more heteroatoms (e.g. nitrogen, oxygen, phosphorus and/or sulfur atoms). The heterocycloalkyl group may be unsubstituted. Alternatively, the heterocycloalkyl group may be substituted. Unless otherwise specified, the heterocycloalkyl group may be attached at any suitable atom and, if substituted, may be substituted at any suitable atom. Examples of heterocycloalkyl groups include but are not limited to epoxide, morpholinyl, piperadinyl, piperazinyl, thirranyl, pyrrolidinyl, pyrazolidinyl, imidazolidinyl, thiazolidinyl, thiomorpholinyl and the like.

As used herein, the term “heteroaryl” refers to an aromatic carbocyclic group wherein one or more carbon atoms are independently replaced with one or more heteroatoms (e.g. nitrogen, oxygen, phosphorus and/or sulfur atoms). The heteroaryl group may be unsubstituted. Alternatively, the heteroaryl group may be substituted. Unless otherwise specified, the heteroaryl group may be attached at any suitable atom and, if substituted, may be substituted at any suitable atom. Examples of heteroaryl groups include but are not limited to thienyl, furanyl, pyrrolyl, imidazolyl, pyrazolyl, thiazolyl, isothiazolyl, oxazolyl, isoxazolyl, triazolyl, thiadiazolyl, thiophenyl, oxadiazolyl, pyridinyl, pyrimidyl, benzoxazolyl, benzthiazolyl, benzimidazolyl, indolyl, quinolinyl and the like.

As used herein, the term “substituted” refers to a group in which one or more hydrogen atoms are each independently replaced with substituents (e.g. 1, 2, 3, 4, 5 or more) which may be the same or different. Examples of substituents include but are not limited to -halo, —C(halo)₃, —R^{a}, ═O, ═S, —O—R^{a}, —S—R^{a}, —NR^{a}R^{b}, —CN, —NO₂, —C(O)—R^{a}, —COOR^{a}, —C(S)—R^{a}, —C(S)OR^{a}, —S(O)₂OH, —S(O)₂—R^{a}, —S(O)₂NR^{a}R^{b}, —O—S(O)—R^{a} and —CONR^{a}R^{b}, such as -halo, —C(halo)₃ (e.g. —CF₃), —R^{a}, —O—R^{a}, —NR^{a}R^{b}, —PR^{a}R^{b}R^{c}, —CN, or —NO₂. R^{a}, R^{b} and R^{c} are independently selected from the groups consisting of H, alkyl, aryl, arylalkyl, heteroalkyl, heteroaryl, or R^{a} and R^{b} together with the atom to which they are attached form a heterocycloalkyl group. R^{a}, R^{b} and R^{c} may be unsubstituted or further substituted as defined herein.

As used herein, the term “coupling” refers to a chemical reaction in which two molecules or parts of a molecule join together (Oxford Dictionary of Chemistry, Sixth Edition, 2008).

As used herein, the terms “halo” or “hal” refer to —F, —Cl, —Br and —I.

As used herein, the term “Ru-SNS” refers to dichlorotriphenylphosphine[bis(2-(ethylthio)ethyl)amine]ruthenium(II).

As used herein, the term “dippf” refers to 1,1′-bis(diisopropylphosphino)ferrocene.

As used herein, the term “Bpin” refers to (pinacolato)boron.

As used herein, the term “HBpin” refers to pinacolborane.

As used herein, the term “B₂pin₂” refers to bis(pinacolato)diboron.

As used herein, the term “Bcat” refers to (catecholato)boron.

As used herein, the term “HBcat” refers to catecholborane.

As used herein, the term “DABCO” refers to 1,4-diazabicyclo[2.2.2]octane.

As used herein, the term “DMAP” refers to 4-dimethylaminopyridine.

As used herein, the term “DBU” refers to 1,8-diazabicyclo[5.4.0]undec-7-ene.

As used herein, the term “COD” refers to cyclooctadiene.

As used herein, the term “room temperature” refers to a temperature in the range 15 to 35° C.

As used herein, the term “Group 8 transition metal” refers to a transition metal in group 8 of the periodic table.

As used herein, the term “Group 9 transition metal” refers to a transition metal in group 9 of the periodic table.

As used herein, the term “Group 10 transition metal” refers to a transition metal in group 10 of the periodic table.

### DETAILED DESCRIPTION

Preferred and/or optional features of the invention will now be set out. Any aspect of the invention may be combined with any other aspect of the invention, unless the context demands otherwise. Any of the preferred or optional features of any aspect of the invention may be combined, singly or in combination, with any aspect of the invention, unless the context demands otherwise.

The present invention provides a process for the preparation of pexidartinib, or a salt or derivative thereof. The process involves the use of low-cost and readily available starting materials, and has a reduced overall step count compared to known processes. Additionally, the steps of the process of the present invention either minimise the use of toxic and/or corrosive reagents or avoid them completely.

The present invention is directed to a process for the preparation of a compound of formula (VI), or a salt or derivative thereof,

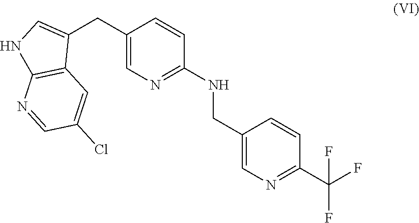 [SMILES: FC(F)(F)c1ccc(CNc2ccc(Cc3c[nH]c4ncc(Cl)cc34)cn2)cn1]

comprising reacting a compound of formula (V)

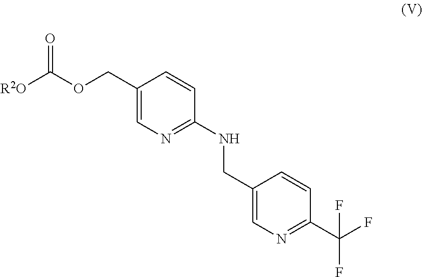

wherein R² is a substituted or unsubstituted C₁₋₂₀ straight-chain or C₃₋₂₀ branched-chain alkyl group,
with a compound of formula (IX)

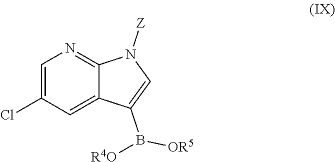

wherein
Z is —SO₂R″ or —CO₂R³;
R″ is a substituted or unsubstituted C₁₋₂₀ straight-chain or C₃₋₂₀ branched-chain alkyl group or a substituted or unsubstituted C₆₋₂₀ aryl group;
R³ is a substituted or unsubstituted C₁₋₂₀ straight-chain or C₃₋₂₀ branched-chain alkyl group or a substituted or unsubstituted C₆₋₂₀ aryl group; and
R⁴ and R⁵ are, independently, H or a substituted or unsubstituted organic group comprising 1-20 carbon atoms; or, together with the atoms to which they are attached, R⁴ and R⁵ form a ring;
in the presence of a Group 10 transition metal catalyst, a base, and water to give said compound of formula (VI).

The processes of the present invention employ a Group 10 transition metal catalyst. More preferably, the Group 10 transition metal catalyst is a nickel catalyst, a palladium catalyst or a platinum catalyst. More preferably, the Group 10 transition metal catalyst is a palladium catalyst or a platinum catalyst. Most preferably, the Group 10 transition metal catalyst is a palladium catalyst.

In preferred processes of the present invention, the Group 10 transition metal catalyst is of formula (A)

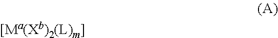

wherein,
M^{a} is a Group 10 transition metal, preferably palladium;
X^{b} is an anionic ligand;
L is a monodentate phosphorus ligand, or a bidentate phosphorus ligand;
m is 1 or 2, wherein,
when m is 1, L is a bidentate phosphorus ligand; and
when m is 2, each L is a monodentate phosphorus ligand.

In preferred Group 10 transition metal catalysts of formula (A), X^{b} is a halo group, such as —Cl, —Br, and —I, or trifluoroacetate (i.e. F₃CCO₂⁻). Preferably, X^{b} is —Cl.

In the Group 10 transition metal catalysts of formula (A), L is a monodentate phosphorus ligand, or a bidentate phosphorus ligand. Any suitable phosphorus compound capable of forming a ligand-metal interaction with the M^{a} atom may be used. In the ligand, each phosphorus atom is covalently bonded to either 3 carbon atoms (tertiary phosphines) or to x heteroatoms and 3−x carbon atoms, where x=1, 2 or 3. Preferably, the heteroatom is selected from the group consisting of N and O.

The phosphorus ligand L may be monodentate, e.g. PPh₃, or bidentate.

The phosphorous ligand L may be chiral or achiral.

The phosphorous ligand L may be substituted or unsubstituted.

Phosphorus ligands L that may be used in the present invention include but are not restricted to the following structural types:

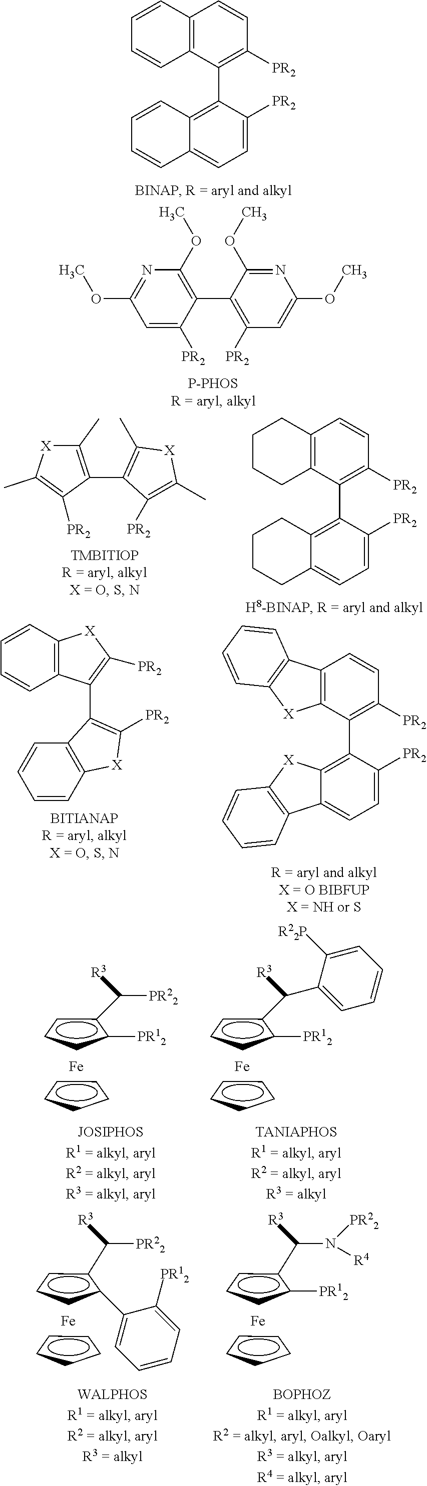

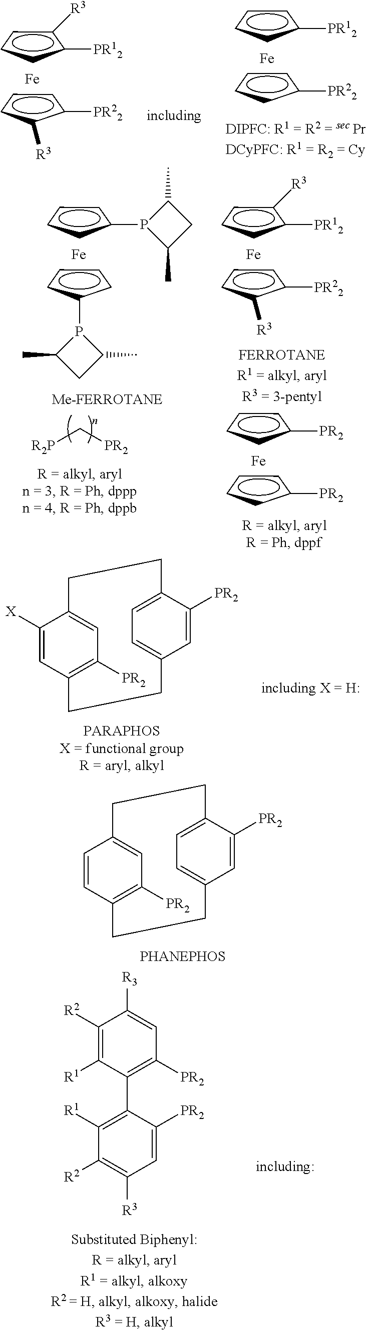

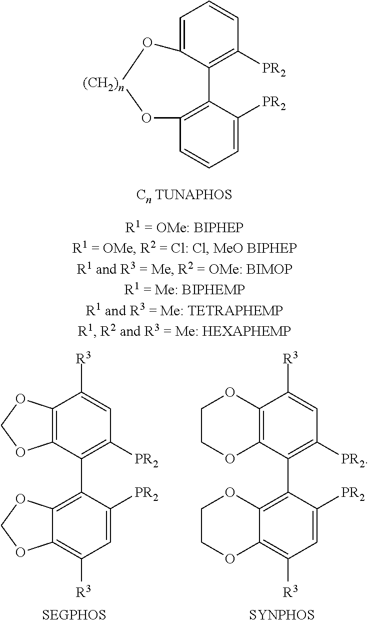

In the above structures —PR₂ may be —P(alkyl)₂ in which alkyl is preferably C₁-C₁₀ alkyl, —P(aryl)₂ where aryl includes phenyl and naphthyl which may be substituted or unsubstituted or —P(O-alkyl)₂ and —P(O-aryl)₂ with alkyl and aryl as defined above. —PR₂ may also be substituted or unsubstituted —P(heteroaryl)₂, where heteroaryl includes furanyl (e.g. 2-furanyl or 3-furanyl). —PR₂ is preferably either —P(aryl)₂ where aryl includes phenyl, tolyl, xylyl or anisyl or —P(O-aryl)₂. If —PR₂ is —P(O-aryl)₂, the most preferred O-aryl groups are those based on chiral or achiral substituted 1,1′-biphenol and 1,1′-binapthol. Alternatively, the R groups on the P-atom may be linked as part of a cyclic structure.

Substituting groups may be present on the alkyl or aryl substituents in the phosphorus ligands. Such substituting groups are typically branched or linear C₁₋₆ alkyl groups such as methyl, ethyl, propyl, iso-propyl, and tert-butyl.

These phosphorus ligands depicted above are generally available commercially and their preparation is known.

Preferred bidentate phosphorus ligands L include Binap ligands, PPhos ligands, PhanePhos ligands, Josiphos ligands and Bophoz ligands, preferably Binap ligands.

Preferred bidentate phosphorus ligands L are also ligands of formula R⁶R⁷P(CH₂)ₙPR⁸R⁹, wherein n is an integer selected from 1 to 10, preferably 2 to 6 (e.g. 3 or 4) and R⁶, R⁷, R⁸, and R⁹ may be independently selected from the group consisting of unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₆₋₂₀-aryl, substituted C₆₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl. R⁶, R⁷, R⁸, and R⁹ may be independently substituted or unsubstituted branched- or straight-chain alkyl groups such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In one embodiment, the alkyl groups may be optionally substituted with one or more substituents such as halide (F, Cl, Br or I) or alkoxy groups, e.g. methoxy, ethoxy or propoxy. The aryl group may be optionally substituted with one or more (e.g. 1, 2, 3, 4, or 5) substituents such as halide (—F, —Cl, —Br or —I), straight- or branched-chain C₁-C₁₀-alkyl (e.g. methyl), C₁-C₁₀ alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—). Substituted or unsubstituted heteroaryl groups such as pyridyl may also be used. In an alternative embodiment, R⁶ and R⁷ and/or R⁸ and R⁹ may be linked to form a ring structure with the phosphorus atom, preferably 4- to 7-membered rings. Preferably, the bidentate phosphorous ligand is selected from dppm (1,3-bis(diphenylphosphino)methane), dppe (1,3-bis(diphenylphosphino)ethane), dppp (1,3-bis(diphenylphosphino)propane), dppb (1,4-bis(diphenylphosphino)butane), 1,3-bis(diphenylphosphino)pentane, and 1,3-bis(diphenylphosphino)hexane, more preferably dppp and dppb.

Preferred bidentate phosphorus ligands L are also ligands of formula (A1)

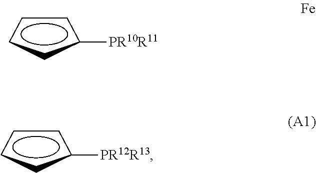

wherein R¹⁰, R¹¹, R¹², and R¹³ may be independently selected from the group consisting of unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₆₋₂₀-aryl, substituted C₆₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl. R¹⁰, R¹¹, R¹², and R¹³ may be independently substituted or unsubstituted branched- or straight-chain alkyl groups such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In one embodiment, the alkyl groups may be optionally substituted with one or more substituents such as halide (F, Cl, Br or I) or alkoxy groups, e.g. methoxy, ethoxy or propoxy. The aryl group may be optionally substituted with one or more (e.g. 1, 2, 3, 4, or 5) substituents such as halide (—F, —Cl, —Br or —I), straight- or branched-chain C₁-C₁₀-alkyl (e.g. methyl), C₁-C₁₀ alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—). Substituted or unsubstituted heteroaryl groups such as pyridyl may also be used. Preferably, the bidentate ligand is selected from dppf (1,1′-bis(diphenylphosphino)ferrocene), dippf (1,1′-bis(di-isopropylphosphino)ferrocene), dCyPfc (1,1′-bis(di-cyclohexylphosphino)ferrocene and dtbpf (1,1′-bis(di-tert-butylphosphino)ferrocene), more preferably dippf and dCyPfc.

Preferred monodentate phosphorous ligands L are tertiary phosphine ligands of the formula PR¹⁴R¹⁵R¹⁶, R¹⁴, R¹⁵ and R¹⁶ may be independently selected from the group consisting of unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₆₋₂₀-aryl, substituted C₆₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl. R¹⁴, R¹⁵ and R¹⁶ may be independently substituted or unsubstituted branched- or straight-chain alkyl groups such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In one embodiment, the alkyl groups may be optionally substituted with one or more substituents such as halide (F, Cl, Br or I) or alkoxy groups, e.g. methoxy, ethoxy or propoxy. The aryl group may be optionally substituted with one or more (e.g. 1, 2, 3, 4, or 5) substituents such as halide (—F, —Cl, —Br or —I), straight- or branched-chain C₁-C₁₀-alkyl (e.g. methyl), C₁-C₁₀ alkoxy, straight- or branched-chain C₁-C₁₀-(dialkyl)amino, C₃₋₁₀ heterocycloalkyl groups (such as morpholiny) and piperadinyl) or tri(halo)methyl (e.g. F₃C—). Substituted or unsubstituted heteroaryl groups such as pyridyl may also be used. In an alternative embodiment, any two of R¹⁴, R¹⁵ and R¹⁶ may be linked to form a ring structure with the phosphorus atom, preferably 4- to 7-membered rings. Preferably, R¹⁴, R¹⁵ and R¹⁶ are the same and are phenyl, i.e. PR¹⁴R¹⁵R¹⁶ is triphenylphosphine. Alternatively, R¹⁴, R¹⁵ and R¹⁶ may be the same and are tolyl, i.e. PR¹⁴R¹⁵R¹⁶ is tritolylphosphine (e.g. ortho, meta- or para-tritolylphosphine). Alternatively, R¹⁴, R¹⁵ and R¹⁶ are the same and are cyclohexyl, i.e. PR¹⁴R¹⁵R¹⁶ is tricyclohexylphosphine. Alternatively, R¹⁴, R¹⁵ and R¹⁶ are the same and are tert-butyl, i.e. PR¹⁴R¹⁵R¹⁶ is tri(tert-butyl)phosphine.

Preferred phosphorus ligands L are selected from the group consisting of PPh₃, tritolylphosphine, PCy₃ (tricyclohexylphosphine), P^{t}Bu₃, dppm (1,3-bis(diphenylphosphino)methane), dppe (1,3-bis(diphenylphosphino)ethane), dppp (1,3-bis(diphenylphosphino)propane), dppb (1,4-bis(diphenylphosphino)butane), 1,3-bis(diphenylphosphino)pentane, 1,3-bis(diphenylphosphino)hexane, dppf (1,1′-bis(diphenylphosphino)ferrocene), dippf (1,1′-bis(di-isopropylphosphino)ferrocene), dCyPfc (1,1′-bis(di-cyclohexylphosphino)ferrocene and dtbpf (1,1′-bis(di-tert-butylphosphino)ferrocene).

Particularly preferred phosphorus ligands L are selected from the group consisting of dppp, dppb, dppf, dippf, dtbpf and dCyPfc, more preferably dippf, dtbpf and dCyPfc, even more preferably dippf.

In preferred processes of the present invention, the Group 10 transition metal catalyst is selected from PdX^{b}₂(dppp), PdX^{b}₂(dppb), PdX^{b}₂(dppf), PdX^{b}₂(dippf), PdX^{b}₂(dtbpf) and PdX^{b}₂(dCyPfc) wherein X^{b} is as defined above, more preferably PdX^{b}₂(dippf), PdX^{b}₂(dtbpf) and PdX^{b}₂(dCyPfc), even more preferably PdX^{b}₂(dippf).

In preferred processes of the present invention, the Group 10 transition metal catalyst is selected from PdCl₂(dppp), PdCl₂(dppb), PdCl₂(dppf), PdCl₂(dippf), PdCl₂(dtbpf) and PdCl₂(dCyPfc), more preferably PdCl₂(dippf), PdCl₂(dtbpf) and PdCl₂(dCyPfc), even more preferably PdCl₂(dippf).

In alternative preferred processes of the present invention, the Group 10 transition metal catalyst is of formula (B)

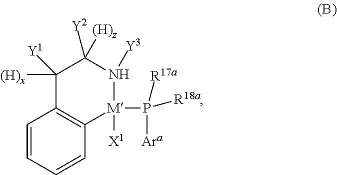

wherein,
M′ is a Group 10 transition metal, preferably palladium;
X¹ is an anionic ligand;
Ar^{a} is a substituted or unsubstituted aryl group, a substituted or unsubstituted heteroaryl group, or a substituted or unsubstituted xanthenyl group;
R^{17a} and R^{18b} are independently organic groups having 1-20 carbon atoms, or R^{17a} and R^{18a} are linked to form a ring structure with P;
each of Y¹ and Y² is hydrogen; or together with the atoms to which they are attached, Y¹ and Y² form an aromatic ring;
Y³ is hydrogen, a substituted or unsubstituted C₁₋₂₀ straight-chain or C₃₋₂₀ branched-chain alkyl group or a substituted or unsubstituted C₆₋₂₀ aryl group;
x and z are 0 or 1, wherein
when x and z are both 1, each of Y¹ and Y² is hydrogen; and
when x and z are both 0, together with the atoms to which they are attached, Y¹ and Y² form an aromatic ring.

In the Group 10 transition metal catalysts of formula (B), X¹ is an anionic ligand. X¹ may be a coordinated anionic ligand or a non-coordinated anionic ligand.

In the Group 10 transition metal catalysts of formula (B), X¹ is preferably a halo group, such as —Cl, —Br, and —I, or mesylate (i.e. MSO⁻ or MeSO₃⁻). More preferably, X¹ is —Cl.

In preferred Group 10 transition metal catalysts of formula (B), each of Y¹ and Y² is hydrogen. In this instance, x and z are both 1.

In alternative preferred Group 10 transition metal catalysts of formula (B), together with the atoms to which they are attached, Y¹ and Y² form an aromatic ring. In this instance, x and z are both 0. Preferably, the aromatic ring is a six-membered aromatic ring. More preferably, together with the atoms to which they are attached, Y¹ and Y² form a benzene ring.

In the Group 10 transition metal catalysts of formula (B), Y³ is preferably hydrogen, a substituted or unsubstituted C₁₋₁₀ straight-chain or C₃₋₁₀ branched-chain alkyl group or a substituted or unsubstituted C₆₋₁₀ aryl group. More preferably, Y³ is hydrogen, a substituted or unsubstituted C₁₋₅ straight-chain or C₃₋₅ branched-chain alkyl group or a substituted or unsubstituted C₆₋₁₀ aryl group. Most preferably, Y³ is hydrogen, methyl or phenyl.

In the Group 10 transition metal catalysts of formula (B), R^{17a} and R^{18a} may be the same or different. In one embodiment, R^{17a} and R^{18a} are the same. In another embodiment, R^{17a} and R^{18a} are different. R^{17a} and R^{18a} are selected up to the limitations imposed by stability and the rules of valence. R^{17a} and R^{18a} may be independently selected from the group consisting of substituted and unsubstituted straight-chain C₁₋₂₀-alkyl, substituted and unsubstituted branched-chain C₃₋₂₀-alkyl, substituted and unsubstituted C₃₋₂₀-cycloalkyl, substituted and unsubstituted C₆₋₂₀-aryl, and substituted and unsubstituted C₄₋₂₀-heteroaryl wherein the heteroatoms are independently selected from sulfur, nitrogen and oxygen. R^{17a} and R^{18a} may independently be substituted or unsubstituted branched- or straight-chain alkyl groups such as methyl, ethyl, n-propyl, iso-propyl, n-butyl, iso-butyl, sec-butyl, tert-butyl, pentyl (e.g. n-pentyl or neopentyl), hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In one embodiment, the alkyl groups may be optionally substituted with one or more (e.g. 1, 2, 3, 4, or 5) substituents each of which may be the same or different such as halide (F, Cl, Br or I) or alkoxy groups, e.g. methoxy, ethoxy or propoxy. The aryl group may be optionally substituted with one or more (e.g. 1, 2, 3, 4, or 5) substituents each of which may be the same or different such as halide (F, Cl, Br or I), straight- or branched-chain alkyl (e.g. C₁-C₁₀), alkoxy (e.g. C₁-C₁₀ alkoxy), straight- or branched-chain (dialkyl)amino (e.g. C₁-C₁₀ dialkyl)amino), heterocycloalkyl (e.g. C₃₋₁₀ heterocycloalkyl groups, such as morpholinyl and piperadinyl) or tri(halo)methyl (e.g. F₃C—). Suitable substituted aryl groups include but are not limited to 4-dimethylaminophenyl, 4-methylphenyl, 3,5-dimethylphenyl, 4-methoxyphenyl, 4-methoxy-3,5-dimethylphenyl and 3,5-di(trifluoromethyl)phenyl. Substituted or unsubstituted heteroaryl groups such as pyridyl may also be used. In an alternative embodiment, R^{17a} and R^{18a} are linked to form a ring structure with P, preferably 4- to 7-membered rings. Preferably, R^{17a} and R^{18a} are the same and are tert-butyl, cyclohexyl, adamantyl, phenyl or substituted phenyl groups, such as 3,5-di(trifluoromethyl)phenyl.

In the Group 10 transition metal catalysts of formula (B), Ar^{a} is a substituted or unsubstituted aryl group, a substituted or unsubstituted heteroaryl group, or a substituted or unsubstituted xanthenyl group. Preferably, Ar^{a} is a substituted or unsubstituted phenyl group, a substituted or unsubstituted biphenyl group, a substituted or unsubstituted napthyl group, a substituted or unsubstituted binapthyl group, a substituted or unsubstituted pyrazolyl group, a substituted or unsubstituted bipyrazolyl group, or a substituted or unsubstituted xanthenyl group. More preferably, Ar^{a} is a substituted or unsubstituted biphenyl group, a substituted or unsubstituted binapthyl group, a substituted or unsubstituted bipyrazolyl group, or a substituted or unsubstituted xanthenyl group. Even more preferably, Ar^{a} is a substituted or unsubstituted biphenyl group, a substituted or unsubstituted bipyrazolyl group, or a substituted or unsubstituted xanthenyl group.

In the Group 10 transition metal catalysts of formula (B), the phosphine ligand —P(R^{17a})(R^{18b})Ar^{a} is preferably selected from the group consisting of:

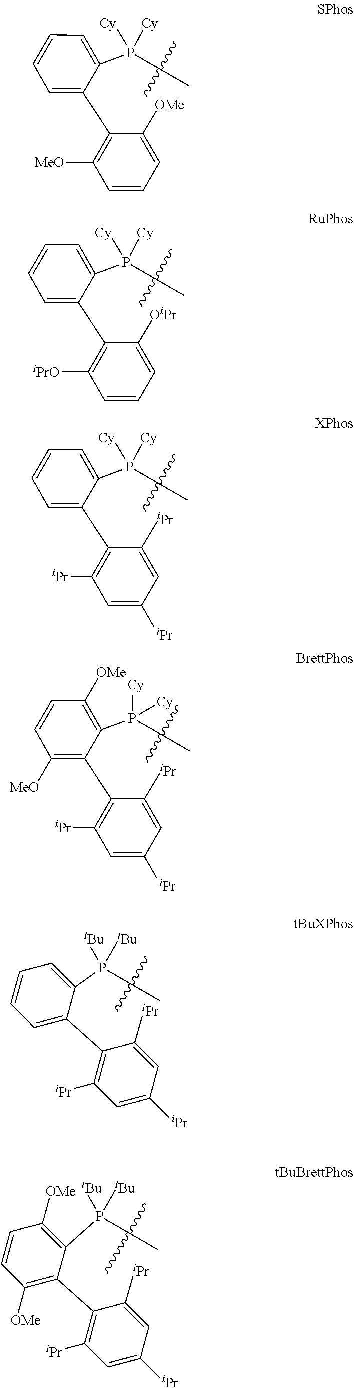

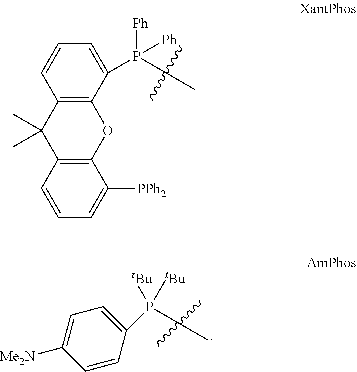

In preferred processes of the present invention, the Group 10 transition metal catalyst is selected from:

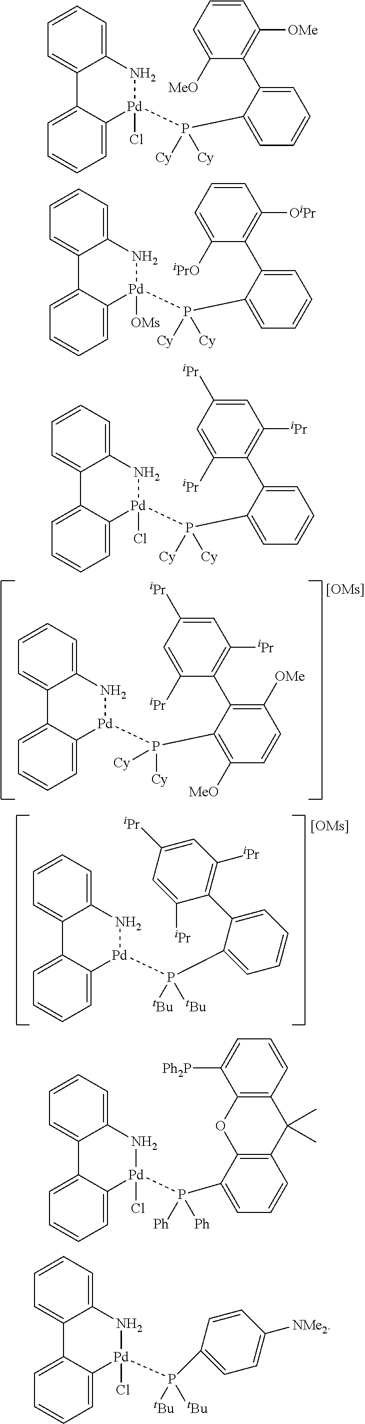

In alternative preferred processes of the present invention, the Group 10 transition metal catalyst is of formula (Ca)

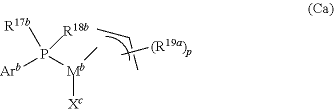

wherein,
M^{b} is a Group 10 transition metal, preferably palladium;
X^{c} is a coordinated anionic ligand;
Ar^{b} is a substituted or unsubstituted aryl group or a substituted or unsubstituted heteroaryl group;
R^{17b} and R^{18b} are independently organic groups having 1-20 carbon atoms, or R^{17b} and R^{18b} are linked to form a ring structure with P;
R^{19a} is an organic group having 1-20 carbon atoms; and
p is 0, 1, 2, 3, 4 or 5.

In the Group 10 transition metal catalysts of formula (Ca), X^{c} is a coordinated anionic ligand i.e. the anionic ligand is bonded to the Pd atom within the coordination sphere. X^{c} is preferably a halo group, such as —Cl, —Br, and —I, or trifluoroacetate (i.e. F₃CCO₂⁻). More preferably, X^{c} is —Cl.

In the Group 10 transition metal catalysts of formula (Ca), R^{17b} and R^{18b} may be the same or different. In one embodiment, R^{17b} and R^{18b} are the same. In another embodiment, R^{17b} and R^{18b} are different. R^{17b} and R^{18b} are selected up to the limitations imposed by stability and the rules of valence. R^{17b} and R^{18b} may be independently selected from the group consisting of substituted and unsubstituted straight-chain C₁₋₂₀-alkyl, substituted and unsubstituted branched-chain C₃₋₂₀-alkyl, substituted and unsubstituted C₃₋₂₀-cycloalkyl, substituted and unsubstituted C₆₋₂₀-aryl, and substituted and unsubstituted C₄₋₂₀-heteroaryl wherein the heteroatoms are independently selected from sulfur, nitrogen and oxygen. R^{17b} and R^{18b} may independently be substituted or unsubstituted branched- or straight-chain alkyl groups such as methyl, ethyl, n-propyl, iso-propyl, n-butyl, iso-butyl, sec-butyl, tert-butyl, pentyl (e.g. n-pentyl or neopentyl), hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In one embodiment, the alkyl groups may be optionally substituted with one or more (e.g. 1, 2, 3, 4, or 5) substituents each of which may be the same or different such as halide (F, Cl, Br or I) or alkoxy groups, e.g. methoxy, ethoxy or propoxy. The aryl group may be optionally substituted with one or more (e.g. 1, 2, 3, 4, or 5) substituents each of which may be the same or different such as halide (F, Cl, Br or I), straight- or branched-chain alkyl (e.g. C₁-C₁₀), alkoxy (e.g. C₁-C₁₀ alkoxy), straight- or branched-chain (dialkyl)amino (e.g. C₁-C₁₀ dialkyl)amino), heterocycloalkyl (e.g. C₃₋₁₀ heterocycloalkyl groups, such as morpholinyl and piperadinyl) or tri(halo)methyl (e.g. F₃C—). Suitable substituted aryl groups include but are not limited to 4-dimethylaminophenyl, 4-methylphenyl, 3,5-dimethylphenyl, 4-methoxyphenyl, 4-methoxy-3,5-dimethylphenyl and 3,5-di(trifluoromethyl)phenyl. Substituted or unsubstituted heteroaryl groups such as pyridyl may also be used. In an alternative embodiment, R^{17b} and R^{18b} are linked to form a ring structure with P, preferably 4- to 7-membered rings. Preferably, R^{17b} and R^{18b} are the same and are tert-butyl, cyclohexyl, phenyl or substituted phenyl groups, such as 3,5-di(trifluoromethyl)phenyl. Alternatively, R^{17b} and R^{18b} are independently selected from the group consisting of —Me, —Et, —ⁿPr, —ⁱPr, —ⁿBu, —ⁱBu, cyclohexyl and cycloheptyl.

In the Group 10 transition metal catalysts of formula (Ca), Ar^{b} is a substituted or unsubstituted aryl group, or a substituted or unsubstituted heteroaryl group. Preferably, Ar^{b} is a substituted or unsubstituted phenyl group, a substituted or unsubstituted biphenyl group, a substituted or unsubstituted napthyl group, a substituted or unsubstituted binapthyl group, a substituted or unsubstituted pyrazolyl group, or a substituted or unsubstituted bipyrazolyl group. More preferably, Ar^{b} is a substituted or unsubstituted biphenyl group, a substituted or unsubstituted binapthyl group, or a substituted or unsubstituted bipyrazolyl group. Even more preferably, Ar^{b} is a substituted or unsubstituted biphenyl group, or a substituted or unsubstituted bipyrazolyl group.

In the Group 10 transition metal catalysts of formula (Ca), the phosphine ligand —P(R^{17b})(R^{18b})Ar^{b} is preferably selected from the group consisting of:

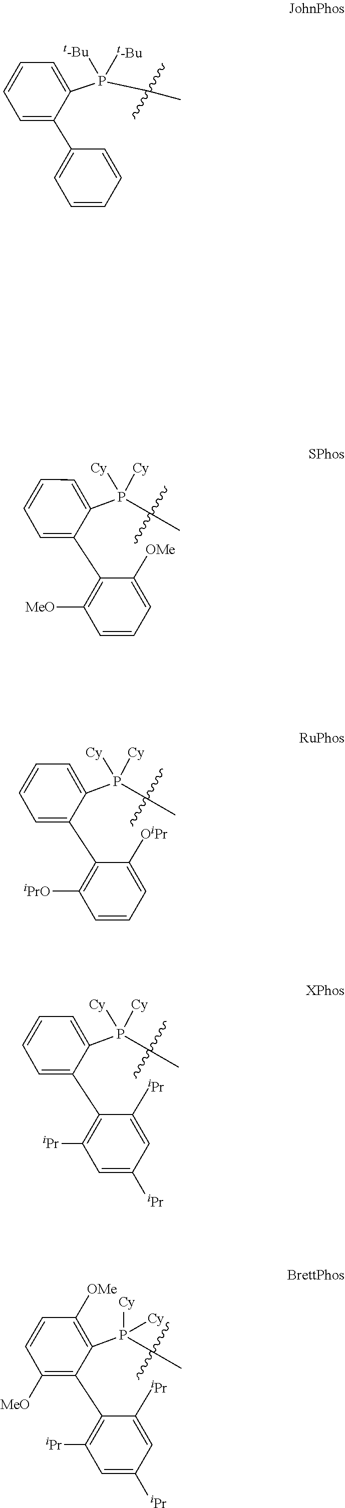

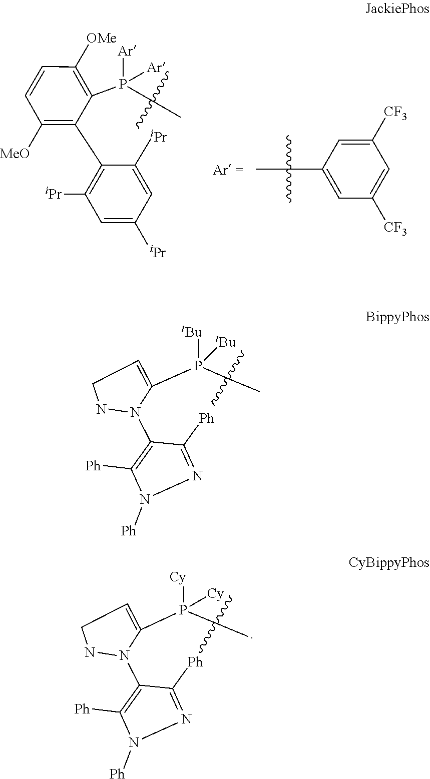

The metal atom in the Group 10 transition metal catalysts of formula (Ca) is coordinated to an optionally substituted allyl group. R^{19a} is an organic group having 1-20 carbon atoms, preferably 1-10 carbon atoms and more preferably 1-8 carbon atoms. R^{19a} is selected up to the limitations imposed by stability and the rules of valence. The number of R^{19a} groups ranges from 0 to 5 i.e. p is 0, 1, 2, 3, 4 or 5. When p is 2, 3, 4 or 5, each of R^{19a} may be the same or different. In certain embodiments, when p is 2, 3, 4, or 5, each R^{19a} is the same. In certain embodiments, p is 0 (i.e. the allyl group is unsubstituted). In certain embodiments, p is 1. In certain embodiments, p is 2, wherein each R^{19a} is the same or different.

In the Group 10 transition metal catalysts of formula (Ca), R^{19a} may be selected from the group consisting of substituted and unsubstituted straight-chain C₁₋₂₀-alkyl, substituted and unsubstituted branched-chain C₃₋₂₀-alkyl, substituted and unsubstituted C₃₋₂₀-cycloalkyl, substituted and unsubstituted C₆₋₂₀-aryl, and substituted and unsubstituted C₄₋₂₀-heteroaryl wherein the heteroatoms are independently selected from sulfur, nitrogen and oxygen. In one embodiment, R^{19a} is selected from the group consisting of substituted and unsubstituted straight-chain C₁₋₂₀-alkyl, substituted and unsubstituted branched-chain C₃₋₂₀-alkyl, and substituted and unsubstituted C₃₋₂₀-cycloalkyl. In another embodiment, R^{19a} is selected from the group consisting of substituted and unsubstituted C₆₋₂₀-aryl, and substituted and unsubstituted C₄₋₂₀-heteroaryl wherein the heteroatoms are independently selected from sulfur, nitrogen and oxygen. R^{19a} may be substituted or unsubstituted branched- or straight-chain alkyl groups such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl or aryl groups such as phenyl, naphthyl or anthracyl. In one embodiment, the alkyl groups may be optionally substituted with one or more (e.g. 1, 2, 3, 4, or 5) substituents each of which may be the same or different such as halide (F, Cl, Br or I), alkoxy groups, e.g. methoxy, ethoxy or propoxy. The aryl group may be optionally substituted with one or more (e.g. 1, 2, 3, 4, or 5) substituents each of which may be the same or different such as halide (F, Cl, Br or I), straight- or branched-chain alkyl (e.g. C₁-C₁₀), alkoxy (e.g. C₁-C₁₀ alkoxy), straight- or branched-chain (dialkyl)amino (e.g. C₁-C₁₀ dialkyl)amino), heterocycloalkyl (e.g. C₃₋₁₀ heterocycloalkyl groups, such as morpholiny/and piperadinyl) or tri(halo)methyl (e.g. F₃C—). Suitable substituted aryl groups include but are not limited to 2, 3- or 4-dimethylaminophenyl, 2, 3- or 4-methylphenyl, 2,3- or 3,5-dimethylphenyl, 2, 3- or 4-methoxyphenyl and 4-methoxy-3,5-dimethylphenyl. Substituted or unsubstituted heteroaryl groups such as pyridyl may also be used. In one embodiment, each R¹⁹ is independently a methyl, phenyl or substituted phenyl group.

In preferred processes of the present invention, the Group 10 transition metal catalyst is selected from

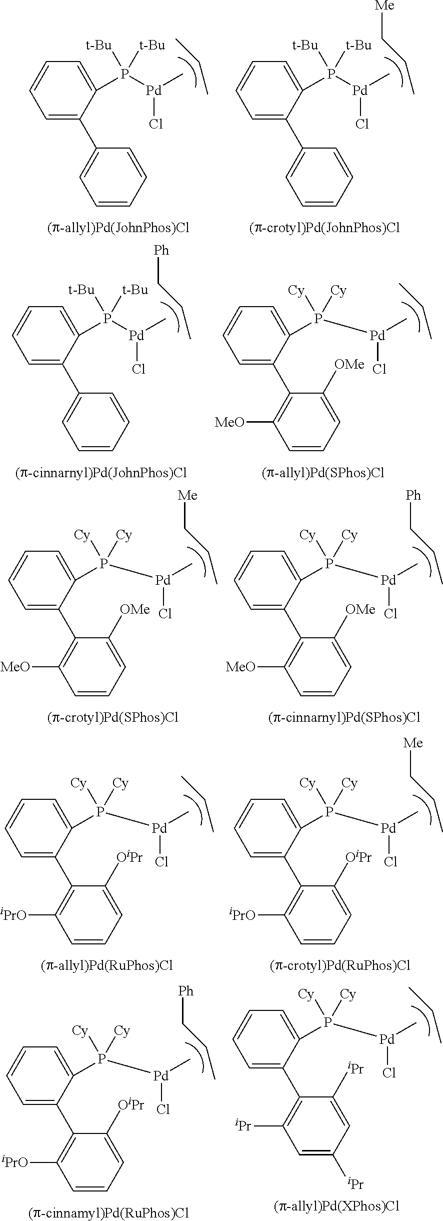

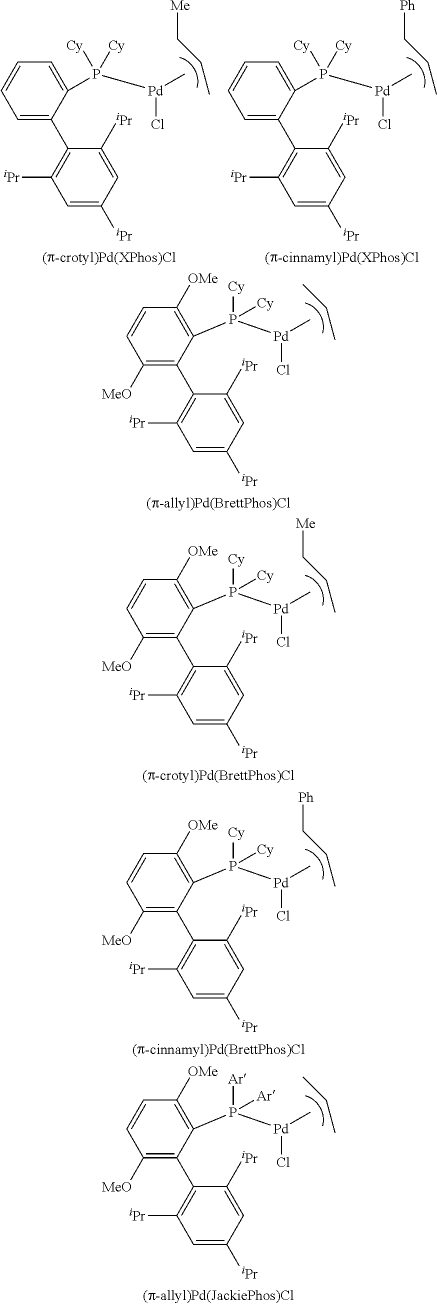

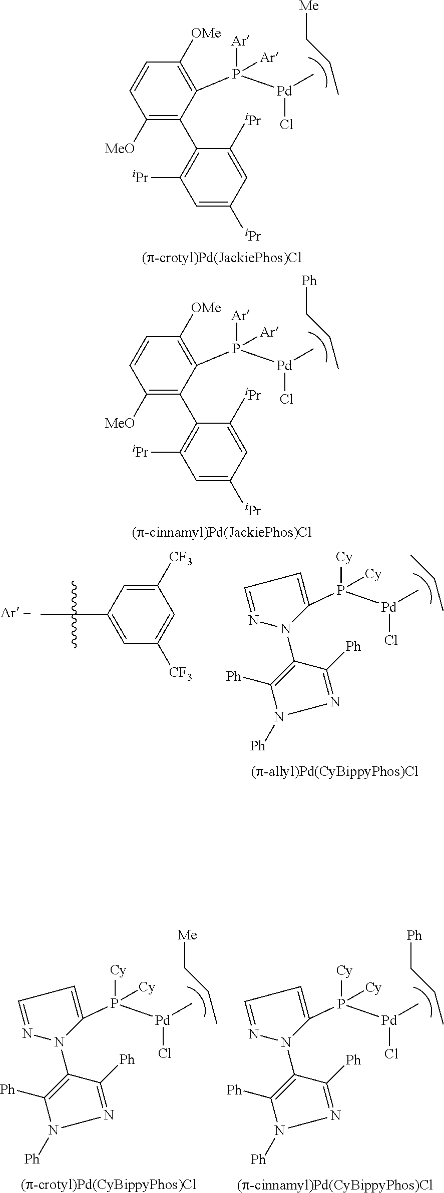

In altemative preferred processes of the present invention, the Group 10 transition metal catalyst is of formula (Cb)

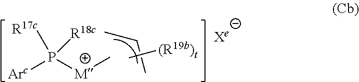

wherein,
M″⊕ is a cationic Group 10 transition metal atom, preferably a cationic palladium atom;
X^{e}\textcircled{−} is a non-coordinated anionic ligand;
Ar^{c} is a substituted or unsubstituted aryl group or a substituted or unsubstituted heteroaryl group;
R^{17c} and R^{18c} are independently organic groups having 1-20 carbon atoms, or R^{17c} and R^{18c} are linked to form a ring structure with P;
R^{19b} is an organic group having 1-20 carbon atoms; and
t is 0, 1, 2, 3, 4 or 5.

In the Group 10 transition metal catalysts of formula (Cb), X^{e}\textcircled{−} is a non-coordinated anionic ligand. By “non-coordinated anion ligand”, we mean the anionic ligand is forced to the outer sphere of the metal centre. The anionic ligand, therefore, is dissociated from the metal centre. This is in contrast to neutral complexes in which the anionic ligand is bound to the metal within the coordination sphere. The anionic ligand can be generally identified as non-coordinating by analysing the X-ray crystal structure of the cationic complex. In one embodiment, X^{e}\textcircled{−} is selected from the group consisting of triflate (i.e. TfO⁻ or CF₃SO₃⁻), tetrafluoroborate (i.e. ⁻BF₄), hexafluoroantimonate (i.e. ⁻SbF₆), hexafluorophosphate (PF₆⁻), [B[3,5-(CF₃)₂C₆H₃]₄]⁻ ([Bar^{F}₄]⁻) and mesylate (MsO⁻ or MeSO₃⁻).

In the Group 10 transition metal catalysts of formula (Cb), R^{17c} and R^{18c} may be the same or different. In one embodiment, R^{17c} and R^{18c} are the same. In another embodiment, R^{17c} and R^{18c} are different. R^{17c} and R^{18c} are selected up to the limitations imposed by stability and the rules of valence. R^{17c} and R^{18c} may be independently selected from the group consisting of substituted and unsubstituted straight-chain C₁₋₂₀-alkyl, substituted and unsubstituted branched-chain C₃₋₂₀-alkyl, substituted and unsubstituted C₃₋₂₀-cycloalkyl, substituted and unsubstituted C₆₋₂₀-aryl, and substituted and unsubstituted C₄₋₂₀-heteroaryl wherein the heteroatoms are independently selected from sulfur, nitrogen and oxygen. R^{17c} and R^{18c} may independently be substituted or unsubstituted branched- or straight-chain alkyl groups such as methyl, ethyl, n-propyl, iso-propyl, n-butyl, iso-butyl, sec-butyl, tert-butyl, pentyl (e.g. n-pentyl or neopentyl), hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In one embodiment, the alkyl groups may be optionally substituted with one or more (e.g. 1, 2, 3, 4, or 5) substituents each of which may be the same or different such as halide (F, Cl, Br or I) or alkoxy groups, e.g. methoxy, ethoxy or propoxy. The aryl group may be optionally substituted with one or more (e.g. 1, 2, 3, 4, or 5) substituents each of which may be the same or different such as halide (F, Cl, Br or I), straight- or branched-chain alkyl (e.g. C₁-C₁₀), alkoxy (e.g. C₁-C₁₀ alkoxy), straight- or branched-chain (dialkyl)amino (e.g. C₁-C₁₀ dialkyl)amino), heterocycloalkyl (e.g. C₃₋₁₀ heterocycloalkyl groups, such as morpholinyl and piperadinyl) or tri(halo)methyl (e.g. F₃C—). Suitable substituted aryl groups include but are not limited to 4-dimethylaminophenyl, 4-methylphenyl, 3,5-dimethylphenyl, 4-methoxyphenyl, 4-methoxy-3,5-dimethylphenyl and 3,5-di(trifluoromethyl)phenyl. Substituted or unsubstituted heteroaryl groups such as pyridyl may also be used. In an alternative embodiment, R^{17c} and R^{18c} are linked to form a ring structure with P, preferably 4- to 7-membered rings. Preferably, R^{17c} and R^{18c} are the same and are tert-butyl, cyclohexyl, adamantyl, phenyl or substituted phenyl groups, such as 3,5-di(trifluoromethyl)phenyl.

In the Group 10 transition metal catalysts of formula (Cb), Ar^{c} is a substituted or unsubstituted aryl group, or a substituted or unsubstituted heteroaryl group. Preferably, Ar^{c} is a substituted or unsubstituted phenyl group, a substituted or unsubstituted biphenyl group, a substituted or unsubstituted napthyl group, a substituted or unsubstituted binapthyl group, a substituted or unsubstituted pyrazolyl group, or a substituted or unsubstituted bipyrazolyl group. More preferably, Ar^{c} is a substituted or unsubstituted biphenyl group, a substituted or unsubstituted binapthyl group, or a substituted or unsubstituted bipyrazolyl group. Even more preferably, Ar^{c} is a substituted or unsubstituted biphenyl group, or a substituted or unsubstituted bipyrazolyl group.

In the Group 10 transition metal catalysts of formula (Cb), the phosphine ligand —P(R^{17c})(R^{18c})Ar^{c} is preferably selected from the group consisting of:

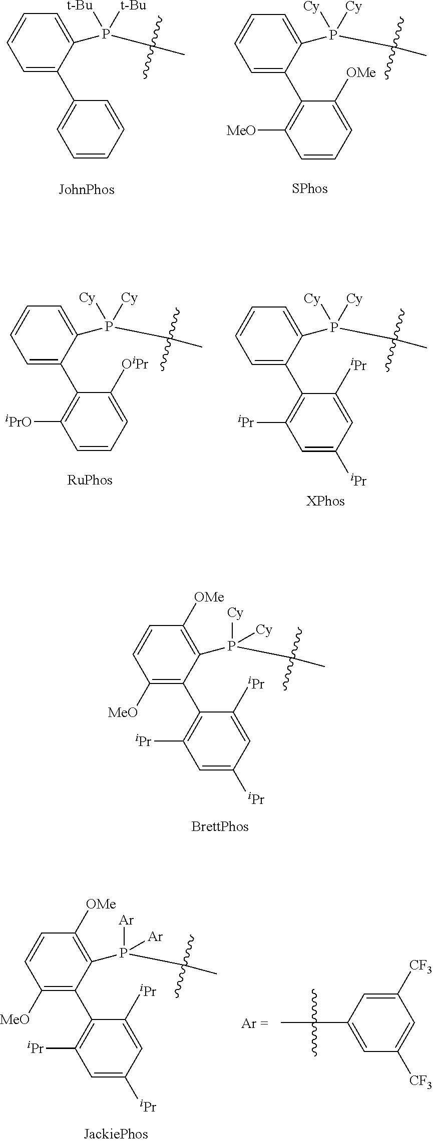

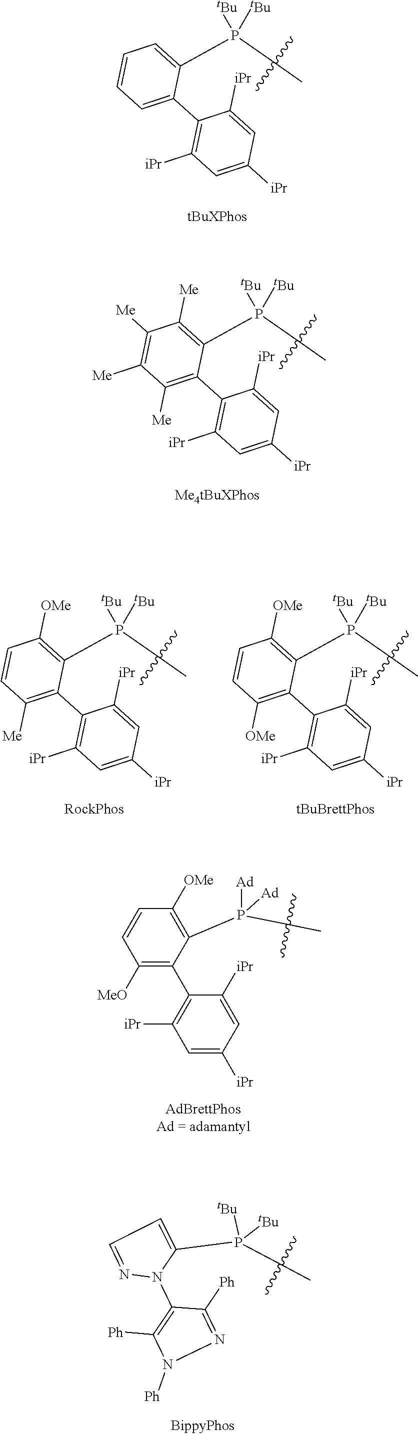

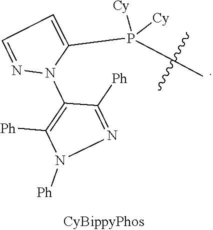

The metal cation in the Group 10 transition metal catalysts of formula (Cb) is coordinated to an optionally substituted allyl group. R^{19b} is an organic group having 1-20 carbon atoms, preferably 1-10 carbon atoms and more preferably 1-8 carbon atoms. R^{19b} is selected up to the limitations imposed by stability and the rules of valence. The number of R^{19b} groups ranges from 0 to 5 i.e. t is 0, 1, 2, 3, 4 or 5. When t is 2, 3, 4 or 5, each of R^{18b} may be the same or different. In certain embodiments, when tis 2, 3, 4, or 5, each R^{19b} is the same. In certain embodiments, t is 0 i.e. the allyl group is unsubstituted. In certain embodiments, t is 1. In certain embodiments, t is 2, wherein each R^{19b} is the same or different.

In the Group 10 transition metal catalysts of formula (Ca), R^{19b} may be selected from the group consisting of substituted and unsubstituted straight-chain alkyl, substituted and unsubstituted branched-chain alkyl, substituted and unsubstituted cycloalkyl, substituted and unsubstituted aryl, and substituted and unsubstituted heteroaryl wherein the heteroatoms are independently selected from sulfur, nitrogen and oxygen. In one embodiment, R^{19b} is selected from the group consisting of substituted and unsubstituted straight-chain alkyl, substituted and unsubstituted branched-chain alkyl, and substituted and unsubstituted cycloalkyl. In another embodiment, R^{19b} is selected from the group consisting of substituted and unsubstituted aryl, and substituted and unsubstituted heteroaryl wherein the heteroatoms are independently selected from sulfur, nitrogen and oxygen. R^{19b} may be substituted or unsubstituted branched- or straight-chain alkyl groups such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl or aryl groups such as phenyl, naphthyl or anthracyl. In one embodiment, the alkyl groups may be optionally substituted with one or more (e.g. 1, 2, 3, 4, or 5) substituents each of which may be the same or different such as halide (F, Cl, Br or I), alkoxy groups, e.g. methoxy, ethoxy or propoxy. The aryl group may be optionally substituted with one or more (e.g. 1, 2, 3, 4, or 5) substituents each of which may be the same or different such as halide (F, Cl, Br or l), straight- or branched-chain alkyl (e.g. C₁-C₁₀), alkoxy (e.g. C₁-C₁₀ alkoxy), straight- or branched-chain (dialkyl)amino (e.g. C₁-C₁₀ dialkyl)amino), heterocycloalkyl (e.g. C₃₋₁₀ heterocycloalkyl groups, such as morpholinyl and piperadinyl) or tri(halo)methyl (e.g. F₃C—). Suitable substituted aryl groups include but are not limited to 2, 3- or 4-dimethylaminophenyl, 2, 3- or 4-methylphenyl, 2,3- or 3,5-dimethylphenyl, 2, 3- or 4-methoxyphenyl and 4-methoxy-3,5-dimethylphenyl. Substituted or unsubstituted heteroaryl groups such as pyridyl may also be used. In one embodiment, each R^{19b} is independently a methyl, phenyl or substituted phenyl group.

In preferred processes of the present invention, the Group 10 transition metal catalyst is selected from:

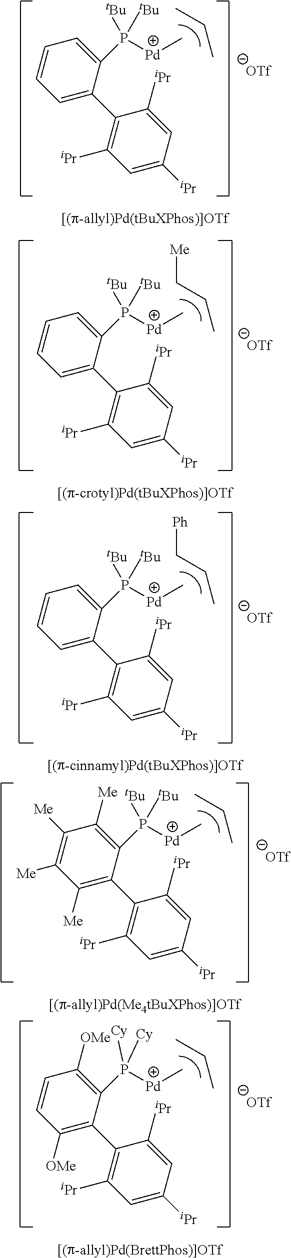

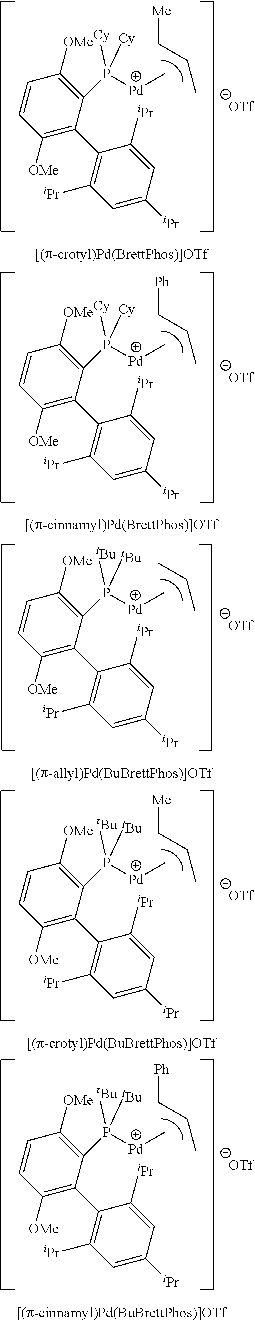

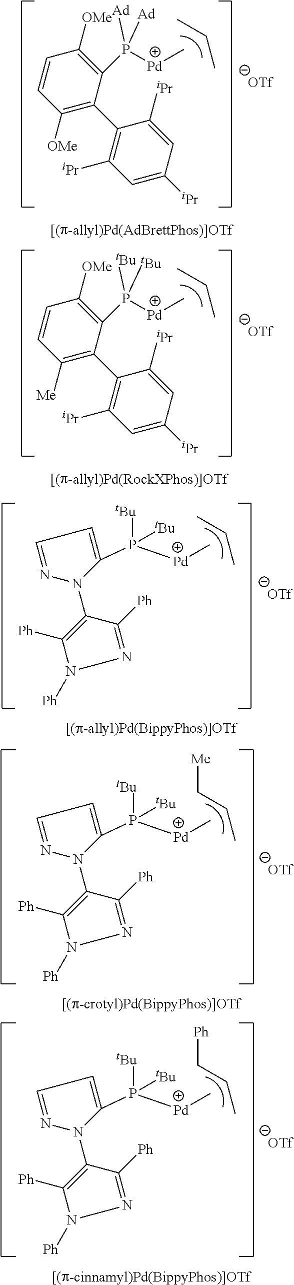

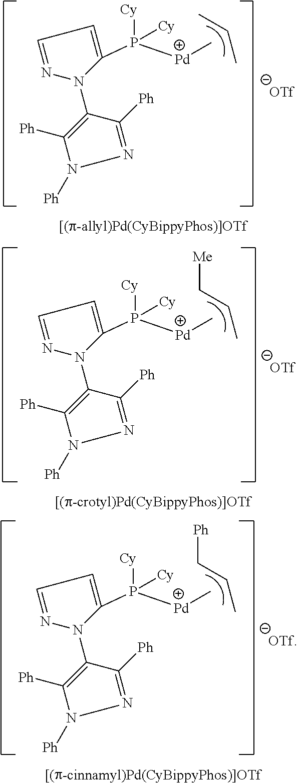

In preferred processes of the present invention, the Group 10 transition metal catalyst is a catalyst of formula (A), (B), (Ca), or (Cb), preferably a catalyst of formula (A). In particularly preferred processes of the present invention, the Group 10 transition metal catalyst is PdCl₂(dippf).

In preferred processes of the present invention, the Group 10 transition metal catalyst is present in an amount of 0.1 to 3.0 mol % based upon the total amount of compound (V), preferably 0.5 to 2.9 mol % based upon the total amount of compound (V), preferably 1.5 to 2.8 mol % based upon the total amount of compound (V), more preferably 2.0 to 2.7 mol % based upon the total amount of compound (V), more preferably 2.25 to 2.6 mol % based upon the total amount of compound (V), more preferably 2.5 to 2.6 mol % based upon the total amount of compound (V) (e.g. 2.5 mol % based upon the total amount of compound (V)).

Without wishing to be bound by theory, it is thought that the use of higher catalyst loadings within these ranges decreases the amount of impurities formed during the coupling reaction (e.g. as a result of deboronation of the 7-azaindole moiety) and therefore allows for easier purification of the reaction mixture. Thus, in particularly preferred processes of the present invention, the Group 10 transition metal catalyst is present in an amount of at least 2.0 mol % based upon the total amount of compound (V), more preferably at least 2.25 mol % based upon the total amount of compound (V), even more preferably at least 2.5 mol % based upon the total amount of compound (V).

The base must be selected to ensure that it is strong enough to allow the coupling reaction to progress, but not so strong that the carbonate compound of formula (V) will degrade in its presence. The choice of base may depend upon the particular reaction conditions employed. The skilled person is able to select a suitable base.

In preferred processes of the present invention, the base used in the reaction between the compound of formula (V) and the compound of formula (IX) is selected from a metal alkoxide, a metal carbonate, a metal phosphate, a metal hydroxide, and an amine base. Preferably, the base is selected from an alkali metal alkoxide, an alkali metal carbonate, an alkali metal phosphate, an alkali metal hydroxide, and an amine base. More preferably, the base is selected from LiO^{t}Bu, NaO^{t}Bu, KO^{t}Bu, Na₂CO₃, K₂CO₃, Na₃PO₄, K₃PO₄, NaOH, KOH, Et₃N, Et₂ⁱPrN, DMAP, DABCO, or DBU. Most preferably, the base is K₂CO₃ or KO^{t}Bu.

In some preferred processes of the present invention, the base is an alkoxide base (e.g. a metal alkoxide). Thus, in preferred processes of the present invention, the base used in the reaction between the compound of formula (V) and the compound of formula (IX) is a metal alkoxide, preferably an alkali metal alkoxide. More preferably, the base is selected from LiO^{t}Bu, NaO^{t}Bu, and KO^{t}Bu. Most preferably, the base is KO^{t}Bu.

In alternative preferred processes of the present invention, the base is not an alkoxide base (e.g. a metal alkoxide). Thus, in preferred processes of the present invention, the base used in the reaction between the compound of formula (V) and the compound of formula (IX) is selected from a metal carbonate, a metal phosphate, a metal hydroxide, and an amine base. Preferably, the base is selected from an alkali metal carbonate, an alkali metal phosphate, an alkali metal hydroxide, and an amine base. More preferably, the base is selected from Na₂CO₃, K₂CO₃, Na₃PO₄, K₃PO₄, NaOH, KOH, Et₃N, Et₂ⁱPrN, DMAP, DABCO, or DBU. Most preferably, the base is K₂CO₃.

In preferred processes of the present invention, the base is a metal carbonate, preferably an alkali metal carbonate. In particularly preferred processes of the present invention, the base is selected from Na₂CO₃ and K₂CO₃. Most preferably, the base is K₂CO₃.

In preferred processes of the present invention, the base is a metal phosphate, preferably an alkali metal phosphate. In particularly preferred processes of the present invention, the base is selected from Na₃PO₄ and K₃PO₄. Most preferably, the base is K₃PO₄.

In preferred processes of the present invention, the base is a metal hydroxide, preferably an alkali metal hydroxide. In particularly preferred processes of the present invention, the base is selected from NaOH and KOH. Most preferably, the base is KOH.

In preferred processes of the present invention, the base is an amine base. Preferably, the amine base is compound having a formula selected from R′—NH₂, R′₂NH, and R′₃N, wherein R′ are independently alkyl, aryl or heteroaryl groups, or the amine base is a cyclic amine base. In particularly preferred processes of the present invention, the base is selected from Et₃N, Et₂ⁱPrN, DMAP, DABCO, or DBU. Most preferably, the base is Et₃N.

In preferred processes of the present invention, the base used in the reaction between the compound of formula (V) and the compound of formula (IX) is present in an amount of 50 to 300 mol % based upon the total amount of the compound of formula (V), more preferably 100 to 250 mol % based upon the total amount of the compound of formula (V), more preferably 150 to 200 mol % based upon the total amount of the compound of formula (V).

In preferred processes of the present invention, water is present in an amount of 2000 to 4000 mol % based upon the total amount of the compound of formula (V), more preferably 1500 to 3500 mol % based upon the total amount of the compound of formula (V) (e.g. 3000 mol % based upon the total amount of the compound of formula (V)).

In preferred processes of the present invention, R² in formula (V) is a substituted or unsubstituted C₁₋₁₀ straight-chain or C₃₋₁₀ branched-chain alkyl group, preferably a substituted or unsubstituted C₁₋₅ straight-chain or C₃₋₅ branched-chain alkyl group. More preferably, R² in formula (V) is methyl, ethyl, iso-propyl or tert-butyl. Most preferably, R² in formula (V) is methyl.

In preferred processes of the present invention, Z in formula (IX) is —CO₂R³. Preferably, R³ is a substituted or unsubstituted C₁₋₁₀ straight-chain or C₃₋₁₀ branched-chain alkyl group or a substituted or unsubstituted C₆₋₁₀ aryl group. Particularly preferred C₁₋₁₀-alkyl groups include methyl, ethyl, n-propyl, iso-propyl, n-butyl, sec-butyl, tert-butyl, pentyl, and hexyl, more preferably methyl, ethyl, iso-propyl, tert-butyl, even more preferably tert-butyl. Preferred C₆₋₁₀-aryl groups include phenyl, tolyl, xylyl, and methoxyphenyl, more preferably phenyl.

In preferred processes of the present invention, Z in formula (IX) is —SO₂R″. Preferably, R″ is a substituted or unsubstituted C₁₋₁₀ straight-chain or C₃₋₁₀ branched-chain alkyl group or a substituted or unsubstituted C₆₋₁₀ aryl group. Particularly preferred C₁₋₁₀-alkyl groups include methyl, trifluoromethyl, ethyl, n-propyl, iso-propyl, n-butyl, sec-butyl, tert-butyl, pentyl, and hexyl, more preferably methyl, ethyl, iso-propyl, and tert-butyl, even more preferably methyl and ethyl. Preferred C₆₋₁₀-aryl groups include phenyl, tolyl, xylyl, methoxyphenyl, bromophenyl, and nitrophenyl, more preferably tolyl.

In preferred processes of the present invention, R⁴ and R⁵ in formula (IX) are, independently, H or a substituted or unsubstituted C₁₋₂₀ straight-chain or C₃₋₂₀ branched-chain alkyl group; or, together with the atoms to which they are attached, R⁴ and R⁵ form a ring.

In preferred processes of the present invention, R⁴ and R⁵ in formula (IX) are, independently, H or a substituted or unsubstituted C₁₋₁₀ straight-chain or C₃₋₁₀ branched-chain alkyl group, more preferably H or a substituted or unsubstituted C₁₋₅ straight-chain or C₃₋₅ branched-chain alkyl group, even more preferably H.

In alternative preferred processes of the present invention, together with the atoms to which they are attached, R⁴ and R⁵ in formula (IX) form a ring. More preferably, R⁴ and R⁵ form a ring which is —Bpin or —Bcat. Even more preferably, R⁴ and R⁵ form a ring which is —Bpin.

In preferred processes of the present invention, the reacting of the compound of formula (V) with the compound of formula (IX) is conducted at a temperature in the range 5 to 100° C., more preferably 10 to 90° C., even more preferably 20 to 80° C., and even more preferably 50 to 70° C.

In preferred processes of the present invention, the reacting of the compound of formula (V) with the compound of formula (IX) is conducted for a duration of 4 to 10 hours, more preferably 5 to 9 hours, even more preferably 6 to 8 hours.

In preferred processes of the present invention, the reacting of the compound of formula (V) with the compound of formula (IX) is carried out in a solvent. The solvent may be an alcohol, an ether (e.g. tetrahydrofuran, methyltetrahydrofuran), an aromatic solvent (e.g. toluene), an alkyl solvent (e.g. heptane), a dialkylcarbonate (e.g. dimethylcarbonate), or a mixture thereof. Preferably, the solvent is an alcohol or mixture of alcohols. More preferably, the solvent is iso-propanol and/or tert-amyl alcohol. Alternatively, the solvent is a dialkylcarbonate, preferably dimethylcarbonate, diethyl carbonate, di-iso-propyl carbonate, or di-tert-butylcarbonate, most preferably dimethylcarbonate or di-tert-butylcarbonate.

In preferred processes of the present invention, the solvent is an alcohol and is present in an amount of 1000 to 5000 mol % based upon the total amount of said compound of formula (V), preferably 1500 to 4500 mol % based upon the total amount of said compound of formula (V), more preferably 2000 to 4000 mol % based upon the total amount of said compound of formula (V).

In preferred processes of the present invention, the reaction of the compound of formula (V) with the compound of formula (IX) takes place in the presence of a Lewis acid. Preferably, the Lewis acid is selected from a metal halide, a metal carbonate, or a metal phosphate, such as lithium chloride, lithium bromide, lithium iodide, lithium carbonate or lithium phosphate. More preferably, the Lewis acid is a metal halide. Even more preferably, the Lewis acid is an alkali metal halide. Even more preferably, the Lewis acid is selected from lithium chloride, lithium bromide and lithium iodide. Most preferably, the Lewis acid is selected from lithium bromide and lithium iodide. Advantageously, lithium bromide and lithium iodide have increased solubility in organic solvents. Without wishing to be bound by theory, it is thought that when the reaction of the compound of formula (V) with the compound of formula (IX) takes place in the presence of a Lewis acid, the Lewis acid can help to prevent amine groups in the starting materials from coordinating to the Group 10 transition metal catalyst and poisoning it. This means that a lower catalyst loading is required for the coupling reaction.

In preferred processes of the present invention, the Lewis acid is present in an amount of at least 100 mol % based upon the total amount of the compound of formula (V). For example, it may be preferred that the Lewis acid is present in at least 110 mol %, at least 120 mol %, at least 130 mol %, or at least 150 mol % based upon the total amount of the compound of formula (V). There is no particular upper limit for the amount of the Lewis acid which may be present. Typically, the Lewis acid is present in at most 400 mol % based upon the total amount of the compound of formula (V). For example it may be preferred that the Lewis acid is present in at most 350 mol %, at most 300 mol %, at most 250 mol %, or at most 200 mol % based upon the total amount of the compound of formula (V). Preferably, the Lewis acid is present in the range of from 100 to 400 mol % based upon the total amount of the compound of formula (V), more preferably from 110 to 350 mol %, more preferably 120 to 300 mol %, more preferably 130 to 250 mol %, even more preferably 150 to 200 mol % based upon the total amount of the compound of formula (V).

In preferred processes of the present invention, the compound of formula (V) is prepared in situ in the presence of the compound of formula (IX), the Group 10 transition metal catalyst, the base and water by reacting a compound of formula (IV), or a salt thereof,

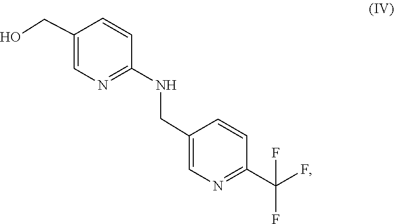 [SMILES: OCc1ccc(NCc2ccc(C(F)(F)F)nc2)nc1]

with a dialkyl carbonate of formula R²O(CO)OR², wherein each R² is a substituted or unsubstituted C₁₋₂₀ straight-chain or C₃₋₂₀ branched-chain alkyl group.

The R² groups in the dialkyl carbonate of formula R²O(CO)OR² may be the same or different. Preferably, the R² groups in the dialkyl carbonate of formula R²O(CO)OR² are the same.

In preferred processes of the present invention, the dialkyl carbonate is selected from dimethylcarbonate, diethyl carbonate, di-iso-propyl carbonate, and di-tert-butylcarbonate. Most preferably, the dialkyl carbonate is dimethylcarbonate.

In preferred processes of the present invention, the dialkyl carbonate is present in an amount of 1000 to 5000 mol % based upon the total amount of said compound of formula (IV) or salt thereof, preferably 1500 to 4500 mol % based upon the total amount of said compound of formula (IV) or salt thereof, more preferably 2000 to 4000 mol % based upon the total amount of said compound of formula (IV) or salt thereof.

In preferred processes of the present invention, the base used in the reaction between the compound of formula (V) and the compound of formula (IX) wherein the compound of formula (V) is prepared in situ is selected from a metal alkoxide, a metal carbonate, a metal phosphate, a metal hydroxide, and an amine base. Preferably, the base is selected from an alkali metal alkoxide, an alkali metal carbonate, an alkali metal phosphate, an alkali metal hydroxide, and an amine base. More preferably, the base is selected from LiO^{t}Bu, NaO^{t}Bu, KO^{t}Bu, Na₂CO₃, K₂CO₃, Na₃PO₄, K₃PO₄, NaOH, KOH, Et₃N, Et₂ⁱPrN, DMAP, DABCO, or DBU. Most preferably, the base is K₂CO₃ or KO^{t}Bu.

In particularly preferred processes of the present invention, the base used in the reaction between the compound of formula (V) and the compound of formula (IX) wherein the compound of formula (V) is prepared in situ is a metal alkoxide, preferably an alkali metal alkoxide. More preferably, the base is selected from LiO^{t}Bu, NaO^{t}Bu, and KO^{t}Bu. Most preferably, the base is KO^{t}Bu.

In preferred processes of the present invention, the reacting of the compound of formula (V) with the compound of formula (IX) wherein the compound of formula (V) is prepared in situ is carried out in a solvent. The solvent may be an alcohol, an ether (e.g. tetrahydrofuran, methyltetrahydrofuran), an aromatic solvent (e.g. toluene), an alkyl solvent (e.g. heptane), or a mixture thereof. As will be clearly understood by a skilled person, the dialkylcarbonate of formula R²O(CO)OR² present in the reaction mixture will also function as a solvent during the reaction.

Alternatively, the compound of formula (V) can be prepared in a separate step. Thus, in preferred processes of the present invention, the compound of formula (V) is prepared by reacting a compound of formula (IV), or a salt thereof,

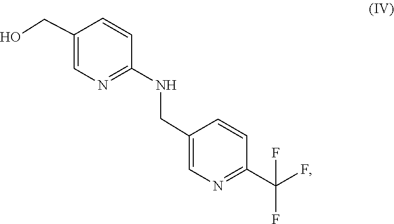 [SMILES: OCc1ccc(NCc2ccc(C(F)(F)F)nc2)nc1]

with a dialkyl carbonate of formula R²O(CO)OR², wherein each R² is a substituted or unsubstituted C₁₋₂₀ straight-chain or C₃₋₂₀ branched-chain alkyl group, in the presence of a base.

In preferred processes of the present invention, the base used in the separate step to produce a compound of formula (V) from a compound of formula (IV), or a salt thereof, is a metal alkoxide. The metal alkoxide is preferably a metal methoxide, a metal ethoxide, a metal iso-propoxide, or a metal tert-butoxide.

In preferred processes of the present invention, the base is an alkali metal alkoxide. The alkali metal alkoxide is preferably an alkali metal methoxide, an alkali metal ethoxide, an alkali metal iso-propoxide, or an alkali metal tert-butoxide. The alkali metal alkoxide is more preferably an alkali metal methoxide or an alkali metal ethoxide, preferably an alkali metal methoxide.

Preferred metal alkoxides include sodium methoxide, sodium ethoxide, potassium methoxide, or potassium ethoxide, preferably sodium methoxide.

In preferred processes of the present invention, the base used in the separate step to produce a compound of formula (V) from a compound of formula (IV), or a salt thereof, is present in an amount of 1 to 10 mol % based upon the total amount of said compound of formula (IV) or salt thereof, preferably 2 to 9 mol % based upon the total amount of said compound of formula (IV) or salt thereof, more preferably 3 to 8 mol % based upon the total amount of said compound of formula (IV) or salt thereof (e.g. 5 mol % based upon the total amount of said compound of formula (IV) or salt thereof).

In preferred processes of the present invention, the dialkyl carbonate is present in an amount of 500 to 2000 mol % based upon the total amount of said compound of formula (IV) or salt thereof, preferably 750 to 1000 mol % based upon the total amount of said compound of formula (IV) or salt thereof.

In preferred processes of the present invention, the base used in the reaction between the compound of formula (V) and the compound of formula (IX) wherein the compound of formula (V) is prepared in a separate step is selected from a metal carbonate, a metal phosphate, a metal hydroxide, and an amine base. Preferably, the base is selected from an alkali metal carbonate, an alkali metal phosphate, an alkali metal hydroxide, and an amine base. More preferably, the base is selected from Na₂CO₃, K₂CO₃, Na₃PO₄, K₃PO₄, NaOH, KOH, Et₃N, Et₂ⁱPrN, DMAP, DABCO, or DBU. Most preferably, the base is K₂CO₃.

In preferred processes of the present invention, the reacting of the compound of formula (V) with the compound of formula (IX) wherein the compound of formula (V) is prepared in a separate step is carried out in a solvent. The solvent may be an alcohol, an ether (e.g. tetrahydrofuran, methyltetrahydrofuran), an aromatic solvent (e.g. toluene), an alkyl solvent (e.g. heptane), a dialkylcarbonate (e.g. dimethylcarbonate), or a mixture thereof.

Preferably, the solvent is an alcohol or mixture of alcohols. More preferably, the solvent is iso-propanol and/or tert-amyl alcohol.

Alternatively, the solvent is a dialkylcarbonate, preferably dimethylcarbonate, diethyl carbonate, di-iso-propylcarbonate or di-tert-butylcarbonate, most preferably dimethylcarbonate or di-tert-butylcarbonate. Advantageously, when the solvent comprises a dialkylcarbonate this can help to reform the carbonate in the instance it decomposes to the corresponding alcohol under the reaction conditions, and therefore increase reaction yield.

In preferred processes of the present invention, the compound of formula (IV), or a salt thereof, is reacted with the dialkyl carbonate at a temperature in the range 10 to 150° C., preferably 30 to 140° C., more preferably 50 to 130° C., more preferably 65 to 120° C.

In preferred processes of the present invention, the compound of formula (IV), or a salt thereof, is reacted with the dialkyl carbonate for a duration of 1 to 6 hours, preferably 2 to 5 hours, more preferably 3 to 4 hours.

In preferred processes of the present invention, the compound of formula (IV), or a salt thereof, is reacted with a dialkyl carbonate of formula R²O(CO)OR², wherein each R² is a substituted or unsubstituted C₁₋₂₀ straight-chain or C₃₋₂₀ branched-chain alkyl group. The R² groups in the dialkyl carbonate of formula R²O(CO)OR² may be the same or different. Preferably, the R² groups in the dialkyl carbonate of formula R²O(CO)OR² are the same.

In preferred processes of the present invention, the dialkyl carbonate is selected from dimethylcarbonate, diethyl carbonate, di-iso-propyl carbonate, and di-tert-butylcarbonate. Most preferably, the dialkyl carbonate is dimethylcarbonate.

As would be understood by a skilled person, the separate step to produce a compound of formula (V) from a compound of formula (IV), or a salt thereof, can be conducted using a Dean-Stark apparatus. In such an experimental set up, the byproduct collected in the trap (e.g. methanol in the instance the reagent is dimethyl carbonate) can be removed at regular intervals, or the trap can be filled with molecular sieves to avoid the need to drain the trap. During the reaction, additional reagent (e.g. dimethyl carbonate) can be added to the reaction vessel at regular intervals.

In preferred processes of the present invention, the compound of formula (IV) is prepared by reducing a compound of formula (III)

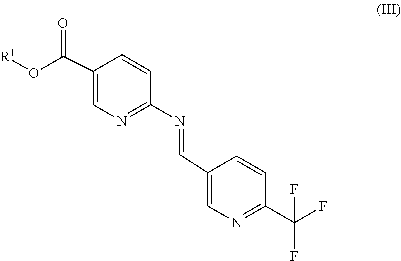

wherein R¹ is a substituted or unsubstituted C₁₋₂₀ straight-chain or C₃₋₂₀ branched-chain alkyl group or a substituted or unsubstituted C₆₋₂₀ aryl group;
with hydrogen in the presence of a Group 8 transition metal catalyst and a base. Such a step increases the efficiency of the overall process because it allows for both the ester moiety and the imine moiety present in the compound of formula (III) to be reduced in a single step.

In preferred processes of the present invention, the compound of formula (III) is reduced in the presence of at least one solvent.

Preferably, the at least one solvent is selected from an alcohol, benzene, toluene, o-xylene, m-xylene, p-xylene, THF and Me-THF. More preferably, the at least one solvent is selected from methanol, ethanol, iso-propanol, tert-butanol, benzene, toluene, o-xylene, m-xylene, p-xylene, THF and Me-THF. Most preferably, the at least one solvent is selected from ethanol, iso-propanol, tert-butanol, o-xylene, m-xylene, p-xylene, and toluene. Most preferably, the at least one solvent is ethanol. Advantageously, it has been found that the use of a single alcohol solvent (e.g. ethanol) results in the formation of less impurities during the reduction reaction, thereby making purification of the reaction mixture easier and the process more amenable to scale up.

In preferred processes of the present invention, the at least one solvent is a single alcohol (e.g. ethanol) which is present in an amount of 1500 to 2500 mol % based upon the total amount of compound (III) (e.g. 1500 mol % based upon the total amount of compound (III)), preferably 1600 to 2000 mol % based upon the total amount of compound (III), more preferably 1700 to 1800 mol % based upon the total amount of compound (III).

In particularly preferred processes of the present invention, the compound of formula (III) is reduced in the presence of a first solvent and a second solvent.

In preferred processes of the present invention, the first solvent is selected from benzene, toluene, o-xylene, m-xylene, p-xylene, THF and Me-THF. More preferably, the first solvent is toluene, o-xylene, m-xylene, or p-xylene. Most preferably, the first solvent is toluene.

In preferred processes of the present invention, the second solvent is an alcohol, preferably ethanol, iso-propanol, or tert-butanol, more preferably iso-propanol or ethanol. Most preferably, the second solvent is ethanol.

In particularly preferred processes of the present invention, the first solvent is toluene and the second solvent is an alcohol, preferably iso-propanol or ethanol. Most preferably, the first solvent is toluene and the second solvent is ethanol.

In preferred processes of the present invention, the first solvent (e.g. toluene) is present in an amount of 710 to 1200 mol % based upon the total amount of compound (III), preferably 800 to 1100 mol % based upon the total amount of compound (III), more preferably 900 to 1000 mol % based upon the total amount of compound (III) (e.g. 900 mol % based upon the total amount of compound (III)).

In preferred processes of the present invention, the second solvent is an alcohol (e.g. ethanol) which is present in an amount of 200 to 400 mol % based upon the total amount of compound (III), preferably 250 to 350 mol % based upon the total amount of compound (III), more preferably 275 to 325 mol % based upon the total amount of compound (III) (e.g. 300 mol % based upon the total amount of compound (III)).

The processes of the present invention employ a Group 8 transition metal catalyst. Preferably, the Group 8 transition metal catalyst is an iron catalyst, a ruthenium catalyst, or an osmium catalyst. More preferably, the Group 8 transition metal catalyst is a ruthenium catalyst or an osmium catalyst. Most preferably, the Group 8 transition metal catalyst is a ruthenium catalyst.

In preferred processes of the present invention, the Group 8 transition metal catalyst comprises a tridentate ligand having a formula (D)

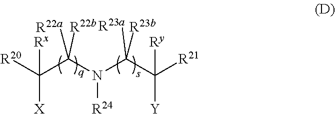

wherein:
X is selected from —SR^{d}, —OR^{d}, —CR^{d}, —NR^{d}R^{e}, —PR^{d}R^{e}, —P(═O)R^{d}R^{e}, —OPR^{d}R^{e}, and —NHPR^{d}R^{e};
R²⁰ and R^{x} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl, or R²⁰ and one of R^{22a} and R^{22b} or R^{x} and one of R^{22a} and R^{22b} together with the atoms to which they are bound, form a ring;
or X is a heteroatom and when taken together with R²⁰ it forms an optionally substituted heterocycle when R^{x} is absent;
Y is selected from —SR^{d}, —OR^{d}, —CR^{d}, —NR^{d}R^{e}, —PR^{d}R^{e}, —P(═O)R^{d}R^{e}, —OPR^{d}R^{e}, and —NHPR^{d}R^{e};
R²¹ and R^{y} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl, or R²¹ and one of R^{23a} and R^{23b} or R^{y} and one of R^{23a} and R^{23b} together with the atoms to which they are bound, form a ring;
or Y is a heteroatom and when taken together with R²¹ it forms an optionally substituted heterocycle when R^{y} is absent;
R^{22a}, R^{22b}, R^{23a}, and R^{23b} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl, or
R^{22a} and one of R^{23a} and R^{23b} or R^{22b} and one of R^{23a} and R^{23b} together with the atoms to which they are bound, form a heterocycle;
R²⁴ is selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
each q and s is independently 1 or 2; and
R^{d} and R^{e}, if present, are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; or when X and/or Y is —NR^{d}R^{e}, —PR^{d}R^{e}, —OPR^{d}R^{e}, or —NHPR^{d}R^{e}, R^{d} and R^{e} together with the heteroatom to which they are attached form a heterocycle.

In tridentate ligands of formula (D), X is preferably selected from —SR^{d}, —CR^{d}, —NR^{d}R^{e}, —PR^{d}R^{e}, and —NHPR^{d}R^{e}. More preferably, X is selected from —SR^{d}, —PR^{d}R^{e}, and —NHPR^{d}R^{e}. Even more preferably, X is selected from —SR^{d} and —PR^{d}R^{e}. Most preferably, X is —SR^{d} (e.g. —SEt).

In tridentate ligands of formula (D), R²⁰ and R^{x} are each independently preferably selected from hydrogen, substituted or unsubstituted C₁₋₂₀-alkyl, substituted or unsubstituted C₁₋₂₀-heteroalkyl and substituted or unsubstituted C₃₋₂₀-cycloalkyl. More preferably, R²⁰ and R^{x} are each independently selected from hydrogen and substituted or unsubstituted C₁₋₂₀-alkyl. Even more preferably, R²⁰ and R^{x} are each hydrogen.

In alternative preferred tridentate ligands of formula (D), X is a heteroatom and when taken together with R²⁰ it forms an optionally substituted heterocycle when R^{x} is absent. More preferably, X is a heteroatom and when taken together with R²⁰ it forms an optionally substituted heteroaromatic ring when R^{x} is absent. More preferably, the optionally substituted heteroaromatic ring is an optionally substituted nitrogen-containing heteroaromatic ring. Even more preferably, the optionally substituted nitrogen-containing heteroaromatic ring is selected from pyridinyl, pyrrolyl, imidazolyl, pyrazolyl, thiazolyl. isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl. Even more preferably, the optionally substituted nitrogen-containing heteroaromatic ring is selected from pyridinyl, pyrrolyl, imidazolyl, pyrazolyl, triazolyl, tetrazolyl, and pyrimidyl. Most preferably, the optionally substituted nitrogen-containing heteroaromatic ring is pyridinyl.

In tridentate ligands of formula (D), Y is preferably selected from —SR^{d}, —CR^{d}, —NR^{d}R^{e}, —PR^{d}R^{e}, and —NHPR^{d}R^{e}. More preferably, Y is selected from —SR^{d}, —PR^{d}R^{e}, and —NHPR^{d}R^{e}. Even more preferably, Y is selected from —SR^{d} and —PR^{d}R^{e}. Most preferably, Y is —SR^{d} (e.g. —SEt).

In tridentate ligands of formula (D), R²¹ and R^{y} are each independently preferably selected from hydrogen, substituted or unsubstituted C₁₋₂₀-alkyl, substituted or unsubstituted C₁₋₂₀-heteroalkyl and substituted or unsubstituted C₃₋₂₀-cycloalkyl. More preferably, R²¹ and R^{y} are each independently selected from hydrogen and substituted or unsubstituted C₁₋₂₀-alkyl. Even more preferably, R²¹ and R^{y} are each hydrogen.

In alternative preferred tridentate ligands of formula (D), Y is a heteroatom and when taken together with R²¹ it forms an optionally substituted heterocycle when R^{y} is absent. More preferably, Y is a heteroatom and when taken together with R²¹ it forms an optionally substituted heteroaromatic ring when R^{y} is absent. More preferably, the optionally substituted heteroaromatic ring is an optionally substituted nitrogen-containing heteroaromatic ring. Even more preferably, the optionally substituted nitrogen-containing heteroaromatic ring is selected from pyridinyl, pyrrolyl, imidazolyl, pyrazolyl, thiazolyl, isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl. Even more preferably, the optionally substituted nitrogen-containing heteroaromatic ring is selected from pyridinyl, pyrrolyl, imidazolyl, pyrazolyl, triazolyl, tetrazolyl, and pyrimidyl. Most preferably, the optionally substituted nitrogen-containing heteroaromatic ring is pyridinyl.

In tridentate ligands of formula (D), R^{22a}, R^{22b}, R^{23a}, and R^{23b} are each independently preferably selected from hydrogen, substituted or unsubstituted C₁₋₂₀-alkyl, substituted or unsubstituted C₁₋₂₀-heteroalkyl and substituted or unsubstituted C₃₋₂₀-cycloalkyl. More preferably, R^{22a}, R^{22b}, R^{23a}, and R^{23b} are each independently selected from hydrogen and substituted or unsubstituted C₁₋₂₀-alkyl. Even more preferably, R^{22a}, R^{22b}, R^{23a}, and R^{23b} are each hydrogen.

In alternative preferred tridentate ligands of formula (D), R^{22a} and one of R^{23a} and R^{23b} or R^{22b} and one of R^{23a} and R^{23b} together with the atoms to which they are bound, form a heterocycle. Preferably, the heterocycle is a six-membered ring heterocycle.

In tridentate ligands of formula (D), R²⁴ is preferably selected from hydrogen, substituted or unsubstituted C₁₋₂₀-alkyl, substituted or unsubstituted C₁₋₂₀-heteroalkyl and substituted or unsubstituted C₃₋₂₀-cycloalkyl. More preferably, R²⁴ is selected from hydrogen and substituted or unsubstituted C₁₋₂₀-alkyl. Even more preferably, R²⁴ is hydrogen.

In tridentate ligands of formula (D), each q and s is preferably 1.

In tridentate ligands of formula (D), R^{d} and R^{e}, if present, are each independently preferably selected from hydrogen, substituted or unsubstituted C₁₋₂₀-alkyl, substituted or unsubstituted C₁₋₂₀-heteroalkyl, substituted or unsubstituted C₃₋₂₀-cycloalkyl, substituted or unsubstituted C₆₋₂₀-aryl, and substituted or unsubstituted C₄₋₂₀-heteroaryl. More preferably, R^{d} and R^{e}, if present, are each independently selected from hydrogen, substituted or unsubstituted C₁₋₂₀-alkyl (e.g. C₁₋₁₀-alkyl) and substituted or unsubstituted C₆₋₂₀-aryl. Particularly preferred C₁₋₂₀-alkyl groups include methyl, ethyl, n-propyl, iso-propyl, n-butyl, sec-butyl, tert-butyl, pentyl, and hexyl, more preferably methyl, ethyl, iso-propyl, tert-butyl, even more preferably ethyl. Preferred C₆₋₂₀-aryl groups include phenyl, tolyl, xylyl, and methoxyphenyl, more preferably phenyl.

In alternative preferred tridentate ligands of formula (D), when X and/or Y is —NR^{d}R^{e}, —PR^{d}R^{e}, —OPR^{d}R^{e}, or —NHPR^{d}R^{e}, R^{d} and R^{e} together with the heteroatom to which they are attached form a heterocycle.

In preferred processes of the present invention, the Group 8 transition metal catalyst comprises a tridentate ligand having a formula (D)
wherein:
X is selected from —SR^{d}, —CR^{e}, —NR^{d}R^{e}, —PR^{d}R^{e}, and —NHPR^{d}R^{e};
R²⁰ and R^{x} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
or X is a heteroatom and when taken together with R²⁰ it forms an optionally substituted heteroaromatic ring when R^{x} is absent, wherein the heteroaromatic ring is a nitrogen-containing heteroaromatic ring;
Y is selected from —SR^{d}, —CR^{d}, —NR^{d}R^{e}, —PR^{d}R^{e}, and —NHPR^{d}R^{e};
R²¹ and R^{y} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
or Y is a heteroatom and when taken together with R²¹ it forms an optionally substituted heteroaromatic ring when R^{y} is absent, wherein the heteroaromatic ring is a nitrogen-containing heteroaromatic ring;
R^{22a}, R^{22b}, R^{23a}, R^{23b} and R²⁴ are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; each q and s is independently 1 or 2; and
R^{d} and R^{e}, if present, are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; or when X and/or Y is —NR^{d}R^{e}, —PR^{d}R^{e}or —NHPR^{d}R^{e}, R^{d} and R^{e} together with the heteroatom to which they are attached form a heterocycle.

In preferred processes of the present invention, the Group 8 transition metal catalyst comprises a tridentate ligand having a formula (D)
wherein:
X is selected from —SR^{d}, —PR^{d}R^{e}, and —NHPR^{d}R^{e};
R²⁰ and R^{x} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
or X is a heteroatom and when taken together with R²⁰ it forms an optionally substituted heteroaromatic ring when R^{x} is absent, wherein the heteroaromatic ring is a nitrogen-containing heteroaromatic ring selected from pyridinyl, pyrrolyl, imidazolyl, pyrazolyl, thiazolyl, isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl;
Y is selected from —SR^{d}, —PR^{d}R^{e}, and —NHPR^{d}R^{e};
R²¹ and R^{y} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
or Y is a heteroatom and when taken together with R²¹ it forms an optionally substituted heteroaromatic ring when R^{y} is absent, wherein the heteroaromatic ring is a nitrogen-containing heteroaromatic ring selected from pyridinyl, pyrrolyl, imidazolyl, pyrazolyl, thiazolyl, isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl;
R^{22a}, R^{22b}, R^{23a}, R^{23b} and R²⁴ are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
each q and s is independently 1 or 2; and
R^{d} and R^{e}, if present, are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; or when X and/or Y is —PR^{d}R^{e} or —NHPR^{d}R^{e}, R^{d} and R^{e} together with the heteroatom to which they are attached form a heterocycle.

In preferred processes of the present invention, the Group 8 transition metal catalyst comprises a tridentate ligand having a formula (D)
wherein:
X is selected from —SR^{d} and —PR^{d}R^{e};
R²⁰ and R^{x} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
or X is a heteroatom and when taken together with R²⁰ it forms an optionally substituted heteroaromatic ring when R^{x} is absent, wherein the heteroaromatic ring is a nitrogen-containing heteroaromatic ring selected from pyridinyl, pyrrolyl, imidazolyl, pyrazolyl, triazolyl, tetrazolyl, and pyrimidyl;
Y is selected from —SR^{d} and —PR^{d}R^{e};
R²¹ and R^{y} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
or Y is a heteroatom and when taken together with R²¹ it forms an optionally substituted heteroaromatic ring when R^{y} is absent, wherein the heteroaromatic ring is a nitrogen-containing heteroaromatic ring selected from pyridinyl, pyrrolyl, imidazolyl, pyrazolyl, triazolyl, tetrazolyl, and pyrimidyl;
R^{22a}, R^{22b}, R^{23a}, R^{23b} and R²⁴ are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
each q and s is independently 1 or 2; and
R^{d} and R^{e}, if present, are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, unsubstituted C₂₋₂₀-heterocycloalkyl, substituted or unsubstituted C₆₋₂₀-aryl, and substituted or unsubstituted C₄₋₂₀-heteroaryl; or when X and/or Y is —PR^{d}R^{e}, R^{d} and R^{e} together with the heteroatom to which they are attached form a heterocycle.

In preferred processes of the present invention, the Group 8 transition metal catalyst comprises a tridentate ligand having a formula (D), wherein:
X is —SR^{d};
R²⁰ and R^{x} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
Y is —SR^{d};
R²¹ and R^{y} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
R^{22a}, R^{22b}, R^{23a}, R^{23b} and R²⁴ are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
each q and s is independently 1 or 2; and
R^{d} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl.

Preferably, the Group 8 transition metal catalyst comprises a tridentate ligand having a formula (D), wherein:
X is —SR^{d};
R²⁰ and R^{x} are each independently selected from hydrogen, substituted or unsubstituted C₁₋₂₀-alkyl, substituted or unsubstituted C₁₋₂₀-heteroalkyl, and substituted or unsubstituted C₃₋₂₀-cycloalkyl;
Y is —SR^{d};
R²¹ and R^{y} are each independently selected from hydrogen, substituted or unsubstituted C₁₋₂₀-alkyl, substituted or unsubstituted C₁₋₂₀-heteroalkyl, and substituted or unsubstituted C₃₋₂₀-cycloalkyl;
R^{22a}, R^{22b}, R^{23a}, R^{23b} and R²⁴ are each independently selected from hydrogen, substituted or unsubstituted C₁₋₂₀-alkyl, substituted or unsubstituted C₁₋₂₀-heteroalkyl, and substituted or unsubstituted C₃₋₂₀-cycloalkyl;
each q and s is independently 1 or 2; and
R^{d} are each independently selected from hydrogen, substituted or unsubstituted C₁₋₂₀-alkyl, substituted or unsubstituted C₁₋₂₀-heteroalkyl, and substituted or unsubstituted C₃₋₂₀-cycloalkyl.

More preferably, the Group 8 transition metal catalyst comprises a tridentate ligand having a formula (D), wherein:
X is —SR^{d};
R²⁰ and R^{x} are each independently selected from hydrogen and substituted or unsubstituted C₁₋₂₀-alkyl;
Y is —SR^{d};
R²¹ and R^{y} are each independently selected from hydrogen and substituted or unsubstituted C₁₋₂₀-alkyl;
R^{22a}, R^{22b}, R^{23a}, R^{23b} and R²⁴ are each independently selected from hydrogen and substituted or unsubstituted C₁₋₂₀-alkyl;
each q and s is independently 1 or 2; and
R^{d} are each independently selected from hydrogen and substituted or unsubstituted C₁₋₂₀-alkyl, substituted or unsubstituted C₁₋₂₀-heteroalkyl, and substituted or unsubstituted C₃₋₂₀-cycloalkyl.

Even more preferably, the Group 8 transition metal catalyst comprises a tridentate ligand having a formula (D), wherein:
X and Y are each —SR^{d};
R²⁰, R^{x}, R²¹, R^{y}, R²², R^{22b}, R^{23a}, R^{23b} and R²⁴ are each hydrogen;
q and s are each 1; and
R^{d} are each independently substituted or unsubstituted C₁₋₂₀-alkyl, preferably C₁₋₁₀ alkyl.

Even more preferably, the Group 8 transition metal catalyst comprises a tridentate ligand having a formula (D), wherein:
X and Y are each —SEt;
R²⁰, R^{x}, R²¹, R^{y}, R^{22a}, R^{22b}, R^{23a}, R^{23b} and R²⁴ are each hydrogen; and
q and s are each 1.

In alternative preferred processes of the present invention, the Group 8 transition metal catalyst comprises a tridentate ligand having a formula (D), wherein:
X is a heteroatom and when taken together with R²⁰ it forms an optionally substituted heteroaromatic ring when R^{x} is absent;
Y is —PR^{d}R^{e};
R²¹ and R^{y} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
R^{22a}, R^{22b}, R^{23a}, R^{23b} and R²⁴ are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
each q and s is independently 1 or 2; and
R^{d} and R^{e} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; or R^{d} and R^{e} together with the heteroatom to which they are attached form a heterocycle.

Preferably, the Group 8 transition metal catalyst comprises a tridentate ligand having a formula (D), wherein:
X is a nitrogen atom and when taken together with R²⁰ it forms an optionally substituted heteroaromatic ring when R^{x} is absent, wherein the heteroaromatic ring is a nitrogen-containing heteroaromatic ring;
Y is —PR^{d}R^{e};
R²¹ and R^{y} are each independently is selected from hydrogen, substituted or unsubstituted C₁₋₂₀-alkyl, substituted or unsubstituted C₁₋₂₀-heteroalkyl, and substituted or unsubstituted C₃₋₂₀- cycloalkyl;
R^{22a}, R^{22b}, R^{23a}, R^{23b} and R²⁴ are each independently selected from hydrogen, substituted or unsubstituted C₁₋₂₀-alkyl, substituted or unsubstituted C₁₋₂₀-heteroalkyl, substituted or unsubstituted C₃₋₂₀-cycloalkyl;
each q and s is independently 1 or 2; and
R^{d} and R^{e} are each independently selected from substituted or unsubstituted C₁₋₂₀-alkyl, substituted or unsubstituted C₁₋₂₀-heteroalkyl, substituted or unsubstituted C₃₋₂₀-cycloalkyl, substituted or unsubstituted C₆₋₂₀-aryl, and substituted or unsubstituted C₄₋₂₀-heteroaryl.

More preferably, the Group 8 transition metal catalyst comprises a tridentate ligand having a formula (D), wherein:
X is a nitrogen atom and when taken together with R²⁰ it forms an optionally substituted heteroaromatic ring when R^{x} is absent, wherein the heteroaromatic ring is a nitrogen-containing heteroaromatic ring selected from pyridinyl, pyrrolyl, imidazolyl, pyrazolyl, thiazolyl, isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl;
Y is —PR^{d}R^{e};
R²¹, R^{y}, R^{22a}, R^{22b}, R^{23a}, R^{23b} and R²⁴ are each hydrogen;
each q and s is 1; and
R^{d} and R^{e} are each independently selected from substituted or unsubstituted C₁₋₂₀-alkyl and substituted or unsubstituted C₆₋₂₀-aryl.

Even more preferably, the Group 8 transition metal catalyst comprises a tridentate ligand having a formula (D), wherein:
X is a nitrogen atom and when taken together with R²⁰ it forms an optionally substituted heteroaromatic ring when R^{x} is absent, wherein the heteroaromatic ring is a nitrogen-containing heteroaromatic ring selected from pyridinyl, pyrrolyl, imidazolyl, pyrazolyl, triazolyl, tetrazolyl, and pyrimidyl;
Y is —PR^{d}R^{e};
R²¹, R^{y}, R^{22a}, R^{22b}, R^{23a}, R^{23b} and R²⁴ are each hydrogen;
each q and s is 1; and
R^{d} and R^{e} are each independently selected from methyl, ethyl, n-propyl, iso-propyl, n-butyl, sec-butyl, tert-butyl, pentyl, hexyl, phenyl, tolyl, xylyl, and methoxyphenyl.

Even more preferably, the Group 8 transition metal catalyst comprises a tridentate ligand having a formula (D), wherein:
X is a nitrogen atom and when taken together with R²⁰ it forms an optionally substituted pyridinyl ring when R^{x} is absent;
Y is —PR^{d}R^{e};
R²¹, R^{y}, R^{22a}, R^{22b}, R^{23a}, R^{23b} and R²⁴ are each hydrogen;
each q and s is 1; and
R^{d} and R^{e} are each independently selected from methyl, ethyl, iso-propyl, tert-butyl, phenyl, tolyl, xylyl, and methoxyphenyl.

Even more preferably, the Group 8 transition metal catalyst comprises a tridentate ligand having a formula (D), wherein:
X is a nitrogen atom and when taken together with R²⁰ it forms an optionally substituted pyridinyl ring when R^{x} is absent;
Y is —PR^{d}R^{e};
R²¹, R^{y}, R^{22a}, R^{22b}, R^{23a}, R^{23b} and R²⁴ are each hydrogen;
each q and s is 1; and
R^{d} and R^{e} are each phenyl.

In preferred processes of the present invention, the Group 8 transition metal catalyst is of formula (E) or formula (F)

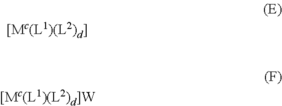

wherein:
M^{c} is a Group 8 transition metal, preferably ruthenium;
L¹ is a tridentate ligand of formula (D) as hereinbefore defined;
L² are ligands which may be the same or different;
d is 1, 2 or 3; and
W is a non-coordinated anionic ligand.

In preferred processes of the present invention, d is 3.

As will be understood by a skilled person, each L² may be a monodentate ligand or a multidentate ligand, provided the combination of L² ligands is allowed by the rules of valency. In preferred processes of the present invention, each L² is a monodentate ligand. Preferably, each L² is independently a neutral monodentate ligand or an anionic monodentate ligand. In preferred processes of the present invention, each L² is independently selected from —H, —CO, —CN, —P(R″′)₃, —As(R″′)₃, —CR″′, —OR″′, —O(C═O)R″′, —NR″′₂, halogen (e.g. —Cl, —Br, —I), and solvent, wherein each R″′ is independently selected from substituted or unsubstituted alkyl, substituted or unsubstituted alkenyl, substituted or unsubstituted alkynyl, substituted or unsubstituted heteroalkyl, substituted or unsubstituted alkoxy, substituted or unsubstituted cycloalkyl, substituted or unsubstituted heterocycloalkyl, substituted or unsubstituted aryl, and substituted or unsubstituted heteroaryl. Preferably, each L² is independently selected from —H, —CO, —P(R′′)₃, and halogen. More preferably, each L² is independently selected from —CO, —PPh₃, and —Cl. When L² is solvent, the solvent is preferably selected from THF, Me-THF, MeCN, H₂O and an alcohol (e.g. methanol, ethanol, iso-propanol etc.).

In the Group 8 transition metal catalysts of formula (F), Wis a non-coordinated anionic ligand. By “non-coordinated anion ligand”, we mean the anionic ligand is forced to the outer sphere of the metal centre. The anionic ligand, therefore, is dissociated from the metal centre. This is in contrast to neutral complexes in which the anionic ligand is bound to the metal within the coordination sphere. The anionic ligand can be generally identified as non-coordinating by analysing the X-ray crystal structure of the cationic complex. Preferably, Wis selected from the group consisting of triflate (i.e. TfO⁻ or CF₃SO₃⁻), tetrafluoroborate (i.e. ⁻BF₄), hexafluoroantimonate (i.e. ⁻SbF₆), hexafluorophosphate (PF₆⁻), [B[3,5-(CF₃)₂C₆H₃]₄]⁻ ([BAr^{F}₄]⁻), halide (e.g. Cl⁻, Br⁻, I⁻) and mesylate (MsO⁻ or MeSO₃⁻).

Preferably, the Group 8 transition metal catalyst is a transition metal catalyst of formula (E).

Alternatively, the Group 8 transition metal catalyst is a transition metal catalyst of formula (F).

In preferred processes of the present invention, the Group 8 transition metal catalyst is

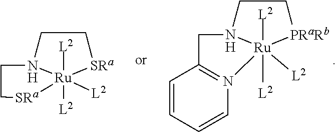

In preferred processes of the present invention, the Group 8 transition metal catalyst is Ru-SNS or Ru-PNN. In particularly preferred processes of the present invention, the Group 8 transition metal catalyst is Ru-SNS.

In preferred processes of the present invention, the Group 8 transition metal catalyst is

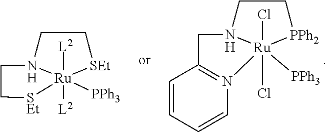

In particularly preferred processes of the present invention, the Group 8 transition metal catalyst is

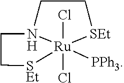

In preferred processes of the present invention, the Group 8 transition metal catalyst is present in an amount of 0.1 to 5.0 mol % based upon the total amount of compound (III), preferably 0.5 to 4.0 mol % based upon the total amount of compound (III), more preferably 1.0 to 3.0 mol % based upon the total amount of compound (III) (e.g. 2.0 mol % based upon the total amount of compound (III)).

In preferred processes of the present invention, the base used in the reaction to prepare a compound of formula (IV) from a compound of formula (III) is a metal alkoxide. The metal alkoxide is preferably a metal methoxide, a metal ethoxide, a metal iso-propoxide, or a metal tert-butoxide.

In preferred processes of the present invention, the base is an alkali metal alkoxide. The alkali metal alkoxide is preferably an alkali metal methoxide, an alkali metal ethoxide, an alkali metal iso-propoxide, or an alkali metal tert-butoxide. The alkali metal alkoxide is more preferably an alkali metal tert-butoxide.

Preferred metal alkoxides include sodium tert-butoxide or potassium tert-butoxide, preferably potassium tert-butoxide.

In preferred processes of the present invention, the base used in the reaction to prepare a compound of formula (IV) from a compound of formula (III) is present in an amount of 10 to 100 mol % based upon the total amount of said compound of formula (III), preferably 20 to 90 mol % based upon the total amount of said compound of formula (III), more preferably 30 to 80 mol % based upon the total amount of said compound of formula (III) (e.g. 50 mol % based upon the total amount of said compound of formula (III)).

In preferred processes of the present invention, R¹ in formula (III) is a substituted or unsubstituted C₁₋₁₀ straight-chain or C₃₋₁₀ branched-chain alkyl group or a substituted or unsubstituted C₆₋₁₀ aryl group. Particularly preferred C₁₋₁₀-alkyl groups include methyl, ethyl, n-propyl, iso-propyl, n-butyl, sec-butyl, tert-butyl, pentyl, and hexyl, more preferably methyl, ethyl, iso-propyl, tert-butyl, even more preferably ethyl. Preferred C₆₋₁₀-aryl groups include phenyl, tolyl, xylyl, and methoxyphenyl, more preferably phenyl.

In preferred processes of the present invention, the compound of formula (III) is reduced under a hydrogen pressure in the range 1 to 50 bar, preferably 10 to 40 bar, more preferably 20 to 30 bar (e.g. 28 bar). As will be understood by a skilled person, the hydrogen pressure of the reduction reaction may be varied within these ranges during the reaction.

In preferred processes of the present invention, the compound of formula (III) is reduced at a temperature in the range 10 to 150° C., preferably 30 to 140° C., more preferably 50 to 130° C., more preferably 65 to 120° C. As will be understood by a skilled person, the temperature of the reduction reaction may be varied within these ranges during the reaction. Thus, in preferred processes of the present invention, the compound of formula (III) is reduced at a temperature in the range 50 to 70° C. (e.g. 65° C.) for a period of time, and then at a temperature in the range 110 to 140° C. (e.g. 120° C.) for a further period of time.

In preferred processes of the present invention, the compound of formula (III) is reduced for a duration of 1.5 to 10 hours, preferably 5 to 9 hours, more preferably 6 to 8 hours.

In preferred processes of the present invention, the compound of formula (III) is prepared by reacting a compound of formula (I)

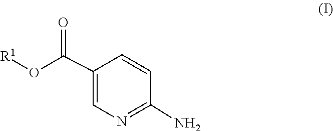

wherein R¹ is as hereinbefore defined,
with a compound of formula (II)

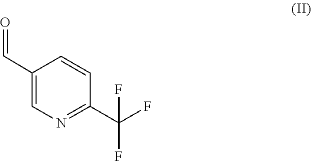 [SMILES: O=Cc1ccc(C(F)(F)F)nc1]

in the presence of an acid catalyst.

The acid catalyst may be an inorganic acid catalyst or an organic acid catalyst.

In preferred processes of the present invention, the acid catalyst is an inorganic acid catalyst. Suitable inorganic acid catalysts include hydrochloric acid, hydrobromic acid, hydroiodic acid, sulfuric acid, phosphoric acid, nitric acid, and boric acid.

In preferred processes of the present invention, the acid catalyst is an organic acid catalyst. Preferably, the organic acid catalyst is an organic sulfonic acid or a carboxylic acid. Suitable organic sulfonic acids include methanesulfonic acid, p-toluenesulfonic acid, and p-aminobenzenesulfonic acid. Suitable carboxylic acids include formic acid, acetic acid, and trifluoroacetic acid.

In preferred processes of the present invention, the acid catalyst is trifluoroacetic acid.

In preferred processes of the present invention, the acid catalyst is present in an amount of 5 to 15 mol % based upon the total amount of the compound of formula (I), preferably 8 to 12 mol % based upon the total amount of the compound of formula (I) (e.g. 10 mol % based upon the total amount of the compound of formula (I)).

In preferred processes of the present invention, the reacting of the compound of formula (I) and the compound of formula (II) takes place in the presence of a solvent. In preferred processes of the present invention, the solvent is selected from benzene, toluene, o-xylene, m-xylene, p-xylene. In particularly preferred processes of the present invention, the solvent is toluene.

In preferred processes of the present invention, the reacting of the compound of formula (I) and the compound of formula (II) is conducted at a temperature in the range 10 to 150° C., preferably 30 to 140° C., more preferably 50 to 130° C., more preferably 65 to 120° C.

In preferred processes of the present invention, the reacting of the compound of formula (I) and the compound of formula (II) is conducted for a duration of 4 to 10 hours, preferably 5 to 9 hours, more preferably 6 to 8 hours.

In preferred processes of the present invention, the compound of formula (IX) is prepared by reacting a compound of formula (VIII)

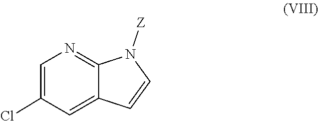

wherein
Z is —SO₂R″ or —CO₂R³;
R″ is a substituted or unsubstituted C₁₋₂₀ straight-chain or C₃₋₂₀ branched-chain alkyl group or a substituted or unsubstituted C₆₋₂₀ aryl group;
R³ is a substituted or unsubstituted C₁₋₂₀ straight-chain or C₃₋₂₀ branched-chain alkyl group or a substituted or unsubstituted C₆₋₂₀ aryl group;
with a borylation agent in the presence of a Group 9 transition metal catalyst.

Conventional borylation reactions involving azaindole-based compounds are typically carried out in three separate steps. The first step involves protection of the nitrogen atom in the five-membered ring, the second step involves halogenating the azaindole and the third step involves coupling this halogenated compound with the desired borylation agent. The processes of the present invention are therefore more efficient because the borylation occurs in a single step.

The processes of the present invention employ a Group 9 transition metal catalyst. Preferably, the Group 9 transition metal catalyst is a cobalt catalyst, a rhodium catalyst, or an iridium catalyst. More preferably, the Group 9 transition metal catalyst is a rhodium catalyst or an iridium catalyst. Most preferably, the Group 9 transition metal catalyst is an iridium catalyst.

In preferred processes of the present invention, the Group 9 transition metal catalyst comprises a bidentate ligand having a formula (G)

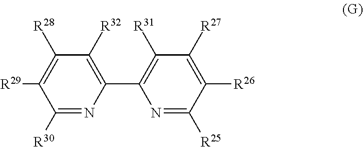

wherein:
each of R²⁵, R²⁶, R²⁷, R²⁸, R²⁹, and R³⁰ are independently selected from hydrogen, a substituted or unsubstituted C₁₋₂₀ straight-chain or C₃₋₂₀ branched-chain alkyl group or a substituted or unsubstituted C₆₋₂₀ aryl group;
each of R³¹ and R³² is hydrogen; or, together with the atoms to which they are attached, R³¹ and R³² form a ring.

In preferred bidentate ligands of formula (G), each of R²⁵, R²⁶, R²⁷, R²⁸, R²⁹, and R³⁰ are preferably independently selected from hydrogen, a substituted or unsubstituted C₁₋₁₀ straight-chain or C₃₋₁₀ branched-chain alkyl group or a substituted or unsubstituted C₆₋₁₀ aryl group. Preferred C₁₋₁₀-alkyl groups include methyl, ethyl, n-propyl, iso-propyl, n-butyl, sec-butyl, tert-butyl, pentyl, and hexyl, more preferably methyl, ethyl, iso-propyl, and tert-butyl, even more preferably methyl and tert-butyl. Preferred C₆₋₁₀-aryl groups include phenyl, tolyl, xylyl, and methoxyphenyl, more preferably phenyl.

In preferred bidentate ligands of formula (G), each of R³¹ and R³² is hydrogen.

In alternative preferred bidentate ligands of formula (G), together with the atoms to which they are attached, R³¹ and R³² form a ring. Preferably, the ring is a six-membered ring. More preferably, the ring is a six-membered carbocyclic ring. Alternatively, the ring is a six-membered heterocyclic ring.

Particularly preferred bidentate ligands of formula (G) include 1,10-phenanthroline, 3,4,7,8-tetramethyl-1,10-phenanthroline, 4,7-dimethyl-1,10-phenanthroline, 5,6-dimethyl-1,10-phenanthroline, 2,9-dimethyl-1,10-phenanthroline, 2,2′-bipyridine, 4,4′-di-methyl-2,2′-bipyridine and 4,4′-di-tert-butyl-2,2′-bipyridine.

In preferred processes of the present invention, the Group 9 transition metal catalyst is of formula (H)

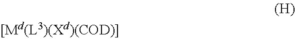

wherein:
M^{d} is a Group 9 transition metal, preferably iridium;
L³ is a bidentate ligand of formula (G) as hereinbefore defined;
X^{d} is an anionic ligand; and
COD is cyclooctadiene.

In preferred Group 9 transition metal catalysts of formula (H), X^{d} is a halo group, such as —Cl, —Br, and —I, an alkoxy group, or trifluoroacetate (i.e. F₃CCO₂⁻). Preferably, X^{d} is —Cl.

In preferred processes of the present invention, the Group 9 transition metal catalyst is chloro(1,5-cyclooctadiene)(1,10-phenanthroline)iridium(I), chloro(1,5-cyclooctadiene)(3,4,7,8-tetramethyl-1,10-phenanthroline)iridium(I) chloro(1,5-cyclooctadiene)(4,7-dimethyl-1,10-phenanthroline)iridium(I), chloro(1,5-cyclooctadiene)(5,6-dimethyl-1,10-phenanthroline)iridium(I), chloro(1,5-cyclooctadiene)(2,9-dimethyl-1,10-phenanthroline)iridium(I), chloro(1,5-cyclooctadiene)(2,2′-bipyridine)iridium(I), chloro(1,5-cyclooctadiene)(4,4′-di-methyl-2,2′-bipyridine)iridium(I), or chloro(1,5-cyclooctadiene)(4,4′-di-tert-butyl-2,2′-bipyridine)iridium(I). In particularly preferred processes of the present invention, the Group metal 9 transition catalyst is chloro(1,5-cyclooctadiene)(1,10-phenanthroline)iridium(I).

As will be understood by a skilled person, the Group 9 transition metal catalyst may be formed in situ. As will also be understood by a skilled person, the active catalytic species may also be formed in situ.

The Group 9 transition metal catalysts may be in the form of an adduct with a solvent, such as THF, dioxane, diethyl ether, or cyclopentyl methyl ether. Preferably, the Group 9 transition metal catalysts are in the form of an adduct with THF. Thus, in preferred processes of the present invention, the Group 9 transition metal catalyst is chloro(1,5-cyclooctadiene)(1,10-phenanthroline)iridium(I)·THF adduct, chloro(1,5-cyclooctadiene)(3,4,7,8-tetramethyl-1,10-phenanthroline)iridium(I)·THF adduct, chloro(1,5-cyclooctadiene)(4,7-dimethyl-1, 10-phenanthroline)iridium(I)·THF adduct, chloro(1,5-cyclooctadiene)(5,6-dimethyl-1,10-phenanthroline)iridium(I)·THF adduct, chloro(1,5-cyclooctadiene)(2,9-dimethyl-1,10-phenanthroline)iridium(I)·THF adduct, chloro(1,5-cyclooctadiene)(2,2′-bipyridine)iridium(I)·THF adduct, chloro(1,5-cyclooctadiene)(4,4′-di-methyl-2,2′-bipyridine)iridium(I)·THF adduct, or chloro(1,5-cyclooctadiene)(4,4′-di-tert-butyl-2,2′-bipyridine)iridium(I)·THF adduct. In particularly preferred processes of the present invention, the Group 9 transition metal catalyst is chloro(1,5-cyclooctadiene)(1,10-phenanthroline)iridium(I)·THF adduct.

In preferred processes of the present invention, the Group 9 transition metal catalyst is present in an amount of 0.01 to 1.0 mol % based upon the total amount of compound (VIII), preferably 0.05 to 0.8 mol % based upon the total amount of compound (VIII), preferably 0.1 to 0.5 mol % based upon the total amount of compound (VIII), more preferably 0.1 to 0.25 mol % based upon the total amount of compound (VIII). In the processes of the present invention, Z in formula (VIII) is —SO₂R″ or —CO₂R³. Such Z groups aid the isolation and purification of the compound of formula (IX) because they typically cause the compound of formula (IX) to be solid, meaning it can be easily filtered or crystallised. Furthermore, it is also thought that the presence of such Z groups in the compound of formula (VIII) helps to improve the yield of the borylation reaction that forms the compound of formula (IX). Without wishing to be bound by theory, this is thought to be due to (i) the Z group's ability to direct the catalyst; and (ii) the Z group's steric bulk, which ensures correct regioselectivity in the product as a result of steric interactions between the catalyst and substrate.

In preferred processes of the present invention, Z in formula (VIII) is —CO₂R³. Preferably, R³ is a substituted or unsubstituted C₁₋₁₀ straight-chain or C₃₋₁₀ branched-chain alkyl group or a substituted or unsubstituted C₆₋₁₀ aryl group. Particularly preferred C₁₋₁₀-alkyl groups include methyl, ethyl, n-propyl, iso-propyl, n-butyl, sec-butyl, tert-butyl, pentyl, and hexyl, more preferably methyl, ethyl, iso-propyl, tert-butyl, even more preferably tert-butyl. Preferred C₆₋₁₀-aryl groups include phenyl, tolyl, xylyl, and methoxyphenyl, more preferably phenyl.

In preferred processes of the present invention, Z in formula (VIII) is —SO₂R″. Preferably, R″ is a substituted or unsubstituted C₁₋₁₀ straight-chain or C₃₋₁₀ branched-chain alkyl group or a substituted or unsubstituted C₆₋₁₀ aryl group. Particularly preferred C₁₋₁₀-alkyl groups include methyl, trifluoromethyl, ethyl, n-propyl, iso-propyl, n-butyl, sec-butyl, tert-butyl, pentyl, and hexyl, more preferably methyl, ethyl, iso-propyl, and tert-butyl, even more preferably methyl and ethyl. Preferred C₆₋₁₀-aryl groups include phenyl, tolyl, xylyl, methoxyphenyl, bromophenyl, and nitrophenyl, more preferably tolyl.

In preferred processes of the present invention, the compound of formula (VIII) is reacted at a temperature in the range 5 to 100° C., preferably 10 to 90° C., more preferably 20 to 80° C., more preferably 50 to 70° C.

In preferred processes of the present invention, the compound of formula (VIII) is reacted for a duration of 0.5 to 2 hours, preferably 1 to 1.5 hours.

In preferred processes of the present invention, the borylation agent is a compound of formula (J) or formula (K):

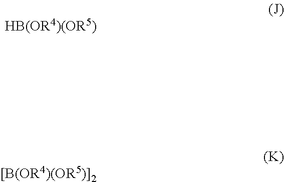

wherein in the compound of formula (J), R⁴ and R⁵ are, independently, H or a substituted or unsubstituted organic group comprising 1-20 carbon atoms; or, together with the atoms to which they are attached, R⁴ and R⁵ form a ring;
wherein in the compound of formula (K), each R⁴ and R⁵ are, independently, H or a substituted or unsubstituted organic group comprising 1-20 carbon atoms; or, together with the atoms to which they are attached, each instance of R⁴ and R⁵ can form a ring.

In preferred processes of the present invention, the borylation agent is a compound of formula (J), wherein R⁴ and R⁵ are, independently, H or a substituted or unsubstituted C₁₋₂₀ straight-chain or C₃₋₂₀ branched-chain alkyl group; or, together with the atoms to which they are attached, R⁴ and R⁵ form a ring.

In preferred processes of the present invention, the borylation agent is a compound of formula (J), wherein R⁴ and R⁵ are, independently, H or a substituted or unsubstituted C₁₋₁₀ straight-chain or C₃₋₁₀ branched-chain alkyl group, more preferably H or a substituted or unsubstituted C₁₋₅ straight-chain or C₃₋₅ branched-chain alkyl group. For example, the borylation agent may be bis(3-ethyl-3-pentoxy)borane.

In alternative preferred processes of the present invention, the borylation agent is a compound of formula (J), wherein, together with the atoms to which they are attached, R⁴ and R⁵ form a ring. For example, the borylation agent may be HBpin or HBcat, preferably HBpin.

In preferred processes of the present invention, the borylation agent is a compound of formula (K), wherein R⁴ and R⁵ are, independently, H or a substituted or unsubstituted C₁₋₂₀ straight-chain or C₃₋₂₀ branched-chain alkyl group; or, together with the atoms to which they are attached, each instance of R⁴ and R⁵ can form a ring.

In preferred processes of the present invention, the borylation agent is a compound of formula (K), wherein R⁴ and R⁵ are, independently, H or a substituted or unsubstituted C₁₋₁₀ straight-chain or C₃₋₁₀ branched-chain alkyl group, more preferably H or a substituted or unsubstituted C₁₋₅ straight-chain or C₃₋₅ branched-chain alkyl group, even more preferably H. For example, the borylation agent may be (dihydroxyboranyl)boronic acid.

In alternative preferred processes of the present invention, the borylation agent is a compound of formula (K), wherein, together with the atoms to which they are attached, each instance of R⁴ and R⁵ forms a ring. For example, the borylation agent may be B₂pin₂ or bis(catecholato)diboron, preferably B₂pin₂.

In preferred processes of the present invention, the borylation agent is selected from B₂pin₂, HBpin, (dihydroxyboranyl)boronic acid, HBcat, and bis(catecholato)diboron. More preferably, the borylation agent is selected from HBpin and B₂pin₂. Most preferably, the borylation agent is B₂pin₂.

In preferred processes of the present invention, the borylation agent is present in an amount of 70 mol % or more based upon the total amount of said compound of formula (VIII), more preferably 80 mol % or more based upon the total amount of said compound of formula (VIII), more preferably 90 mol % or more based upon the total amount of said compound of formula (VIII), more preferably 100 mol % or more based upon the total amount of said compound of formula (VIII).

In preferred processes of the present invention, the borylation agent is present in an amount of 70 to 200 mol % based upon the total amount of said compound of formula (VIII), more preferably 70 to 175 mol % based upon the total amount of said compound of formula (VIII), more preferably 70 to 150 mol % based upon the total amount of said compound of formula (VIII), more preferably 70 to 125 mol % based upon the total amount of said compound of formula (VIII), more preferably 70 to 100 mol % based upon the total amount of said compound of formula (VIII), more preferably 70 to 90 mol % based upon the total amount of said compound of formula (VIII), even more preferably 70 to 80 mol % based upon the total amount of said compound of formula (VIII).

In preferred processes of the present invention, the compound of formula (VIII) is prepared by reacting a compound of formula (VII)

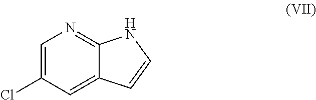

with a sulfonyl halide, of formula R″SO₂X^{a} wherein X^{a} is a halide and R″ is as hereinbefore defined, or a compound of formula (R³O(CO))₂O, wherein R³ is as hereinbefore defined.

In preferred processes of the present invention, the compound of formula (R³O(CO))₂O is selected from (MeO(CO))₂O, (EtO(CO))₂O, (ⁱPrO(CO))₂O, and (ⁱBuO(CO))₂O. Most preferably, the compound of formula (R³O(CO))₂O is (^{t}BuO(CO))₂O.

In preferred processes of the present invention, the sulfonyl halide of formula R″SO₂X^{a} is selected from a benzenesulfonyl halide, a brosyl halide, a tosyl halide, a nosyl halide, a mesyl halide, a triflyl halide, and an ethanesulfonyl halide. In particularly preferred processes of the present invention, the sulfonyl halide of formula R″SO₂X^{a} is selected from benzenesulfonyl chloride, brosyl chloride, tosyl chloride, nosyl chloride, mesyl chloride, triflyl chloride, and ethanesulfonyl chloride. Most preferably, the sulfonyl halide of formula R″SO₂X^{a} is tosyl chloride.

In preferred processes of the present invention, the sulfonyl halide of formula R″SO₂X^{a} or the compound of formula (R³O(CO))₂O is present in an amount of 110 to 150 mol % based upon the total amount of said compound of formula (VII), preferably 110 to 120 mol % based upon the total amount of said compound of formula (VII).

In preferred processes of the present invention, the compound of formula (VII) is reacted with a sulfonyl halide of formula R″SO₂X^{a} or a compound of formula (R³O(CO))₂O in the presence of a base. Preferably, the base is an organic base. More preferably, the base is an organic amine. Even more preferably, the base is selected from N-N-dimethylaniline, triethylamine, Et₂ⁱPrN, pyridine, DMAP, DABCO and DBU. Most preferably, the base is N—N-dimethylaniline.

In preferred processes of the present invention, the base is present in an amount of 1.0 to 4.0 mol % based upon the total amount of said compound of formula (VII), preferably 2.0 to 3.0 mol % based upon the total amount of said compound of formula (VII).

In preferred processes of the present invention, the compound of formula (VII) is reacted at room temperature.

In preferred processes of the present invention, the compound of formula (VII) is reacted for a duration of 0.5 to 2 hours, preferably 1 to 1.5 hours.

The present invention is also directed to a process for the preparation of a compound of formula (VI), or a salt or derivative thereof,

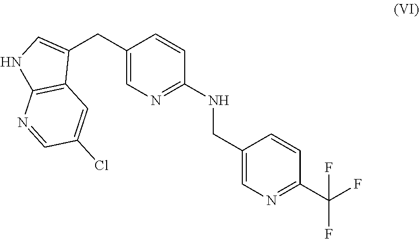 [SMILES: FC(F)(F)c1ccc(CNc2ccc(Cc3c[nH]c4ncc(Cl)cc34)cn2)cn1]

comprising:
reacting a compound of formula (I)

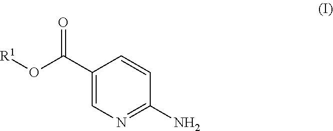

wherein R¹ is a substituted or unsubstituted C₁₋₂₀ straight-chain or C₃₋₂₀ branched-chain alkyl group or a substituted or unsubstituted C₆₋₂₀ aryl group,
with a compound of formula (II),

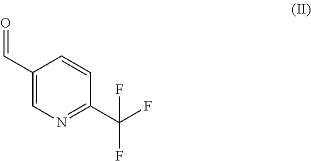 [SMILES: O=Cc1ccc(C(F)(F)F)nc1]

in the presence of an acid catalyst, to give a compound of formula (III),

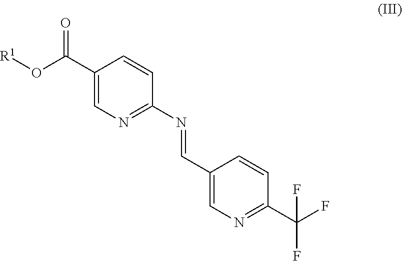

wherein R¹ is as hereinbefore defined;
reducing said compound of formula (III) with hydrogen in the presence of a Group 8 transition metal catalyst and a first base to give a compound of formula (IV)

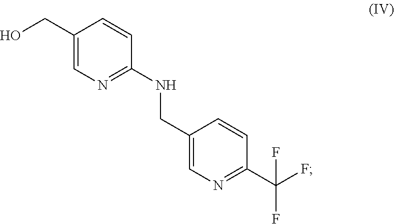 [SMILES: OCc1ccc(NCc2ccc(C(F)(F)F)nc2)nc1]

reacting said compound of formula (IV), or a salt thereof, with a dialkyl carbonate of formula R²O(CO)OR², wherein each R² is a substituted or unsubstituted C₁₋₂₀ straight-chain or C₃₋₂₀ branched-chain alkyl group, in the presence of a second base to give a compound of formula (V)

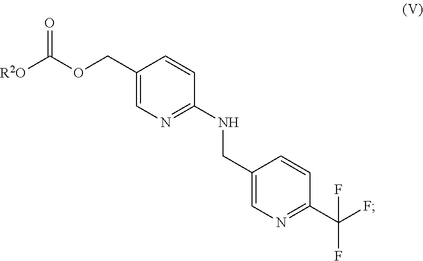

wherein R² is as hereinbefore defined;
reacting a compound of formula (VII)

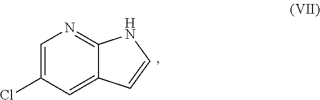

with a sulfonyl halide of formula R″SO₂X^{a} wherein X^{a} is a halide and R″ is a substituted or unsubstituted C₁₋₂₀ straight-chain or C₃₋₂₀ branched-chain alkyl group or a substituted or unsubstituted C₆₋₂₀ aryl group, or a compound of formula (R³O(CO))₂O, wherein R³ is a substituted or unsubstituted C₁₋₂₀ straight-chain or C₃₋₂₀ branched-chain alkyl group or a substituted or unsubstituted C₆₋₂₀ aryl group, to give a compound of formula (VIII)

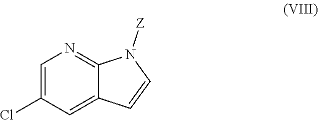

wherein Z is —SO₂R″ or —CO₂R³, and
R″ and R³ is as hereinbefore defined;
reacting said compound of formula (VIII) with a borylation agent in the presence of a Group 9 transition metal catalyst to give a compound of formula (IX)

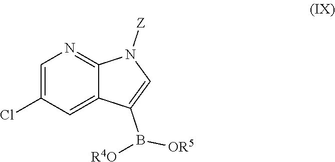

wherein Z is as hereinbefore defined,
R⁴ and R⁵ are, independently, H or a substituted or unsubstituted organic group comprising 1-20 carbon atoms; or, together with the atoms to which they are attached, R⁴ and R⁵ form a ring; and
reacting said compound of formula (V) with said compound of formula (IX) in the presence of a Group 10 transition metal catalyst, a third base, and water to give said compound of formula (VI).

In preferred processes of the present invention, the first base is a metal alkoxide. The metal alkoxide is preferably a metal methoxide, a metal ethoxide, a metal iso-propoxide, or a metal tert-butoxide.

In preferred processes of the present invention, the first base is an alkali metal alkoxide. The alkali metal alkoxide is preferably an alkali metal methoxide, an alkali metal ethoxide, an alkali metal iso-propoxide, or an alkali metal tert-butoxide. The alkali metal alkoxide is more preferably an alkali metal tert-butoxide.

Preferred metal alkoxides include sodium tert-butoxide or potassium tert-butoxide, preferably potassium tert-butoxide.

In preferred processes of the present invention, the first base is present in an amount of 10 to 100 mol % based upon the total amount of said compound of formula (III), preferably 20 to 90 mol % based upon the total amount of said compound of formula (III), more preferably 30 to 80 mol % based upon the total amount of said compound of formula (III) (e.g. 50 mol % based upon the total amount of said compound of formula (III)).

In preferred processes of the present invention, the second base is a metal alkoxide. The metal alkoxide is preferably a metal methoxide, a metal ethoxide, a metal iso-propoxide, or a metal tert-butoxide.

In preferred processes of the present invention, the second base is an alkali metal alkoxide. The alkali metal alkoxide is preferably an alkali metal methoxide, an alkali metal ethoxide, an alkali metal iso-propoxide, or an alkali metal tert-butoxide. The alkali metal alkoxide is more preferably an alkali metal methoxide or an alkali metal ethoxide, preferably an alkali metal methoxide.

Preferred metal alkoxides include sodium methoxide, sodium ethoxide, potassium methoxide, or potassium ethoxide, preferably sodium methoxide.

In preferred processes of the present invention, the second base is present in an amount of 1 to 10 mol % based upon the total amount of said compound of formula (IV) or salt thereof, preferably 2 to 9 mol % based upon the total amount of said compound of formula (IV) or salt thereof, more preferably 3 to 8 mol % based upon the total amount of said compound of formula (IV) or salt thereof (e.g. 5 mol % based upon the total amount of said compound of formula (IV) or salt thereof).

The third base must be selected to ensure that it is strong enough to allow the coupling reaction to progress, but not so strong that the carbonate compound of formula (V) will degrade in its presence. The skilled person is able to select a suitable base.

Thus, in preferred processes of the present invention, the third base is selected from a metal carbonate, a metal phosphate, a metal hydroxide, and an amine base. Preferably, the third base is selected from an alkali metal carbonate, an alkali metal phosphate, an alkali metal hydroxide, and an amine base. More preferably, the third base is selected from Na₂CO₃, K₂CO₃, Na₃PO₄, K₃PO₄, NaOH, KOH, Et₃N, Et₂ⁱPrN, DMAP, DABCO, or DBU. Most preferably, the third base is K₂CO₃.

In preferred processes of the present invention, the third base is not an alkoxide base (e.g. a metal alkoxide).

In preferred processes of the present invention, the third base is a metal carbonate, preferably an alkali metal carbonate. In particularly preferred processes of the present invention, the third base is selected from Na₂CO₃ and K₂CO₃. Most preferably, the third base is K₂CO₃.

In preferred processes of the present invention, the third base is a metal phosphate, preferably an alkali metal phosphate. In particularly preferred processes of the present invention, the third base is selected from Na₃PO₄ and K₃PO₄. Most preferably, the third base is K₃PO₄.

In preferred processes of the present invention, the third base is a metal hydroxide, preferably an alkali metal hydroxide. In particularly preferred processes of the present invention, the third base is selected from NaOH and KOH. Most preferably, the base is KOH.

In preferred processes of the present invention, the third base is an amine base. Preferably, the amine base is compound having a formula selected from R′—NH₂, R′₂NH, and R′₃N, wherein R′ are independently alkyl, aryl or heteroaryl groups, or the amine base is a cyclic amine base. In particularly preferred processes of the present invention, the third base is selected from Et₃N, Et₂ⁱPrN, DMAP, DABCO, or DBU. Most preferably, the third base is Et₃N.

In preferred processes of the present invention, the third base is present in an amount of 50 to 300 mol % based upon the total amount of the compound of formula (V), more preferably 100 to 250 mol % based upon the total amount of the compound of formula (V), more preferably 150 to 200 mol % based upon the total amount of the compound of formula (V).

Other preferred features of this process of the present invention are as described above. As will be understood by a skilled person, the steps in the process leading to the compound of formula (V) may be carried out before, after, or simultaneously with, the steps in the process leading to the compound of formula (IX).

The present invention also provides a process for the preparation of a compound of formula (VI), or a salt or derivative thereof,

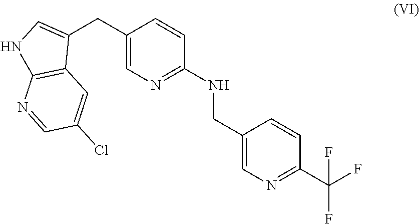 [SMILES: FC(F)(F)c1ccc(CNc2ccc(Cc3c[nH]c4ncc(Cl)cc34)cn2)cn1]

comprising:
reacting a compound of formula (I)

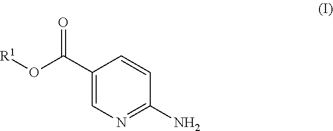

wherein R¹ is a substituted or unsubstituted C₁₋₂₀ straight-chain or C₃₋₂₀ branched-chain alkyl group or a substituted or unsubstituted C₆₋₂₀ aryl group,
with a compound of formula (II),

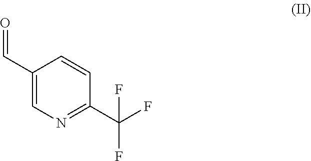 [SMILES: O=Cc1ccc(C(F)(F)F)nc1]

in the presence of an acid catalyst, to give a compound of formula (III),

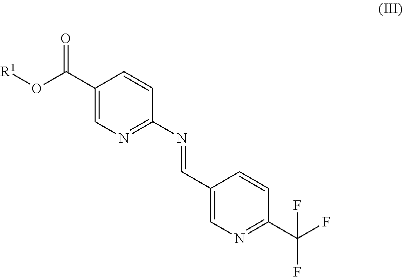

wherein R¹ is as hereinbefore defined;
reducing said compound of formula (III) with hydrogen in the presence of a Group 8 transition metal catalyst and a first base to give a compound of formula (IV)

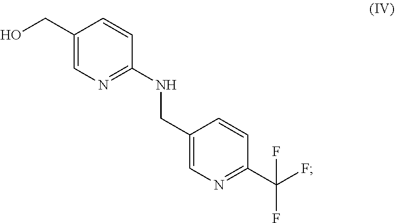 [SMILES: OCc1ccc(NCc2ccc(C(F)(F)F)nc2)nc1]

reacting a compound of formula (VII)

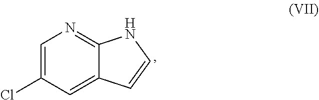

with a sulfonyl halide of formula R″SO₂X^{a}, wherein X^{a} is a halide and R″ is a substituted or unsubstituted C₁₋₂₀ straight-chain or C₃₋₂₀ branched-chain alkyl group or a substituted or unsubstituted C₆₋₂₀ aryl group, or a compound of formula (R³O(CO))₂O, wherein R³ is a substituted or unsubstituted C₁₋₂₀ straight-chain or C₃₋₂₀ branched-chain alkyl group or a substituted or unsubstituted C₆₋₂₀ aryl group, to give a compound of formula (VIII)

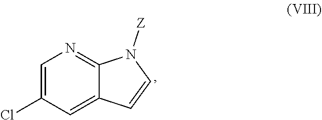

wherein Z is —SO₂R″ or —CO₂R³, wherein R″ and R³ are as hereinbefore defined;
reacting said compound of formula (VIII) with a borylation agent in the presence of a Group 9 transition metal catalyst to give a compound of formula (IX)

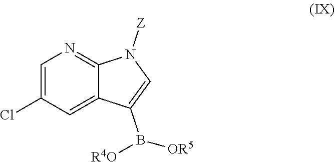

wherein Z is as hereinbefore defined,
R⁴ and R⁵ are, independently, H or a substituted or unsubstituted organic group comprising 1-20 carbon atoms; or, together with the atoms to which they are attached, R⁴ and R⁵ form a ring; and
reacting a compound of formula (V)

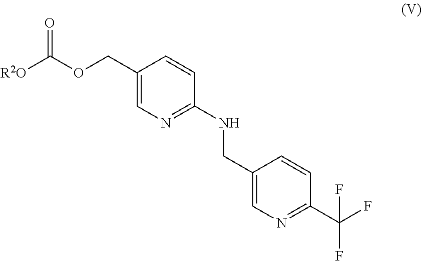

wherein R² is a substituted or unsubstituted C₁₋₂₀ straight-chain or C₃₋₂₀ branched-chain alkyl group,
with said compound of formula (IX) in the presence of a Group 10 transition metal catalyst, a third base, and water to give said compound of formula (VI),
wherein said compound of formula (V) is prepared in situ in the presence of the compound of formula (IX), the Group 10 transition metal catalyst, the third base and water by reacting said compound of formula (IV), or a salt thereof, with a dialkyl carbonate of formula R²O(CO)OR², wherein each R² is a substituted or unsubstituted C₁₋₂₀ straight-chain or C₃₋₂₀ branched-chain alkyl group.

As will be understood by a skilled person, this particular process of the present invention does not involve a second base because the compound of formula (V) is prepared in situ.

In preferred processes of the present invention, the first base is a metal alkoxide. The metal alkoxide is preferably a metal methoxide, a metal ethoxide, a metal iso-propoxide, or a metal tert-butoxide.

In preferred processes of the present invention, the first base is an alkali metal alkoxide. The alkali metal alkoxide is preferably an alkali metal methoxide, an alkali metal ethoxide, an alkali metal iso-propoxide, or an alkali metal tert-butoxide. The alkali metal alkoxide is more preferably an alkali metal tert-butoxide.

Preferred metal alkoxides include sodium tert-butoxide or potassium tert-butoxide, preferably potassium tert-butoxide.

In preferred processes of the present invention, the first base is present in an amount of 10 to 100 mol % based upon the total amount of said compound of formula (III), preferably 20 to 90 mol % based upon the total amount of said compound of formula (III), more preferably 30 to 80 mol % based upon the total amount of said compound of formula (III) (e.g. 50 mol % based upon the total amount of said compound of formula (III)).

The third base must be selected to ensure that it is strong enough to allow the coupling reaction to progress, but not so strong that the carbonate compound of formula (V) will degrade in its presence. The skilled person is able to select a suitable base.

Thus, in preferred processes of the present invention, the third base is selected from a metal alkoxide, a metal carbonate, a metal phosphate, a metal hydroxide, and an amine base. Preferably, the third base is selected from an alkali metal alkoxide, an alkali metal carbonate, an alkali metal phosphate, an alkali metal hydroxide, and an amine base. More preferably, the third base is selected from LiO^{t}Bu, NaO^{t}Bu, KO^{t}Bu, Na₂CO₃, K₂CO₃, Na₃PO₄, K₃PO₄, NaOH, KOH, Et₃N, Et₂ⁱPrN, DMAP, DABCO, or DBU. Most preferably, the third base is K₂CO₃ or KO^{t}Bu.

In preferred processes of the present invention, the third base is a metal carbonate, preferably an alkali metal carbonate. In particularly preferred processes of the present invention, the third base is selected from Na₂CO₃ and K₂CO₃. Most preferably, the third base is K₂CO₃.

In preferred processes of the present invention, the third base is a metal phosphate, preferably an alkali metal phosphate. In particularly preferred processes of the present invention, the third base is selected from Na₃PO₄ and K₃PO₄. Most preferably, the third base is K₃PO₄.

In preferred processes of the present invention, the third base is a metal hydroxide, preferably an alkali metal hydroxide. In particularly preferred processes of the present invention, the third base is selected from NaOH and KOH. Most preferably, the base is KOH.

In preferred processes of the present invention, the third base is an amine base. Preferably, the amine base is compound having a formula selected from R′—NH₂, R′₂NH, and R′₃N, wherein R′ are independently alkyl, aryl or heteroaryl groups, or the amine base is a cyclic amine base. In particularly preferred processes of the present invention, the third base is selected from Et₃N, Et₂ⁱPrN, DMAP, DABCO, or DBU. Most preferably, the third base is Et₃N.

In a particularly preferred processes of the present invention, the third base is a metal alkoxide, preferably an alkali metal alkoxide. More preferably, the third base is selected from LiO^{t}Bu, NaO^{t}Bu, and KO^{t}Bu. Most preferably, the third base is KO^{t}Bu. In preferred processes of the present invention, the third base is present in an amount of 50 to 300 mol % based upon the total amount of the compound of formula (V), more preferably 100 to 250 mol % based upon the total amount of the compound of formula (V), more preferably 150 to 200 mol % based upon the total amount of the compound of formula (V).

Other preferred features of this process of the present invention are as described above. As will be understood by a skilled person, the steps leading to the compound of formula (IV) may be carried out before, after, or simultaneously with, the steps in the process leading to the compound of formula (IX).

In preferred processes of the present invention, the compound of formula (III) is

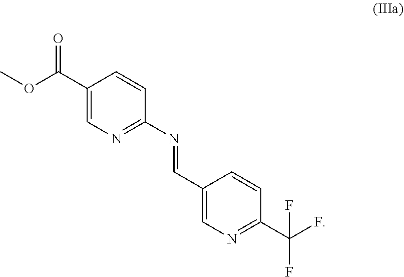 [SMILES: COC(=O)c1ccc(/N=C/c2ccc(C(F)(F)F)nc2)nc1]

In preferred processes of the present invention, the compound of formula (V) is

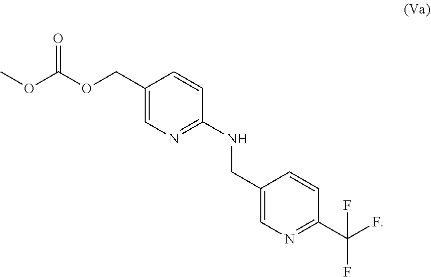 [SMILES: COC(=O)OCc1ccc(NCc2ccc(C(F)(F)F)nc2)nc1]

In preferred processes of the present invention, the compound of formula (IX) is

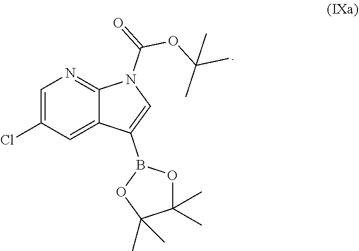 [SMILES: CC(C)(C)OC(=O)n1cc(B2OC(C)(C)C(C)(C)O2)c2cc(Cl)cnc21]

The present invention also provides a compound of formula (III), or a salt or derivative thereof,

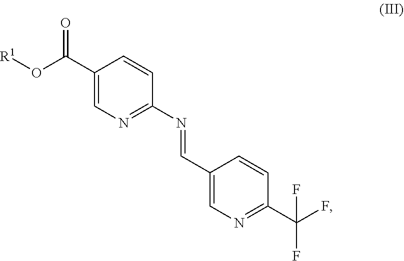

wherein R¹ is a substituted or unsubstituted C₁₋₂₀ straight-chain or C₃₋₂₀ branched-chain alkyl group or a substituted or unsubstituted C₆₋₂₀ aryl group.

In preferred compounds of the present invention, R¹ in formula (III) is a substituted or unsubstituted C₁₋₁₀ straight-chain or C₃₋₁₀ branched-chain alkyl group or a substituted or unsubstituted C₆₋₁₀ aryl group. Particularly preferred C₁₋₁₀-alkyl groups include methyl, ethyl, n-propyl, iso-propyl, n-butyl, sec-butyl, tert-butyl, pentyl, and hexyl, more preferably methyl, ethyl, iso-propyl, tert-butyl, even more preferably ethyl. Preferred C₆₋₁₀-aryl groups include phenyl, tolyl, xylyl, and methoxyphenyl, more preferably phenyl.

The present invention also provides a compound of a compound of formula (V), or a salt or derivative thereof,

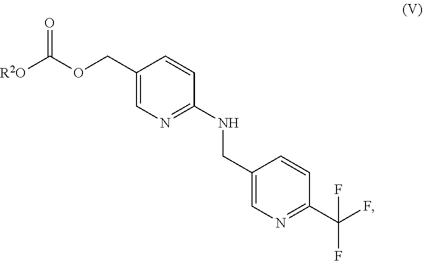

wherein R² is a substituted or unsubstituted C₁₋₂₀ straight-chain or C₃₋₂₀ branched-chain alkyl group.

In preferred compounds of the present invention, R² in formula (V) is a substituted or unsubstituted C₁₋₁₀ straight-chain or C₃₋₁₀ branched-chain alkyl group, preferably a substituted or unsubstituted C₁₋₅ straight-chain or C₃₋₅ branched-chain alkyl group. More preferably, R² in formula (V) is methyl, ethyl, iso-propyl or tert-butyl. Most preferably, R² in formula (V) is methyl.

The present invention also provides a compound of formula (IX), or a salt or derivative thereof,

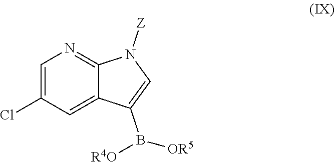

wherein Z is —SO₂R″ or —CO₂R³;
R″ is a substituted or unsubstituted C₁₋₂₀ straight-chain or C₃₋₂₀ branched-chain alkyl group or a substituted or unsubstituted C₆₋₂₀ aryl group;
R³ is a substituted or unsubstituted C₁₋₂₀ straight-chain or C₃₋₂₀ branched-chain alkyl group or a substituted or unsubstituted C₆₋₂₀ aryl group; and
R⁴ and R⁵ are, independently, H or a substituted or unsubstituted organic group comprising 1-20 carbon atoms; or, together with the atoms to which they are attached, R⁴ and R⁵ form a ring;
provided that Z is not tosyl.

In preferred compounds of the present invention, Z in formula (IX) is —CO₂R³ wherein R³ is a substituted or unsubstituted C₁₋₁₀ straight-chain or C₃₋₁₀ branched-chain alkyl group or a substituted or unsubstituted C₆₋₁₀ aryl group. Particularly preferred C₁₋₁₀-alkyl groups include methyl, ethyl, n-propyl, iso-propyl, n-butyl, sec-butyl, tert-butyl, pentyl, and hexyl, more preferably methyl, ethyl, iso-propyl, tert-butyl, even more preferably tert-butyl. Preferred C₆₋₁₀-aryl groups include phenyl, tolyl, xylyl, and methoxyphenyl, more preferably phenyl.

In preferred compounds of the present invention, Z in formula (IX) is —SO₂R″ wherein R″is a substituted or unsubstituted C₁₋₁₀ straight-chain or C₃₋₁₀ branched-chain alkyl group or a substituted or unsubstituted C₆₋₁₀ aryl group. Particularly preferred C₁₋₁₀-alkyl groups include methyl, trifluoromethyl, ethyl, n-propyl, iso-propyl, n-butyl, sec-butyl, tert-butyl, pentyl, and hexyl, more preferably methyl, ethyl, iso-propyl, and tert-butyl, even more preferably methyl and ethyl. Preferred C₆₋₁₀-aryl groups include phenyl, xylyl, methoxyphenyl, bromophenyl, and nitrophenyl.

In preferred compounds of the present invention, R⁴ and R⁵ in formula (IX) are, independently, H or a substituted or unsubstituted C₁₋₂₀ straight-chain or C₃₋₂₀ branched-chain alkyl group; or, together with the atoms to which they are attached, R⁴ and R⁵ form a ring.

In preferred compounds of the present invention, R⁴ and R⁵ in formula (IX) are, independently, H or a substituted or unsubstituted C₁₋₁₀ straight-chain or C₃₋₁₀ branched-chain alkyl group, more preferably H or a substituted or unsubstituted C₁₋₅ straight-chain or C₃₋₅ branched-chain alkyl group, even more preferably H.

In alternative preferred compounds of the present invention, together with the atoms to which they are attached, R⁴ and R⁵ in formula (IX) form a ring. More preferably, R⁴ and R⁵ form a ring which is —Bpin or —Bcat. Even more preferably, R⁴ and R⁵ form a ring which is —Bpin.

The present invention is directed to processes for preparing a compound of formula (VI), or salt or derivative thereof. The present invention is also directed to compounds of formulae (III), (V) and (IX), or salts or derivatives thereof. These compounds are intermediates in the synthesis of the compound of formula (VI). Preferred salts are those that retain the biological effectiveness and properties of the compounds and are formed from suitable non-toxic organic or inorganic acids. Acid addition salts are preferred. Representative examples of salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoroacetic acid and the like. The modification of a compound into a salt is a technique well known to chemists.

The present invention also provides a pharmaceutical composition comprising a compound as hereinbefore described. The pharmaceutical compositions of the present invention may take any conventional form.

The pharmaceutical compositions of the present invention may comprise one or more pharmaceutically acceptable carriers.

In the pharmaceutical compositions of the present invention, the compound of the present invention can be present alone or in combination with another active ingredient(s).

The present invention also provides compounds and compositions as hereinbefore described for use as a medicament.

The present invention also provides compounds and compositions as hereinbefore described for use in the treatment of cancer, preferably wherein the cancer is tenosynovial giant cell tumor (TGCT).

The present invention also provides the use of compounds and compositions as hereinbefore described for the manufacture of a medicament for the treatment of cancer, preferably wherein the cancer is tenosynovial giant cell tumor (TGCT).

The present invention also provides a process for the preparation of a compound of formula (VI), or a salt or derivative thereof,

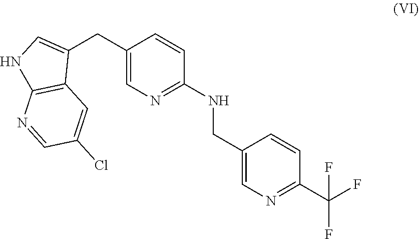 [SMILES: FC(F)(F)c1ccc(CNc2ccc(Cc3c[nH]c4ncc(Cl)cc34)cn2)cn1]

comprising the step of reducing a compound of formula (III)

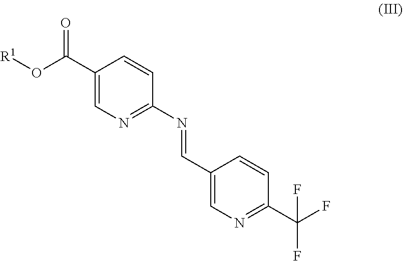

wherein R¹ is a substituted or unsubstituted C₁₋₂₀ straight-chain or C₃₋₂₀ branched-chain alkyl group or a substituted or unsubstituted C₆₋₂₀ aryl group;
with hydrogen in the presence of a Group 8 transition metal catalyst and a base to give a compound of formula (IV)

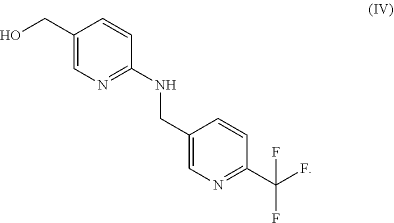 [SMILES: OCc1ccc(NCc2ccc(C(F)(F)F)nc2)nc1]

Preferred features of the step of reducing a compound of formula (III) are as described above.

Preferred steps to prepare the compound of formula (III) are as described above.

Preferred steps to transform the compound of formula (IV), or a salt thereof, into the compound of formula (VI) are as described above.

The present invention also provides a process for the preparation of a compound of formula (VI), or a salt or derivative thereof,

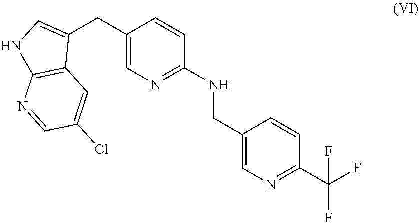 [SMILES: FC(F)(F)c1ccc(CNc2ccc(Cc3c[nH]c4ncc(Cl)cc34)cn2)cn1]

comprising the step of reacting a compound of formula (VIII)

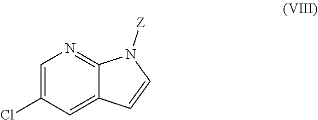

wherein Z is —SO₂R″ or —CO₂R³; and
R″ is a substituted or unsubstituted C₁₋₂₀ straight-chain or C₃₋₂₀ branched-chain alkyl group or a substituted or unsubstituted C₆₋₂₀ aryl group;
R³ is a substituted or unsubstituted C₁₋₂₀ straight-chain or C₃₋₂₀ branched-chain alkyl group or a substituted or unsubstituted C₆₋₂₀ aryl group;
with a borylation agent in the presence of a Group 9 transition metal catalyst to give a compound of formula (IX)

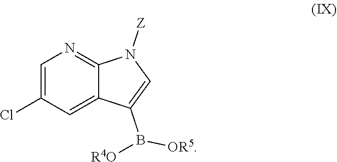

Preferred features of the step of reacting a compound of formula (VIII) with a borylation agent in the presence of a Group 9 transition metal catalyst are as described above.

Preferred steps to prepare the compound of formula (VIII) are as described above.

Preferred steps to transform the compound of formula (IX) into the compound of formula (VI) are as described above.

The invention will now be further described by way of the following non-limiting examples.

### EXAMPLES

### Materials

Ru-SNS, (dippf)PdCl₂ and chloro(1,5-cyclooctadiene)(1,10-phenanthroline)iridium(I)·THF adduct are commercially available from Johnson Matthey.

Bis(pinacolato)diboron, 5-chloro-1H-pyrrolo[2,3-b]pyridine, methyl 6-aminonicotinate, and 6-(trifluoromethyl)nicotinaldehyde are commercially available, e.g. from Combi-Blocks.

All solvents are commercially available, e.g. from Milipore-Sigma.

Di-tert-butyl dicarbonate, 4-dimethylaminopyridine, trifluoroacetic acid, potassium tert-butoxide, sodium methoxide and potassium carbonate are commercially available, e.g. from Milipore-Sigma.

Dimethyl carbonate is commercially available, e.g. from TCI America.

### Measurement Methods

Nuclear magnetic resonance (NMR) measurements were conducted using a Bruker Avance II 400 MHz.

### EXAMPLES

### Example 1: Preparation of methyl (E)-6-(((6-(trifluoromethyl)pyridin-3-yl)methylene)amino)nicotinate (Compound 3)

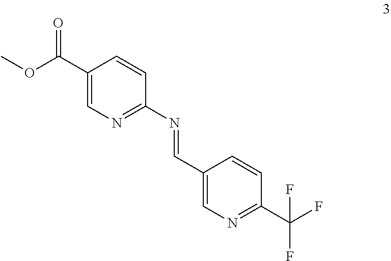 [SMILES: COC(=O)c1ccc(/N=C/c2ccc(C(F)(F)F)nc2)nc1]

To a 250 mL flask with stir bar open to air, methyl 6-aminonicotinate (Compound 1) (20 grams, 131 mmol, 1 equivalent) and 6-(trifluoromethyl)nicotinaldehyde (Compound 2) (24.2 grams, 138 mmol, 1.05 equivalence) were charged. To the flask, a Dean Stark trap attached to a condenser was attached. The flask was then sealed by a septa at the top of the condenser. The reactor was then degassed by three cycles of evacuation and filling by nitrogen. The reactor was then filled with 131 mL of toluene (1 M) by syringe. Agitation was started with a stir bar and trifluoroacetic acid (1.0 mL, 0.1 equivalence) was charged from a syringe. The reactor was then heated and allowed to reflux for 4 hours until the reaction was complete by disappearance of the amino pyridine 1. The reactor was cooled to 25° C. which led to the crystallization of the desired product. While the contents were agitated, the contents were canuled to a filter by vacuum. The reactor and was then washed with three 20 mL portions of toluene. The crystals were then dried under air using vacuum. This yielded 38.3 g (123.9 mmol, 90.1%) of a fluffy off-white solid (Compound 3).

¹H NMR (400 MHz, Chloroform-d) δ9.32 (s, 1H), 9.24 (d, J=2.0 Hz, 1H), 9.12 (d, J=2.3 Hz, 1H), 8.58-8.49 (m, 1H), 8.40 (dd, J=8.2, 2.3 Hz, 1H), 7.83 (d, J=8.1 Hz, 1H), 7.45 (d, J=8.2 Hz, 1H), 3.98 (s, 3H).

¹³C NMR (101 MHz, CDCl₃) δ189.50, 165.61, 162.87, 160.46, 151.87, 151.69, 151.00, 150.56, 139.86, 137.88, 137.52, 133.76, 125.27, 120.88, 120.85, 120.23, 52.64.

### Example 2: Preparation of (6-(((6-(trifluoromethyl)pyridin-3-yl)methyl)amino)pyridin-3-yl)methanol (Compound 4)

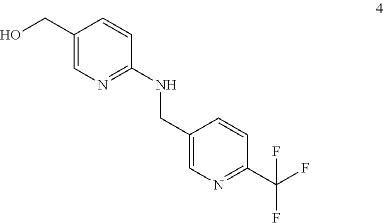 [SMILES: OCc1ccc(NCc2ccc(C(F)(F)F)nc2)nc1]

To a liner under an inert atmosphere, Compound 3 (500 mg, 1 equivalent) was charged, followed by potassium tert-butoxide (90 mg, 0.5 equivalence). The catalyst, RuSNS (20 mg, 2 mol %), was then charged to the liner. The liner was then loaded to the hydrogenator. The hydrogenator was then purged of oxygen by filling the hydrogenator with nitrogen followed by allowing pressure to be released. The purging procedure was repeated 5 times. To the hydrogenator ethanol (0.28 mL) in toluene (1.6 mL) was then delivered by syringe through an addition port. Agitation was then started (900 rpm), and the reactor was charged with hydrogen (28 bar). The reactor was then heated to 65° C. for 2 hours with a constant supply of hydrogen, before being raised to 120° C.for 8 hours. The reactor was then cooled and the pressure was relieved. Once the pressure was relieved, the liners were removed, and the crude mixture was diluted with a solution of MeOH/CH₂Cl₂. The diluted crude mixture was then passed over a pad of silica gel and the filtrate evaporated under reduced pressure. The crude oil was then diluted with CH₂Cl₂ and loaded onto a column of silica gel. The crude was then purified by column chromatography (2%-10% MeOH/CH₂Cl₂) to give 388 mg (1.37 mmol, 85%) of light yellow solid (Compound 4).

¹H NMR (400 MHz, Chloroform-d) δ8.69 (d, J=2.2 Hz, 1H), 8.03 (d, J=2.3 Hz, 1H), 7.84 (dd, J=8.1, 2.1 Hz, 1H), 7.62 (d, J=8.1 Hz, 1H), 7.47 (dd, J=8.5, 2.3 Hz, 1H), 6.40 (d, J=8.5 Hz, 1H), 5.04 (d, J=6.4 Hz, 1H), 4.66 (d, J=6.0 Hz, 2H), 4.53 (s, 2H), 1.98 (s, 1H).

¹³C NMR (101 MHz, CDCl₃) δ157.66, 149.36, 147.55, 138.77, 137.90, 136.40, 135.48, 126.35, 120.53, 120.48, 120.46, 120.44, 107.82, 62.90, 43.28.

¹⁹F NMR (376 MHz, CDCl₃) δ−67.74.

### Example 3: Preparation of Methyl ((6-(((6-(trifluoromethyl)pyridin-3-yl)methyl)amino)pyridin-3-yl)methyl) carbonate (Compound 5)

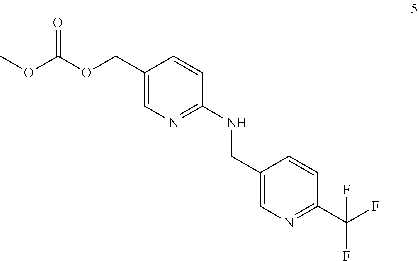 [SMILES: COC(=O)OCc1ccc(NCc2ccc(C(F)(F)F)nc2)nc1]

To a flask open to the air, Compound 4 (0.566 g, 2 mmol, 1 equivalent) was charged, followed by dimethyl carbonate (10.1 g, 9.43 mL). A Dean Stark trap and a condenser was attached to the flask. The reactor was sealed with a septa and nitrogen was allowed to flow to the reactor. The reactor was then warmed to 90° C. and a solution of sodium methoxide in methanol was charged to the flask by syringe (0.02 mL, 4.3 M, 0.05 equivalence). The reactor was then warmed to reflux. The distillate was then allowed to collect in the trap for 30 minutes. Once the Dean Stark trap was filled with distillate it was discarded and the trap was filled with dimethyl carbonate (15 mL). The solvent was allowed to reflux for 30 minutes, and then the solvent in the trap was removed. This process was repeated for 3 hours before full conversion was reached as identified by ¹H NMR. The reactor was then allowed to cool and the organic layer was washed with brine 3 times and the organic layer was dried over Na₂SO₄. The organic layer was then filtered and the solvent was evaporated. After cooling, crystals formed which were then collected to give Compound 5 (0.720 g, 1.8 mmol, 90%).

¹H NMR (400 MHz, Chloroform-d) δ8.68 (s, 1H), 8.10 (s, 1H), 7.83 (d, J=8.0 Hz, 1H), 7.60 (d, J=8.2 Hz, 1H), 7.46 (d, J=8.5 Hz, 1H), 6.38 (d, J=8.5 Hz, 1H), 5.32-5.18 (m, 1H), 5.00 (s, 2H), 4.65 (d, J=6.0 Hz, 2H), 3.75 (s, 3H).

¹³C NMR (101 MHz, CDCl₃) δ158.18, 155.83, 149.35, 149.31, 138.93, 138.69, 136.40, 120.79, 120.44, 120.41, 107.66, 67.58, 54.95, 43.11.

### Example 4: Preparation of tert-butyl 5-chloro-1H-pyrrolo[2,3-b]pyridine-1-carboxylate (Compound 8)

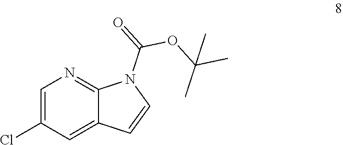 [SMILES: CC(C)(C)OC(=O)n1ccc2cc(Cl)cnc21]

To a flask open to the air, 5-chloro-1H-pyrrolo[2,3-b]pyridine (Compound 7) (21 g, 138 mmol, 1 equivalent) and 4-N,N-dimethylaniline (0.337 g, 0.0028 mmol, 0.02 equivalents) was charged. The flask was sealed with a septa and then purged of air by 3 cycles of evacuation and filling with nitrogen. To the reactor, THF was charged by syringe (120 mL). Agitation was started and then a solution of Boc₂O (36.1 g, 165.8 mmol, 1.1 equivalents) in THF (18 mL) was added to the reactor slowly, allowing for gas evolution to diminish. After addition of Boc₂O, the reactor was allowed to agitate for 1 hour and the crude material was analyzed by ¹H NMR. At this point, starting material 7 was no longer present. H₂O was then added to the reactor until no more gas evolved. Once complete, the H₂O was separated from the crude and the bulk of the solvent was evaporated by distillation. Once the majority of the solvent was distilled, toluene was added (100 mL). The solvent was then evaporated. This process of adding toluene and evaporating was repeated 3 times. Once the oil cooled, the product crystalized and was collected to give Compound 8 (33.4 g, 132 mmol, 96%).

¹H NMR (400 MHz, Chloroform-d) δ8.43 (d, J=2.3 Hz, 1H), 7.84 (d, J=2.3 Hz, 1H), 7.67 (d, J=4.0 Hz, 1H), 6.45 (d, J=4.1 Hz, 1H), 1.66 (s, 10H).

### Example 5: Preparation of tert-butyl 5-chloro-3-(4,4,5,5-tetramethyl-1,3,2-dioxaborolan-2-yl)-1H-pyrrolo[2,3-b]pyridine-1-carboxylate (Compound 9)

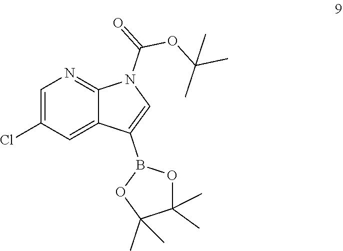 [SMILES: CC(C)(C)OC(=O)n1cc(B2OC(C)(C)C(C)(C)O2)c2cc(Cl)cnc21]

To a flask open to the air, chloro(1,5-cyclooctadiene)(1,10-phenanthroline)iridium(I). THF adduct (0.035 g, 0.000006 mmol, 0.001 equivalents) and 4,4,4′,4′,5,5,5′,5′-octamethyl-2,2′-bi(1,3,2-dioxaborolane, B₂pin₂) (10.58 g, 41.67 mmol, 0.7 equivalents) was charged to a flask. The flask was sealed and degassed by evacuation and refilling by nitrogen. The flask was then charged with heptanes (59 mL) and then iso-propanol (0.023 mL). Agitation was started and the flask was then warmed to 70° C. for 1 hour until it was a homogenous dark red colour. The flask was then opened while nitrogen was flowing into the flask and Compound 8 (15 g, 59.5 mmol, 1 equivalent) was poured into the catalyst solution. The reactor was sealed and allowed to a agitate while heating at 70° C. After 20 minutes, a white solid appeared and the solution changed from being homogenous to being a slurry. The reactor was allowed to heat for a further 40 minutes at 70° C. and then cooled. Once the reactor was cool, the slurry was cannulated to a filter. The reactor was washed with heptanes and the resultant mixture cannulated to the filter. The solid was then washed with heptanes and dried by passing air thereover to give Compound 9 (20.3 g, 53.5 mmol, 89%).

¹H NMR (400 MHz, Chloroform-d) δ8.43 (d, J=2.4 Hz, 1H), 8.22 (d, J=2.4 Hz, 1H), 8.07 (s, 1H), 1.66 (s, 10H), 1.37 (s, 13H).

¹³C NMR (101 MHz, CDCl₃) δ140.52, 136.79, 130.02, 123.51, 120.32, 120.19, 78.03, 77.00, 21.27, 18.06.

### Example 6: Preparation of methyl 5-((5-chloro-1H-pyrrolo[2,3-b]pyridin-3-yl)methyl)-N-((6-(trifluoromethyl)pyridin-3-yl)methyl)pyridin-2-amine (Compound 6)

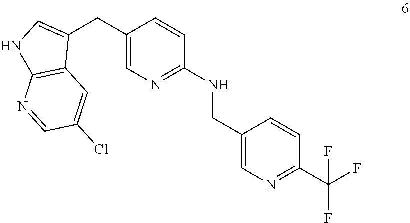 [SMILES: FC(F)(F)c1ccc(CNc2ccc(Cc3c[nH]c4ncc(Cl)cc34)cn2)cn1]

To a flask, (dippf)PdCl₂ (13.1 mg, 0.022 mmol, 2.5 mol %), Compound 5 (0.15 g, 0.38 mmol, 1 equivalent), K₂CO₃ (0.27 g, 0.76 mmol, 2 equivalents), and Compound 9 (0.365 g, 0.42 mmol, 1.1 equivalents) were measured. The flask was then sealed with septa and then evacuated and refilled with nitrogen. The purging process was repeated three times and then wet isopropanol (1.1 mL, 0.35 M), degassed by sparging with nitrogen for 2 hours, was added to the flask. Agitation was started and then the reactor was heated to 70° C. for 8 hours. After this time, the reaction mixture was cooled and extracted with ethyl acetate. The extract was then evaporated to dryness. The crude material was analyzed by ¹H NMR and found to have greater than 95% conversion to the desired product. Purification of the material by column chromatography (0-10% MeOH/CH₂Cl₂) gave Compound 6 (0.297 g, 0.712 mmol, 81%).

¹H NMR (400 MHz, Chloroform-d) δ9.63 (s, 1H), 8.72 (d, J=2.1 Hz, 1H), 8.21 (d, J=2.3 Hz, 1H), 8.10-7.99 (m, 1H), 7.91-7.81 (m, 1H), 7.73 (dd, J=2.3, 0.7 Hz, 1H), 7.63 (dd, J=8.1, 0.8 Hz, 1H), 7.28 (dd, J=8.4, 2.3 Hz, 1H), 7.09 (dd, J=2.4, 1.2 Hz, 1H), 6.35 (dd, J=8.5, 0.8 Hz, 1H), 5.04 (t, J=6.1 Hz, 1H), 4.65 (d, J=6.1 Hz, 2H), 3.91 (s, 2H).

¹⁹F NMR (376 MHz, CDCl₃) δ−67.74.

1-28. (canceled)

29. A process for the preparation of a compound of formula (VI), or a salt or derivative thereof, 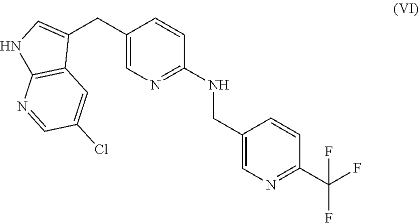 [SMILES: FC(F)(F)c1ccc(CNc2ccc(Cc3c[nH]c4ncc(Cl)cc34)cn2)cn1]
comprising reacting a compound of formula (V) 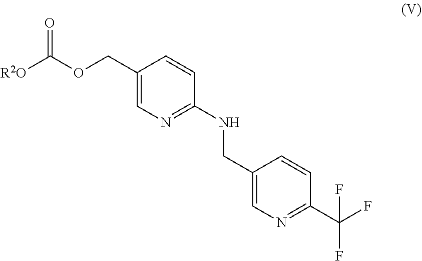
wherein R² is a substituted or unsubstituted C₁₋₂₀ straight-chain or C₃₋₂₀ branched-chain alkyl group, with a compound of formula (IX) 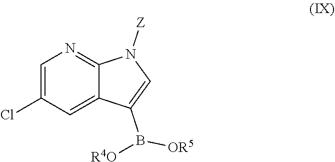
wherein
Z is —SO₂R″ or —CO₂R³;
R″ is a substituted or unsubstituted C₁₋₂₀ straight-chain or C₃₋₂₀ branched-chain alkyl group or a substituted or unsubstituted C₆₋₂₀ aryl group;
R³ is a substituted or unsubstituted C₁₋₂₀ straight-chain or C₃₋₂₀ branched-chain alkyl group or a substituted or unsubstituted C₆₋₂₀ aryl group; and
R⁴ and R⁵ are, independently, H or a substituted or unsubstituted organic group comprising 1-20 carbon atoms; or, together with the atoms to which they are attached, R⁴ and R⁵ form a ring;
in the presence of a Group 10 transition metal catalyst, a base, and water to give said compound of formula (VI).

30. A process as claimed in claim 1, wherein said Group 10 transition metal catalyst is a nickel catalyst, a palladium catalyst, or a platinum catalyst, preferably a palladium catalyst.

31. A process as claimed in claim 2, wherein said palladium catalyst is PdCl₂(dippf).

32. A process as claimed in claim 1, wherein the reacting of said compound of formula (V) with said compound of formula (IX) is carried out in a solvent which comprises an alcohol or a mixture of alcohols, preferably tert-amyl alcohol and/or isopropanol.

33. A process as claimed in claim 1, wherein said compound of formula (V) is prepared in situ in the presence of said compound of formula (IX), said Group 10 transition metal catalyst, said base and said water by reacting a compound of formula (IV), or a salt thereof, 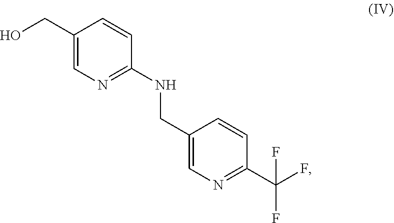 [SMILES: OCc1ccc(NCc2ccc(C(F)(F)F)nc2)nc1]
with a dialkyl carbonate of formula R²O(CO)OR², wherein each R² is a substituted or unsubstituted C₁₋₂₀ straight-chain or C₃₋₂₀ branched-chain alkyl group.

34. A process as claimed in claim 1, wherein said compound of formula (V) is prepared in situ in the presence of said compound of formula (IX), said Group 10 transition metal catalyst, said base and said water by reacting a compound of formula (IV), or a salt thereof, 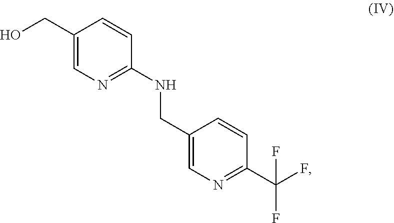 [SMILES: OCc1ccc(NCc2ccc(C(F)(F)F)nc2)nc1]
with a dialkyl carbonate of formula R²O(CO)OR², wherein each R² is a substituted or unsubstituted C₁₋₂₀ straight-chain or C₃₋₂₀ branched-chain alkyl group, and
wherein said compound of formula (IV) is prepared by reducing a compound of formula (III) 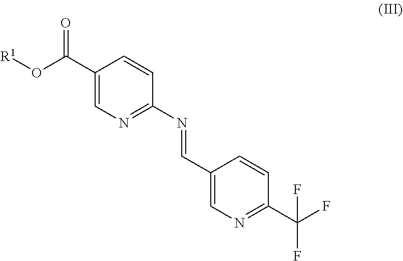
wherein R¹ is a substituted or unsubstituted C₁₋₂₀ straight-chain or C₃₋₂₀ branched-chain alkyl group or a substituted or unsubstituted C₆₋₂₀ aryl group;
with hydrogen in the presence of a Group 8 transition metal catalyst and a base.

35. A process as claimed in claim 6, wherein said Group 8 transition metal catalyst is an iron catalyst, a ruthenium catalyst, or an osmium catalyst, preferably a ruthenium catalyst, more preferably Ru-SNS.

36. A process as claimed in claim 6, wherein said compound of formula (III) is prepared by reacting a compound of formula (I) 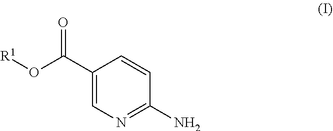
wherein R¹ is as hereinbefore defined,
with a compound of formula (II) 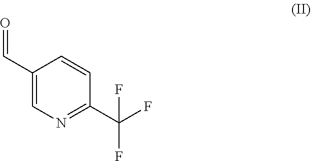 [SMILES: O=Cc1ccc(C(F)(F)F)nc1]
in the presence of an acid catalyst.

37. A process as claimed in claim 1, wherein said compound of formula (V) is prepared by reacting a compound of formula (IV), or a salt thereof, 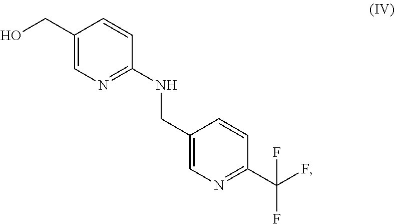 [SMILES: OCc1ccc(NCc2ccc(C(F)(F)F)nc2)nc1]
with a dialkyl carbonate of formula R²O(CO)OR², wherein each R² is a substituted or unsubstituted C₁₋₂₀ straight-chain or C₃₋₂₀ branched-chain alkyl group, in the presence of a base.

38. A process as claimed in claim 1, wherein said compound of formula (V) is prepared by reacting a compound of formula (IV), or a salt thereof, 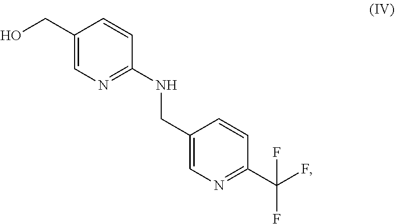 [SMILES: OCc1ccc(NCc2ccc(C(F)(F)F)nc2)nc1]
with a dialkyl carbonate of formula R²O(CO)OR², wherein each R² is a substituted or unsubstituted C₁₋₂₀ straight-chain or C₃₋₂₀ branched-chain alkyl group, in the presence of a base, and
wherein said compound of formula (IV) is prepared by reducing a compound of formula (III) 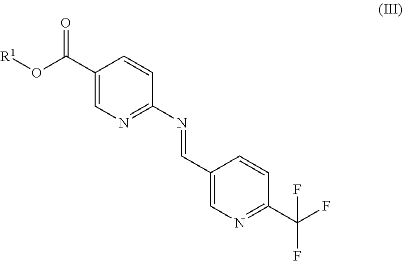
wherein R¹ is a substituted or unsubstituted C₁₋₂₀ straight-chain or C₃₋₂₀ branched-chain alkyl group or a substituted or unsubstituted C₆₋₂₀ aryl group;
with hydrogen in the presence of a Group 8 transition metal catalyst and a base.

39. A process as claimed in claim 10, wherein said Group 8 transition metal catalyst is an iron catalyst, a ruthenium catalyst, or an osmium catalyst, preferably a ruthenium catalyst, more preferably Ru-SNS.

40. A process as claimed in claim 10, wherein said compound of formula (III) is prepared by reacting a compound of formula (I) 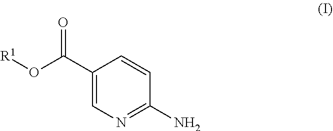
wherein R¹ is as hereinbefore defined,
with a compound of formula (II) 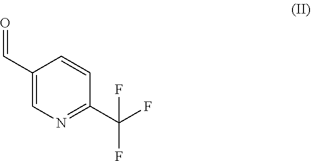 [SMILES: O=Cc1ccc(C(F)(F)F)nc1]
in the presence of an acid catalyst.

41. A process as claimed in claim 1, wherein said compound of formula (IX) is prepared by reacting a compound of formula (VIII) 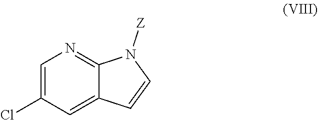
wherein Z is —SO₂R″ or —CO₂R³;
R″ is a substituted or unsubstituted C₁₋₂₀ straight-chain or C₃₋₂₀ branched-chain alkyl group or a substituted or unsubstituted C₆₋₂₀ aryl group; and
R³ is a substituted or unsubstituted C₁₋₂₀ straight-chain or C₃₋₂₀ branched-chain alkyl group or a substituted or unsubstituted C₆₋₂₀ aryl group;
with a borylation agent in the presence of a Group 9 transition metal catalyst.

42. A process as claimed in claim 13, wherein said compound of formula (VIII) is prepared by reacting a compound of formula (VII) 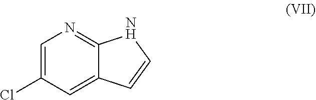
with a sulfonyl halide of formula R″SO₂X^{a} wherein X^{a} is a halide and R″ is as hereinbefore defined, or a compound of formula (R³O(CO))₂O, wherein R³ is as hereinbefore defined.

43. A process for the preparation of a compound of formula (VI), or a salt or derivative thereof, 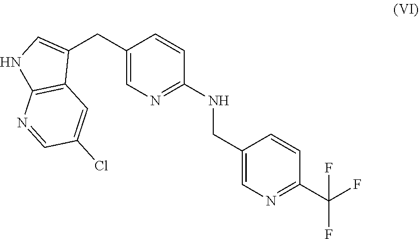 [SMILES: FC(F)(F)c1ccc(CNc2ccc(Cc3c[nH]c4ncc(Cl)cc34)cn2)cn1]
comprising:
reacting a compound of formula (I) 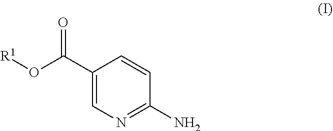
wherein R¹ is a substituted or unsubstituted C₁₋₂₀ straight-chain or C₃₋₂₀ branched-chain alkyl group or a substituted or unsubstituted C₆₋₂₀ aryl group,
with a compound of formula (II), 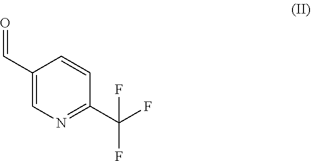 [SMILES: O=Cc1ccc(C(F)(F)F)nc1]
in the presence of an acid catalyst, to give a compound of formula (III), 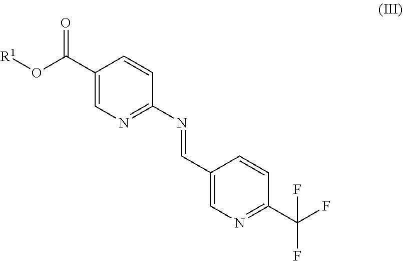
wherein R¹ is as hereinbefore defined;
reducing said compound of formula (III) with hydrogen in the presence of a Group 8 transition metal catalyst and a first base to give a compound of formula (IV) 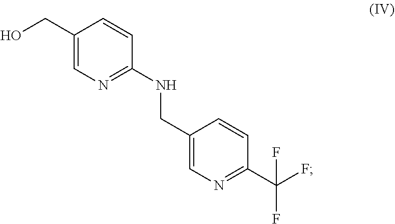 [SMILES: OCc1ccc(NCc2ccc(C(F)(F)F)nc2)nc1]
reacting said compound of formula (IV), or a salt thereof, with a dialkyl carbonate of formula R²O(CO)OR², wherein each R² is a substituted or unsubstituted C₁₋₂₀ straight-chain or C₃₋₂₀ branched-chain alkyl group, in the presence of a second base to give a compound of formula (V), 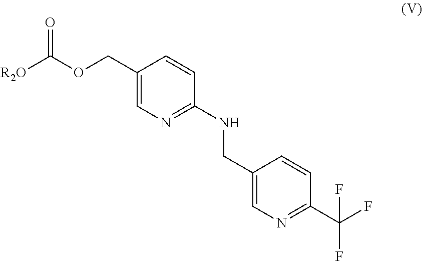
wherein R² is as hereinbefore defined;
reacting a compound of formula (VII) 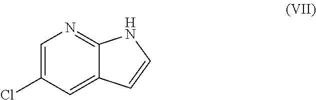
with a sulfonyl halide of formula R″SO₂X^{a} wherein X^{a} is a halide and R″ is a substituted or unsubstituted C₁₋₂₀ straight-chain or C₃₋₂₀ branched-chain alkyl group or a substituted or unsubstituted C₆₋₂₀ aryl group, or a compound of formula (R³O(CO))₂O, wherein R³ is a substituted or unsubstituted C₁₋₂₀ straight-chain or C₃₋₂₀ branched-chain alkyl group or a substituted or unsubstituted C₆₋₂₀ aryl group, to give a compound of formula (VIII) 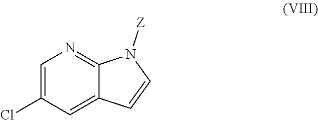
wherein Z is —SO₂R″ or —CO₂R³, and
R″ and R³ are as hereinbefore defined;
reacting said compound of formula (VIII) with a borylation agent in the presence of a Group 9 transition metal catalyst to give a compound of formula (IX) 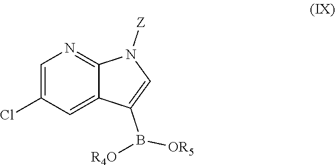
wherein Z is as hereinbefore defined,
R⁴ and R⁵ are, independently, H or a substituted or unsubstituted organic group comprising 1-20 carbon atoms; or, together with the atoms to which they are attached, R⁴ and R⁵ form a ring; and reacting said compound of formula (V) with said compound of formula (IX) in the presence of a Group 10 transition metal catalyst, a third base, and water to give said compound of formula (VI).

44. A process for the preparation of a compound of formula (VI), or a salt or derivative thereof, 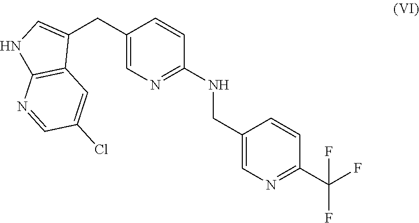 [SMILES: FC(F)(F)c1ccc(CNc2ccc(Cc3c[nH]c4ncc(Cl)cc34)cn2)cn1]
comprising:
reacting a compound of formula (I) 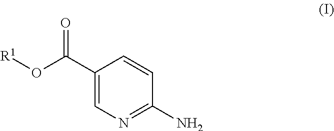
wherein R¹ is a substituted or unsubstituted C₁₋₂₀ straight-chain or C₃₋₂₀ branched-chain alkyl group or a substituted or unsubstituted C₆₋₂₀ aryl group,
with a compound of formula (II), 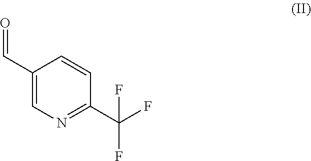 [SMILES: O=Cc1ccc(C(F)(F)F)nc1]
in the presence of an acid catalyst, to give a compound of formula (III), 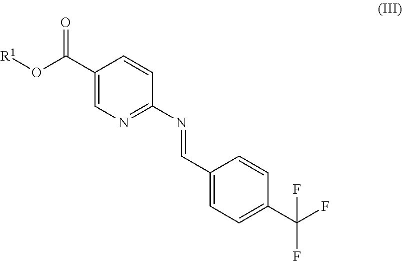
wherein R¹ is as hereinbefore defined;
reducing said compound of formula (III) with hydrogen in the presence of a Group 8 transition metal catalyst and a first base to give a compound of formula (IV) 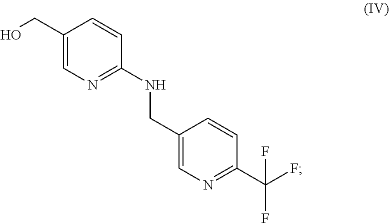 [SMILES: OCc1ccc(NCc2ccc(C(F)(F)F)nc2)nc1]
reacting a compound of formula (VII) 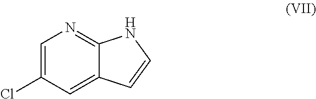
with a sulfonyl halide of formula R″SO₂X^{a} wherein X^{a} is a halide and R″ is a substituted or unsubstituted C₁₋₂₀ straight-chain or C₃₋₂₀ branched-chain alkyl group or a substituted or unsubstituted C₆₋₂₀ aryl group, or a compound of formula (R³O(CO))₂O, wherein R³ is a substituted or unsubstituted C₁₋₂₀ straight-chain or C₃₋₂₀ branched-chain alkyl group or a substituted or unsubstituted C₆₋₂₀ aryl group, to give a compound of formula (VIII) 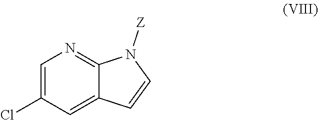
wherein Z is —SO₂R″ or —CO₂R³, and
R″ and R³ are as hereinbefore defined;
reacting said compound of formula (VIII) with a borylation agent in the presence of a Group 9 transition metal catalyst to give a compound of formula (IX) 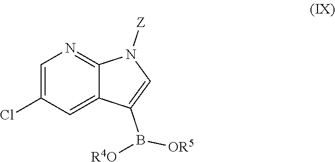
wherein Z is as hereinbefore defined,
R⁴ and R⁵ are, independently, H or a substituted or unsubstituted organic group comprising 1-20 carbon atoms; or, together with the atoms to which they are attached, R⁴ and R⁵ form a ring; and
reacting a compound of formula (V) 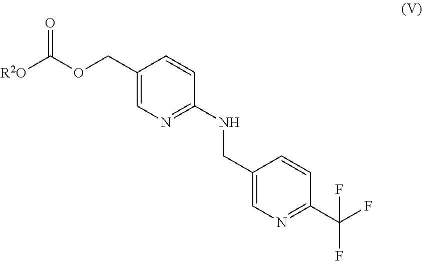
wherein R² is a substituted or unsubstituted C₁₋₂₀ straight-chain or C₃₋₂₀ branched-chain alkyl group, with said compound of formula (IX) in the presence of a Group 10 transition metal catalyst, a third base, and water to give said compound of formula (VI),
wherein said compound of formula (V) is prepared in situ in the presence of the compound of formula (IX), the Group 10 transition metal catalyst, the third base and water by reacting said compound of formula (IV), or a salt thereof, with a dialkyl carbonate of formula R²O(CO)OR²,
wherein each R² is a substituted or unsubstituted C₁₋₂₀ straight-chain or C₃₋₂₀ branched-chain alkyl group.

45. A process as claimed in claim 1, wherein the compound of formula (V) is

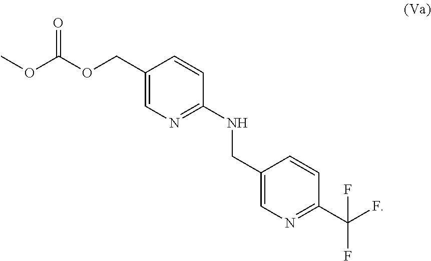 [SMILES: COC(=O)OCc1ccc(NCc2ccc(C(F)(F)F)nc2)nc1]

46. A process as claimed in claim 1, wherein the compound of formula (IX) is

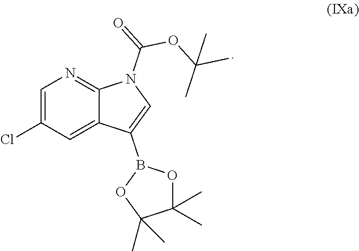 [SMILES: CC(C)(C)OC(=O)n1cc(B2OC(C)(C)C(C)(C)O2)c2cc(Cl)cnc21]

47. A process as claimed in claim 1, wherein the reaction of the compound of formula (V) with the compound of formula (IX) takes place in the presence of a Lewis acid.
